(19) United States
(12) Patent Application Publication
(10) Pub. No.: US 2009/0172828 A1
(43) Pub. Date: Jul. 2, 2009

(54) BYDV MP IS A VIRAL DETERMINANT RESPONSIBLE FOR PLANT GROWTH RETARDATION

(75) Inventors: Richard Y. Zhao, Brookeville, MD (US); Daowen Wang, Beijing (CN)

Correspondence Address: FULBRIGHT & JAWORSKI, LLP, 1301 MCKINNEY, SUITE 5100, HOUSTON, TX, 77010-3095, US

(73) Assignee: The University of Maryland, Baltimore

(21) Appl. No.: 12/097,215

(22) PCT Filed: Dec. 13, 2006
(86) PCT No.: PCT/US2006/047472

§ 371 Date: Oct. 22, 2008

Related U.S. Application Data
(60) Provisional application No. 60/750,028, filed on Dec. 13, 2005.

(51) Int. Cl. G01N 33/53; A01H 5/00; C12Q 1/68; C12Q 1/02

(52) U.S. Cl. 800/3; 800/301; 435/6; 435/29

(57) ABSTRACT

The present invention concerns the barley yellow dwarf virus (BYDV) and compositions and methods related thereto. In particular in the invention, identifies a movement protein (MP) of BYDV as being responsible for at least one symptom produced as a result of BYDV infection. In certain aspects, the invention concerns a target to inhibit BYDV and to inhibit at least one symptom of BYDV infection, for example, at least plant growth retardation. In particular aspects, the invention relates to screening methods for identifying suppressors of BYDV MP.

### CROSS-REFERENCE TO RELATED APPLICATIONS

This application claims priority to U.S. Provisional Patent Application Ser. No. 60/750,028, filed Dec. 13, 2005, which is incorporated by reference herein in its entirety.

### TECHNICAL FIELD

In certain embodiments of the invention, the field of the invention includes plant biology, yeast biology, agriculture, molecular biology, and/or cell biology, for example.

### BACKGROUND OF THE INVENTION

Barley yellow dwarf virus (BYDV) is a global viral disease of cereals that is one of the most destructive of viral diseases of cereals (Miller and Rasochova, 1997). Stunted growth of plants is one of the primary symptoms of BYDV infections, which results in significant yield loss and thus has enormous economical impact on crop production. The virus is transmitted by aphids and infects a wide host range including wheat, oats and barley. The primary symptoms of BYDV infections are stunting and discoloration of the leaf tips such as yellowing, reddening or purpling. BYDV has been found world-wide in Asia, Australia, Africa, Canada, Europe, New Zealand, South America and in the U.S. BYDV infections are unpredictable and there are currently very few options for controlling it. The options that exist are generally ineffective, impractical, or too expensive (Ministry of Agriculture and Food, Canada, 2003).

### SUMMARY OF THE INVENTION

The present invention concerns identification of barley yellow dwarf virus (BYDV) movement protein (MP) as being related to pathogenesis of a plant infected therewith. Therefore, in certain aspects of the invention, BYDV MP is identified as being useful to target and/or inhibit one or more symptoms of BYDV infection. In particular aspects, there is a system that can be utilized to screen for one or more compounds useful to target BYDV MP, for example, to inhibit its expression, production, and/or activity. Although the screening system may be of any suitable kind so long as it is able to identify one or more compounds that inhibit MP expression, production, and/or activity, in particular embodiments the system of the present invention is an in vitro system or an in vivo system. In additional embodiments, the system utilizes eukaryotic cells, including plant or animal cells, for example, and in further embodiments, the system utilizes prokaryotic cells. Exemplary prokaryotic organisms that may be used include, for example, E. coli, Listeria monocytogenes, Lactic acid bacteria, B. subtilis, and Pseudomonas. Exemplary embodiments of eukaryotic organisms or cells thereof include, at least, yeast, human, mouse, rat, Xenopus, C. elegans, Arabidopsis, zebrafish, Neurospora, Drosophila melanogaster, Spisula solidissima, Candida, Pichia, Saccharomyces spp., Schziosaccharomyces spp. and filamentous fungi such as Asperigillus and Penicillium. Exemplary disclosure herein describes yeast and transgenic plants, but one of skill in the art recognizes that the present invention encompasses any transgenic organism, including mouse, rat, Xenopus, C. elegans, Arabidopsis, zebrafish, Neurospora, Drosophila melanogaster, and Spisula solidissima, for example.

In certain screening methods of the invention, a BYDV MP is engineered into a cell or at least one cell of an organism, such as by genetically engineering a DNA encoding part or all of the BYDV MP into the genome of the cell. Such an engineered cell or organism has at least one detectable phenotype as a direct or indirect result of the presence of BYDV MP in the cell. The cell is subjected to one or more test agents, and the detectable phenotype is observed, assayed, or monitored. When the detectable phenotype is affected upon delivery of the test agent, the test agent may be further defined as a suppressor (which may be referred to as an inhibitor) of BYDV MP. The suppressor may inhibit activity of the BYDV MP protein, inhibit the expression of the BYDV MP gene into BYDV MP RNA, and/or inhibit translation of the BYDV MP RNA into protein. Exemplary phenotypes include cell proliferation, cell growth, organismal growth, cell viability, cell culture viability, cell shape, chromosomal abnormality; cellular toxicity; plant growth, and so forth. Exemplary test agents include nucleic acids, proteins, polypeptides, peptides, small molecules, mixtures thereof, combinations thereof, and so forth. In specific aspects, the suppressor inhibits the portion of MP that assists in the transport of viral genomic RNA across the nuclear envelope, for example. In other aspects, the suppressor inhibits at least part of a N-terminal amphiphilic α-helix of MP, protein dimerization; single-strand RNA binding; presence of a novel nuclear envelope (NE)-targeting domain, and an argenine-rich RNA binding motif (Xia et al., 2006).

In one embodiment of the invention, there is a method of identifying a suppressor of barley yellow dwarf virus movement protein (BYDV MP), comprising: (a) providing a candidate suppressor; (b) admixing the candidate suppressor with an isolated compound or cell, or a suitable experimental animal to produce a recombinant compound or cell, or a suitable experimental animal; (c) measuring one or more characteristics of the recombinant compound, cell or animal in step (b); and (d) comparing the characteristic measured in step (c) with the characteristic of the compound, cell or animal in the absence of said candidate suppressor, wherein a difference between the measured characteristics indicates that said candidate suppressor is a suppressor of the compound, cell or animal.

In one embodiment of the invention, there is a method of screening for a suppressor that downregulates barley yellow dwarf virus (BYDV) movement protein (MP) activity, comprising: (a) contacting a cell culture with a test agent, wherein the cell culture comprises a plurality of cells having an integrated BYDV MP gene or a variant thereof under control of an inducible promoter; (b) growing the culture under conditions suitable to induce expression of the BYDV MP gene or variant thereof; (c) screening the test culture for a cell characteristic or phenotype not present in a control cell culture, wherein the presence of the cell characteristic or cell phenotype indicates that the test agent is a suppressor that down-regulates BYDV MP activity. In a specific embodiment, the BYDV MP gene comprises DNA SEQ ID NO:2. In another specific embodiment, the cell characteristic is increased viability compared to the control culture. In a further specific embodiment, the cell phenotype is normal cell length and size. In an additional specific embodiment, the test agent prevents BYDV MP-induced cell cycle G2 arrest. In another embodiment, the yeast is a fission yeast, such as Schizosaccharomyces pombe, for example. In an alternative embodiment, the yeast is a budding yeast, such as Saccharomyces cerevisiae, for example.

In an additional embodiment of the invention, there is a BYDV MP suppressor identified using any method of the invention. In a further embodiment, there is a viricide composition comprising a BYDV MP suppressor of the invention.

In another embodiment of the invention, there is an isolated yeast cell transformed with a vector having DNA encoding barley yellow dwarf virus (BYDV) movement protein (MP) or a variant thereof and the regulatory elements necessary to express the DNA in the yeast cell. In specific embodiments, the yeast is a fission yeast, such as Schizosaccharomyces pombe, for example, or budding yeast, such as Saccharomyces cerevisiae, for example.

In a further embodiment of the invention, there is an in vitro screening method to identify an agent that binds to BYDV MP or a variant thereof, the method comprising (a) contacting BYDV MP or variant thereof with a test agent under conditions that allow a complex to form between the BYDV MP or variant and the test agent, (b) detecting complex formation, wherein the presence of the complex identifies the test agent as an agent that binds to BYDV MP or to the variant. In a specific embodiment, BYDV MP comprises the amino acid sequence of SEQ ID NO. 1. In an additional specific embodiment, BYDV MP is encoded by a nucleotide sequence of SEQ ID NO. 2. In particular aspects of the invention, screening occurs in a multi-well plate as part of a high-throughput screen, for example.

In one embodiment of the invention, there is a method for screening to identify a suppressor that downregulates barley yellow dwarf virus (BYDV) movement protein (MP) activity, comprising: (a) contacting the BYDV MP or a variant thereof with a test agent under conditions suitable to allow a complex to form between BYDV MP and the test agent, (b) detecting whether or not a complex is formed between the BYDV MP or variant and the test agent, wherein the presence of the complex identifies the test agent as a candidate suppressor of BYDV MP or the variant activity, (c) if a complex is formed, selecting the candidate suppressor of step (b), (d) establishing a test cell culture and a control cell culture wherein both cultures comprise a plurality of cells having an integrated BYDV MP gene or variant thereof under operable control of an inducible promoter, (e) contacting the test cell culture with the candidate suppressor of step (c), (f) growing the test and control cultures under conditions suitable to induce expression of the BYDV MP gene or the variant thereof; (g) screening the test culture for a cell characteristic or phenotype not present in a control culture, wherein the presence of the cell characteristic or cell phenotype indicates that the candidate suppressor is a suppressor that downregulates BYDV MP activity or activity of the variant. In a specific embodiment, the cell is a yeast cell or a plant cell. In specific embodiments, the BYDV MP gene has DNA SEQ. ID NO. 2 and the cell characteristic is increased viability of the cells in the test culture compared to the viability of the cells in the control culture. In another specific embodiment, the BYDV MP gene has DNA SEQ. ID NO. 2 and the cell phenotype is normal length or size of the cells in the test culture compared to elongated or larger cells in the control culture. In a further specific embodiment, the BYDV MP gene has DNA SEQ. ID NO. 2 and the characteristic is a normal G2 cell cycle. In a specific embodiment, the plant is A. thaliana.

In an additional embodiment of the invention, there is a method for screening to identify an agent that alters the expression of barley yellow dwarf virus (BYDV) movement protein (MP) activity in a cell transformed to express BYDV or a variant thereof, comprising: (a) determining separately a control group and a test group of transformed cells, each group of transformed cells genetically engineered to express (i) the BYDV MP or variant thereof under control of a promoter, and (ii) a reporter gene whose expression is increased in response to expression of the BYDV viral movement protein or variant thereof; (b) contacting a test agent with the test group, (c) incubating both groups under identical conditions that permit the cells to grow and divide, (d) measuring and comparing the level of expression of the reporter in both the test and control groups, wherein a difference indicates that the test agent alters the expression of the BYDV viral movement protein or variant thereof. In a specific embodiment of the invention, the BYDV MP further comprises a selectable marker.

In another embodiment of the invention, there is a transgenic plant resistant to barley yellow dwarf virus (BYDV) infection, which plant comprises a plurality of plant cells transformed with a vector to express inhibitory RNA (such as, for example, small inhibitory RNA (microRNA and RNAi)) that downregulates expression, transcription or translation of BYDV MP. In a specific embodiment, the inhibitory RNA is anti-sense RNA. In a further specific embodiment, the inhibitory RNA is cosuppressor RNA. In additional embodiments of the invention, the BYDV movement protein is encoded by a DNA molecule having SEQ. ID NO. 2. In another specific embodiment, the plant is selected from the group consisting of A. thaliana and tobacco. In one aspect of the invention, the plant is a member of the group of host plants naturally infected by BYDV comprising barley, wheat, oats and corn. In a specific embodiment, the vector is a viral vector obtained from a positive single-stranded RNA plant virus. In an additional specific embodiment, the positive single-stranded RNA plant virus is a tobamovirus. In certain aspects of the invention, the tobamovirus is a tobacco mosaic virus.

In one embodiment of the invention, there is a method of screening for a suppressor that downregulates barley yellow dwarf virus (BYDV) movement protein (MP) activity in yeast cells, comprising: (a) contacting a yeast cell culture with a test agent, wherein the yeast cell culture comprises a plurality of yeast cells having an integrated BYDV MP gene or a variant thereof under control of inducible promoter; (b) growing the culture under conditions suitable to induce expression of the BYDV MP gene or variant thereof; (c) screening the test culture for a cell characteristic or phenotype not present in a control yeast cell culture, wherein the presence of the cell characteristic or cell phenotype indicates that the test agent is a suppressor that downregulates BYDV MP activity. In a specific embodiment, the cell characteristic is increased viability compared to the control culture. In an additional specific embodiment, the cell phenotype is normal cell length and size. In one aspect of the invention, the test agent prevents BYDV MP-induced cell cycle G2 arrest.

In other embodiments of the invention, there is a BYDV MP suppressor identified using a method of the invention. In additional embodiments, there is a viricide composition comprising a BYDV MP suppressor.

In further embodiments of the invention, there is an isolated yeast cell transformed with a vector having DNA encoding barley yellow dwarf virus (BYDV) movement protein (MP) or a variant thereof and the regulatory elements necessary to express the DNA in the yeast cell.

In additional embodiments of the invention, there is an in vitro screening method to identify an agent that binds to BYDV MP or a variant thereof, the method comprising (a) contacting BYDV MP or variant thereof with a test agent under conditions that allow a complex to form between the BYDV MP or variant and the test agent, (b) detecting complex formation, wherein the presence of the complex identifies the test agent as an agent that binds to BYDV MP or to the variant. In specific aspects, the screening occurs in a multi-well plate.

In yet another embodiment of the invention, there is a method for screening to identify a suppressor that downregulates barley yellow dwarf virus (BYDV) movement protein (MP) activity, comprising: (a) contacting the BYDV MP or a variant thereof with a test agent under conditions suitable to allow a complex to form between BYDV MP and the test agent, (b) detecting whether or not a complex is formed between the BYDV MP or variant and the test agent, wherein the presence of the complex identifies the test agent as a candidate suppressor of BYDV MP or the variant activity, (c) if a complex is formed, selecting the candidate suppressor of step (b), (d) establishing a test cell culture and a control cell culture wherein both cultures comprise a plurality of cells having an integrated BYDV MP gene or variant thereof under operable control of an inducible promoter, (e) contacting the test cell culture with the candidate suppressor of step (c), (f) growing the test and control cultures under conditions suitable to induce expression of the BYDV MP gene or the variant thereof; and (g) screening the test culture for a cell characteristic or phenotype not present in a control culture, wherein the presence of the cell characteristic or cell phenotype indicates that the candidate suppressor is a suppressor that downregulates BYDV MP activity or activity of the variant. Any cell for the invention may be a yeast cell or a plant cell. In a specific embodiment, the BYDV MP gene comprises DNA of SEQ ID NO:2 and the cell characteristic comprises increased viability of the cells in the test culture compared to the viability of the cells in the control culture. In another embodiment of the invention, the BYDV MP gene comprises DNA of SEQ ID NO:2 and the cell phenotype comprises normal length and/or size of the cells in the test culture compared to elongated and/or larger cells in the control culture. In further specific embodiments, the BYDV MP gene comprises DNA of SEQ ID NO:2 and the characteristic comprises a normal G2 cell cycle.

In another embodiment of the invention, there is a method for screening to identify an agent that alters the expression of barley yellow dwarf virus (BYDV) movement protein (MP) activity in a cell transformed to express BYDV or a variant thereof, comprising: (a) determining separately a control group and a test group of transformed cells, each group of transformed cells genetically engineered to express (i) the BYDV MP or variant thereof under control of a promoter, and (ii) a reporter gene whose expression is increased in response to expression of the BYDV viral movement protein or variant thereof; (b) contacting a test agent with the test group; (c) incubating both groups under identical conditions that permit the cells to grow and divide; and (d) measuring and comparing the level of expression of the reporter in both the test and control groups, wherein a difference indicates that the test agent alters the expression of the BYDV viral movement protein or variant thereof. In a specific aspect, the BYDV MP further comprises a selectable marker.

In an additional embodiment, there is a transgenic plant resistant to barley yellow dwarf virus (BYDV) infection, wherein the plant comprises a plurality of plant cells transformed with a vector to express inhibitory RNA that downregulates expression, transcription or translation of BYDV MP. In a specific embodiment, the inhibitory RNA comprises anti-sense RNA, cosuppressor RNA, or a mixture thereof.

In certain transgenic plants of the invention, the BYDV movement protein is encoded by a DNA molecule having SEQ ID NO:2. In a specific embodiment, the plant is selected from the group consisting of A. thaliana and tobacco. In a further specific embodiment, the plant is a member of the group of host plants naturally infected by BYDV, and in additional specific embodiments, said group comprise barley, wheat, oats and corn. In one aspect of the invention, the vector comprises a viral vector obtained from a positive single-stranded RNA plant virus, such as a tobamovirus, for example, such as tobacco mosaic virus.

In one embodiment of the invention, there is a transgenic plant stably transformed with a construct of the invention, such as one that comprises a suppressor of BYDV MP. In a specific embodiment, the construct further comprises a selected coding region operably linked to the region that encodes the suppressor. In additional embodiments, the construct or an additional construct in the plant further comprises a region that encodes an insect resistance protein, a bacterial disease resistance protein, a fungal disease resistance protein, a viral disease resistance protein, a nematode disease resistance protein, a herbicide resistance protein, a protein affecting grain composition or quality, a nutrient utilization protein, an environment or stress resistance protein, a mycotoxin reduction protein, a male sterility protein, a selectable marker protein, a screenable marker protein, a negative selectable marker protein, or a protein affecting plant agronomic characteristics. In a specific embodiment, a selectable marker protein is selected from the group consisting of phosphinothricin acetyltransferase, glyphosate resistant EPSPS, aminoglycoside phosphotransferase, hygromycin phosphotransferase, neomycin phosphotransferase, dalapon dehalogenase, bromoxynil resistant nitrilase, anthranilate synthase and glyphosate oxidoreductase. The selected coding region may be operably linked to a terminator.

In other embodiments, there is one or more transgenic plants that express BYDV MP, for example to utilize as an in vivo system to identify suppressors of BYDV MP. For example, a transgenic plant that produces BYDV MP in one or more cells is subjected to one of more test compounds, such as via a water source, topically, by gun, engineering another suppressor gene into the plant to make it MP or viral resistant; genetic selection of new crop variants to specifically target suppression of MP, and so forth. Other transgenic plants of the invention may be exposed to test compounds in this manner. In further embodiments of transgenic plants of the invention, one or more cells of the plant expresses a gene product (such as RNA or protein) known to inhibit BYDV MP production, function, or expression. In

In additional embodiments of the invention, including DNA constructs or stably transformed plants comprising one or more DNA constructs, there is a terminator and/or an enhancer. The construct may comprise plasmid DNA. The construct may comprise a transit peptide coding sequence, such as, for example, chlorophyll a/b binding protein transit peptide, small subunit of ribulose bisphosphate carboxylase transit peptide, EPSPS transit peptide or dihydrodipocolinic acid synthase transit peptide.

Transgenic plants of the invention include monocotyledonous plants, such as a monocotyledonous plant selected from the group consisting of corn, wheat, maize, rye, rice, oat, barley, turfgrass, sorghum, millet, sugarcane, pineapples, dates, bananas, bamboo, and palms, for example. The transgenic plant may be further defined as a dicotyledonous plant, such as one selected from the group consisting of Arabidopsis, tobacco, tomato, potato, soybean, cotton, canola, alfalfa, sunflower, carrot, parsley, coriander, fennel, rose and dill, for example. The transgenic plant may be further defined as a fertile R0 transgenic plant.

In additional embodiments of the invention, there is a seed of the fertile R0 transgenic plant of the invention, wherein said seed comprises said selected DNA. The plant may be further defined as a progeny plant of any generation of a fertile R0 transgenic plant, wherein said fertile R0 transgenic plant comprises said selected DNA. In other embodiments, there are seed of the progeny plant, wherein said seed comprises said selected DNA.

In an additional embodiment, there is a transgenic plant cell stably transformed with a selected DNA comprising a suppressor of BYDV MP. The cell may be further defined as located within a seed. The cell may be further defined as located within a plant. In additional aspects, there is a tissue culture comprising the transgenic plant cell.

In additional embodiments of the invention, there is a method of expressing a selected protein in a transgenic plant comprising the steps of: (i) obtaining or generating a construct comprising a selected coding region operably linked to a suppressor of BYDV MP; (ii) transforming a recipient plant cell with said construct; and iii) regenerating a transgenic plant expressing said selected protein from said recipient plant cell. In a specific embodiment, the plant is fertile, and in further embodiments the method further comprises the step of obtaining seed from said fertile transgenic plant. In one aspect, the method further comprises obtaining a progeny plant of any generation from said fertile transgenic plant. In a specific embodiment, a step of transforming comprises a method selected from the group consisting of microprojectile bombardment, PEG mediated transformation of protoplasts, electroporation, silicon carbide fiber mediated transformation, or Agrobacterium-mediated transformation.

In an additional embodiment, there is a method of plant breeding comprising the steps of: (i) obtaining a transgenic plant comprising a selected DNA comprising a suppressor of BYDV MP; and (ii) crossing said transgenic plant with itself or a second plant. In a specific embodiment, the transgenic plant is crossed with said second plant. In another specific embodiment, the second plant is an inbred plant. The method may further comprise the steps of: (iii) collecting seeds resulting from said crossing; (iv) growing said seeds to produce progeny plants; (v) identifying a progeny plant comprising said selected DNA; and (vi) crossing said progeny plant with itself or a third plant. In an additional aspect, the progeny plant inherits said selected DNA through a female parent or through a male parent. In a further aspect, the second plant and the third plant are of the same genotype. In another embodiment, the second and third plants are inbred plants.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows expression of MP inhibits cell proliferation and growth retardation in fission yeast and Arabidopsis thaliana. (A). Inducible expression of MP in fission yeast inhibits cell proliferation (a) and colony formation (b). The BYDV MP gene was expressed under the control of an inducible nmt1 promoter in thiamine-free (MP-on) EMM medium. As a control, MP gene was suppressed in 20 μM thiamine (MP-off) medium. Colony forming ability was examined 5 days after cell streaking on agar plates. (B) Inducible expression of MP in transgenic A. thaliana resulted in retarded growth of the whole plant. Picture was taken 9 days after germination. (C) Inducible expression of MP in transgenic A. thaliana reduced root growth. (a) Expression of MP in a root tip of A. thaliana. Cross section of a root tip was shown (left). Expression of MP is, indicated in root tip by MP::GFP fusion (middle). Overlap of root tip with MP::GFP showed that MP is produced in predominantly in the root stem but not in the root hairs (right). (b) Expression of MP::GFP (middle) but not GFP reduced root growth (left). Measurement of root length 5 and 9 days after germination showed significant reduction of root length due to MP (c).

FIG. 2 demonstrates expression of MP induces cell elongation and cell cycle G2/M arrest in fission yeast and A. thaliana. (A) Cell elongation induced by MP expression in S. pombe. (a) Cell image was captured 24 hr after MP gene induction. (b) Distribution of cell length between MP-suppressing (MP-off) and MP-expressing (MP-on) cells. Cell length was measured individually using an OpenLab software. (c) Effect of MP on cell size and DNA content of S. pombe cells as determined by flow cytometry. Cells were collected 40 hr after gene induction. (top) Forward scatter analysis was used to determine distribution of cell size in a cell population with 1×10⁴ cells. (bottom) cells were first grown under 2.5 μM ammonium chloride as the sole nitrogen source to promote G1 cell population (Alfa et al., 1993). DNA content larger than 2N (G2) indicates possible aneuploidy of fission yeast DNA (see late discussion). (B) A comparison of the cells in the root meristematic regions in the wild type strain (WT) and the transgenic MP strain (TS-4-12). The seeds of the wild type and transgenic strains of Arabidopsis thaliana were germinated on MS media for three days at 4° C. The germinated seedlings were then grown vertically on fresh MS media containing 0.5 μM estradiol at 23° C. in a lighted growth chamber. After three days, the root tips (around 5 mm in length) were collected on ice for confocal microscopy. Each root tip was placed on glass slide with 15 μl of sterile water and was examined using a confocal microscope (Olympus FV500) without further treatment. Major differences were found in the cells in the meristematic zone (top panel) between the wild type and transgenic MP strain (bottom panel). In the wild type strain, the cells in the meristematic zone (indicated by arrows) were compact and their size was regular, whereas in the transgenic MP strain the cells from the same zone (indicated by open arrow heads) were loose and their size was much larger. Bar: 20 μm.

FIG. 3 demonstrates MP-induced cell cycle G2/M arrest by promoting phosphorylation of cyclindependent kinase Cdc2 through Wee1 but not Cdc25. (A) Immunoblot analyses of protein extracts isolated from cells grown in thiamine-plus (MP-off) and thiamine-minus (MP-on) media with anti-Cdc2 and anti-phosphorylated Cdc2. Cells were collected 24 hr after culture. Loading control, an unrelated protein reacting with the antiserum serving as a control for the amount of protein loaded to each lane. (B) Suppression of MP-induced cell elongation by Cdc2-1w mutant, which resists phosphorylation by Wee1. The Cdc2-3w mutant, which promotes de.-phosphorylation of Cdc2 by Cdc25, did not suppress MP-induced cell elongation. Cells were collected 24 hr after culture. (C) MP exerts its cell cycle effect through Wee1 not Cdc25. (a) Suppression of MPinduced cell elongation by a wee1-50 temperature sensitive mutation at permissive (25.5° C.), semi-permissive (30° C.) and non-permissive (36.5° C.) temperatures. Cells were collected 24 hr after cultures. (b) Restoration of colony forming ability on agar plate in MP-expressing cells by wee1-50 mutation. Pictures were taken 5 days after incubation. (c) MP does not inhibit Cdc25 phosphatase activity. A wee1-50 mik1 Δ strain carrying MP in the pYZ1N vector was first grown for 18 hrs at 25° C. in media with or without thiamine and then shifted to 35° C. Measurement of the septation index after the temperature shift allows determination of the Cdc25 phosphatase activity as there are no kinases to phosphorylate Cdc2.

FIG. 4. MP-induced G2/M arrest is independent of DNA damage or replication checkpoints. (A) S. pombe strains that carry mutant for either early checkpoint (Rad3), DNA damage (Chk1), DNA replication (Cds1) or both (Chk1 Cds1) were transformed with MPexpressing plasmid and the effect of MP expression on G2/M arrest was determined by its ability to induce cell elongation. To measure cell length, cell cultures were collected 24 hrs after gene induction. Cell length was measured individually using an OpenLab software as shown in A-a. (A-b) shows distribution of cell length between MPsuppressing (MP-off) and MP-expressing (MP-on) cells. (B) Colony forming ability of checkpoint defective mutants as indicated. Colony forming ability was evaluated 5 days after incubation at 30° C.

FIG. 5 shows suppression of the MP effect by pab1 gene deletion. (A) S. pombe strains that carry a deletion mutant for catalytic (ppa2) or regulatory (pab1) subunits of PP2A were transformed with MP-expressing plasmid and the effect of MP expression on G2/M arrest was determined by its ability to induce cell elongation. To measure cell length, cell cultures were collected 24 hrs after gene induction. Cell length was measured individually using an OpenLab software. (B) Colony forming ability of MP-expressing wild type, ppa2 and pab1 cells. Colony forming ability was evaluated 5 days after incubation at 30° C.

FIG. 6 shows mitotic abnormality caused by MP in S. pombe and A. thaliana. (A) MP causes unequal nuclear segregation that can be suppressed by Δppe1 mutation. (a) S. pombe cells were collected 24 hrs after gene induction and strained with DAPI for observing nuclear morphology. Calcofluor staining was used to visualize cell wall and septum. (i) MP-off wild type cells. (ii-yi) MP-on wild type cells showing unequal chromatids segregation (ii-iii), anuclear cells (iv) and “cut” phenotype (v-yi). (b) normal nuclear segregation in Δppe1 mutant cells. (B) Association of GFP-MP with nucleus. (a) a plasmid carrying gfp-MP was transformed into the wild type S. pombe cells and expressed in thiamine-free medium. Cells were collected 24 his after gene induction. The nuclei were stained with PI. Localization of GFP-MP was detected using a Leica fluorescent microscope with L5 filter with 527/30 (512-542 nm) emission. (b) GFP-MP in Δppe1 mutant cells. Cells were collected and detected in the same way as described in. (a). (C) Genome constitution of the cells from the root tips of the wild type (WT) and transgenic MP strain (TS-4-12) revealed by the detection of 5S rDNA loci using fluorescent in situ hybridization (FISH). (a) Standard control showing normal FISH result with 6 chromosomes. (b) diagram showing locations of different FISH probes. (c) Presence of tetraploid cells in the root tips; from the transgenic, but not the wild type, strains. (i) In the wild type strain the great majority of root tip cells examined were at interphase with six rDNA loci (indicated by arrowheads). Some of those mitotic cells were at metaphase with closely spaced 5S rDNA loci on sister chromatids (ii). Note that the replicated chromosomes aligned regularly along the equatorial plate. In the transgenic strain, about 1% of cells were also found at metaphase (iii). However, the replicated chromosomes distributed irregularly. In about 3 to 4% of cells, 12 rDNA loci were detected (iv). Because these cells were at interphase, they were therefore tetraploid rather than diploid. Bar: 10 μm. (right) The proportion of root tip cells showing abnormal 5S rDNA loci was significantly higher in the transgenic strain than that in the wild type strain. The presence of some cells showing abnormal 5S rDNA loci in the wild type strain may be caused by incomplete adherence of cell materials to the slide during the FISH experiment. (D) Nuclear localization of MP-GFP in root hair cells of A. thaliana.

FIG. 7. Comparative analysis of 5S rDNA loci in the root tip cells from the wild type and transgenic (TS-4-12) plants by FISH. (A) In the wild type plants, the numbers of 5S rDNA loci detected in the root tip cells by FISH were either six (a) or 12 (b). On average, 96% of cells displayed six 5S rDNA loci, they were diploid and were either mitotically inactive or just exited from mitosis. 4% of cells showed 12 5S rDNA loci (B), and they were mainly found in the various phases of the mitotic cell cycle (metaphase, anaphase, telophase). The finding of 4% cells were active in mitotic division in the analysis was consistent with a previous study that showed that the mitotic index in Arabidopsis root tips was between 3% and 3.9% (Hartung et al., 2002). In the transgenic plants, the numbers of 5S rDNA loci detected in the root tip cells by FISH were more variable (A). On average, 80% cells showed six 5S rDNA loci (A, c). 20% of cells showed more than six 5S rDNA loci (B). This cell population was made up of the cells displaying 12 rDNA loci that were in the various stages of the mitotic cell cycle (A, d, 2%), the cells exhibiting 12 loci that were in the interphase (A, e, 3.5%), and the cells showing more than six but less than 10 loci that were also in the interphase (A, f, 14.5%). Reference: Hartung et al., (2002) Current Biology 12: 1787-1791

FIG. 8. Expression of BYDV-GAV MP in wheat and its effect on the growth of wheat plants. In this experiment, the coding sequence of BYDV-GAV MP was cloned into the recombinant genome of barley stripe mosaic virus (BSMV), which has previously been found to be infectious in wheat plants (Edwards, 1995). (A) A diagram showing the tripartite RNA genome of BSMV (α, β, and γ) and the modified RNAγ molecules that were used to express the BYDV-GAV MP::γb fusion protein or the γb::GFP fusion protein in wheat plants. The five types of RNA molecules were produced by in vitro transcription from their respective DNA clones. Combined inoculation of RNAα, RNAβ and RNAγ (MP::γb) would give rise to MP::γb fusion protein, whereas the inoculation of RNAα, RNAβ and RNAγ (γb::GFP) would produce γb::GFP fusion protein, which would be employed as a control to assess the effect of MP::γb fusion protein on the growth of wheat plants. (B) Four weeks after inoculation, the growth of the plants expressing MP::γb was much more severely inhibited compared to that of the plants expressing γb::GFP or the uninoculated (CK) plants. (C) The height of the plants expressing MP::γb was significantly reduced compared to that of the plants expressing γb::GFP or the uninoculated (CK) plants (n=30). (D) RT-PCR experiments confirmed the transcription of the MP::γb coding sequence in the five individual plants expressing MP::γb fusion protein but not in the uninoculated (CK) plants.

### DETAILED DESCRIPTION OF THE INVENTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

### I. Definitions

The term “DNA construct” as used herein refers to DNA, such as in a vector, having a promoter operably linked to a DNA polynucleotide. DNA construct may be used interchangeably with the terms DNA polynucleotide and DNA vector.

The term “promoter” as used herein refers to a regulatory region of DNA, which may comprise a TATA box, that is capable of directing RNA polymerase II to initiate RNA synthesis at the appropriate transcription initiation site for a particular coding sequence. A promoter may additionally comprise other recognition sequences, for example, generally positioned upstream or 5′ to the TATA box, referred to as upstream promoter elements, which may influence the transcription initiation rate, for example.

The term “operably linked” as used herein refers to a functional linkage between a promoter and a second sequence, wherein the promoter sequence initiates and mediates transcription of the DNA sequence corresponding to the second sequence. In specific embodiments, operably linked means that the nucleic acid sequences being linked are contiguous where necessary to join two protein-coding regions in the same reading frame.

The term “DNA encoding BYDV movement protein (MP)” as used herein refers to a DNA polynucleotide having, comprising, consisting of, or consisting essentially of SEQ ID NO:2, or any portion, fragment, or variant thereof, as well as wholly or partially synthesized polynucleotides. It will be appreciated by those of ordinary skill in the art that, as a result of the degeneracy of the genetic code, there are many nucleotide sequences that encode a peptide as described herein. Some of these polynucleotides bear minimal homology to the nucleotide sequence of any native gene, in specific embodiments. Nonetheless, polynucleotides that vary due to differences in codon usage are specifically contemplated by the present invention.

The term “Movement Protein (MP) variants” as used herein refers to polynucleotides that may contain one or more substitutions, additions, deletions, and/or insertions such that the activity or antigenic properties of the peptides encoded by the variants are not substantially diminished, relative to the corresponding MP. Such modifications may be readily introduced using standard mutagenesis techniques, such as oligonucleotide directed site-specific mutagenesis as taught, for example, by Adelman et al. (DNA, 2:183, 1983). Variants may also include what may be referred to as “fragments.” In specific embodiments, the fragments have activities of movement proteins. In certain embodiments of the variants, a MP nucleic acid variant comprises no more than about 10 alterations compared to SEQ ID NO:2, wherein the alterations may be deletions, substitutions, and/or inversions. In particular aspects, the fragments may be at least 70% identical over their length to SEQ ID NO:2, at least 75% identical over their length to SEQ ID NO:2, at least 80% identical over their length to SEQ ID NO:2, at least 85% identical over their length to SEQ ID NO:2, at least 90% identical over their length to SEQ ID NO:2, at least 95% identical over their length to SEQ ID NO:2, and so forth. In certain aspects, the fragments encode a N-terminal alpha helix. The fragments may hybridize under stringent conditions to SEQ ID NO:2, in certain aspects. In particular aspects, the fragments are at least about 100 contiguous nucleotides of SEQ ID NO:2, at least about 150 contiguous nucleotides of SEQ ID NO:2, at least about 175 contiguous nucleotides of SEQ ID NO:2, at least about 200 contiguous nucleotides of SEQ ID NO:2, at least about 225 contiguous nucleotides of SEQ ID NO:2, at least about 250 contiguous nucleotides of SEQ ID NO:2, at least about 275 contiguous nucleotides of SEQ ID NO:2, at least about 300 contiguous nucleotides of SEQ ID NO:2, at least about 325 contiguous nucleotides of SEQ ID NO:2, at least about 350 contiguous nucleotides of SEQ ID NO:2, at least about 400 contiguous nucleotides of SEQ ID NO:2, at least about 450 contiguous nucleotides of SEQ ID NO:2, at least about 500 contiguous nucleotides of SEQ ID NO:2, at least about 550 contiguous nucleotides of SEQ ID NO:2, at least about 600 contiguous nucleotides of SEQ ID NO:2, at least about 650 contiguous nucleotides of SEQ ID NO:2, at least about 700 contiguous nucleotides of SEQ ID NO:2, at least about 750 contiguous nucleotides of SEQ ID NO:2, at least about 800 contiguous nucleotides of SEQ ID NO:2, at least about 850 contiguous nucleotides of SEQ ID NO:2, and so forth.

In MP amino acid variants, the variants may comprise one or more substitutions of amino acids, such as compared to SEQ ID NO:1, for example. In specific embodiments, the amino acid variants comprise one or more conservative amino acid substitutions compared to SEQ ID NO:1. The variants may comprise 1, 2, 3, 4, 5, 6, 7, 8, 9, 10, or more substitution; compared to SEQ ID NO:1.

The term “Viricide” as used herein refers to an agent (physical or chemical, for example) that inactivates or destroys viruses.

The term “anti-sense RNA” as used herein, refers to an RNA molecule that is capable of forming a duplex with another polynucleotide, such as a second RNA molecule. Thus a given RNA molecule is said to be an anti-sense RNA molecule with respect to a second, complementary or partially complementary RNA molecule, i.e., the target molecule. An anti-sense RNA molecule may be complementary to a translated or an untranslated region of a target RNA molecule. The anti-sense RNA need not be perfectly complementary to the target RNA. Anti-sense RNA may or may not be the same length of the target molecule; the anti-sense RNA molecule may be either longer or shorter than the target molecule.

The term “co-suppressor RNA” refers to an RNA molecule that effects suppression of expression of a target gene where the RNA is partially homologous to an RNA molecule transcribed from the target gene. A co-suppressor RNA molecule is the RNA molecule that effects co-suppression as described in U.S. Pat. No. 5,231,020, Krol et al., Biotechniques 6:958-976 (1988), Mol et al., FEBS Lett. 268:427-430 (1990), and Grierson, et al, Trends in Biotech. 9:122-123 (1991) and similar publications, which references are incorporated by reference as if set forth herein in their entirety. A “cosuppressor” RNA is in the sense orientation with respect to the target gene, i.e., the opposite orientation of the anti-sense orientation.

The term “inhibitory RNA encoding polynucleotide” as used herein, refers to a polynucleotide, e.g., DNA, RNA, and the like, capable of being transcribed, when in functional combination with a promoter, so as to produce an inhibitory RNA molecule that interferes with the expression of a target gene, e.g., an anti-sense RNA or a cosuppressor RNA. Anti-sense RNA-encoding polynucleotides and co-suppressor encoding polynucleotides are both embodiments of the inhibitory RNA encoding polynucleotides. When the inhibitory RNA is an anti-sense RNA, the inhibitory RNA transcribed from the inhibitory RNA encoding polynucleotide region of the DNA constructs of the invention is preferably substantially complementary to the entire length of the RNA molecule or molecules for which the anti-sense RNA is specific, i.e., the target. Total complementarity, however, is not required, in specific embodiments. In particular aspects, it is sufficient that the inhibitory RNA complex or bind with the target and reduce its expression. The anti-sense RNA encoding polynucleotide in the subject vectors may encode an anti-sense RNA that forms a duplex with a non-translated region of an RNA transcript such as an intron region, or 5′ untranslated region, a 3′ untranslated region, and the like. Similarly, a co-suppressor encoding polynucleotide in the subject vectors may encode an RNA that is homologous to translated or untranslated portions of a target RNA. Anti-sense RNA encoding polynucleotides may be conveniently produced by using the non-coding strand, or a portion thereof, of a DNA sequence encoding a protein of interest. Exemplary BYDV MP amino acid (SEQ ID NO:1) and DNA sequences (SEQ ID NO. 2) are provided.

The term “reduced expression,” as used herein, is a relative term that refers to the level of expression of a given gene in a cell produced or modified by the claimed methods as compared with a control, which is a comparable unmodified cell, i.e., a cell lacking the subject vector, under a similar set of environmental conditions. Thus, a cell modified by the subject methods, i.e., a cell having “reduced expression” of the gene of interest, may express higher levels of that gene under a first set of environmental conditions, than a comparable unmodified cell under a second set of environmental conditions, such as when the second set of conditions is highly favorable to gene expression.

The term “inducible” as applied to a promoter is well understood by those skilled in the art. In essence, expression under the control of an inducible promoter is “switched on” or increased in response to at least one applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression occurs in the absence of the stimulus, expression from any inducible promoter is increased in the presence of at least one correct stimulus. In specific embodiments, the level of expression increases upon application of the relevant stimulus by an amount effective to alter a phenotypic characteristic. Thus, an inducible (or “switchable”) promoter may be used that causes a basic level of expression in the absence of the stimulus, wherein the level is too low to bring about a desired phenotype (and may in fact be zero or undetectable). Upon application of the stimulus, expression is increased (or switched on) to a level that brings about the desired phenotype. Inducible promoters may be advantageous in certain circumstances because they place the timing of reduction in expression of the target gene of interest under the control of the user.

### II. Embodiments of the Invention

It has been discovered that barley yellow dwarf virus (BYDV) viral movement protein (referred to as “MP” or, interchangeably, “BYDV MP”) contributes significantly to retarded growth in BYDV-infected plants. MP is encoded by BYDV gene P4, and an exemplary embodiment is provided herein as SEQ ID NO:1. Experiments were conducted on the well known plant model Arabidopsis thaliana, a small cruciferous plant; tobacco; wheat (host plant); and in a new fission yeast model using Schizosaccharomyces pombe. It was discovered that expression of MP in both BYDV-infected plants and S. pombe fission yeast cells inhibits cell proliferation and causes gross enlargement and elongation of the cells, in specific embodiments by MP-induced cell cycle G2/M arrest. Transgenic TS-4-12 plants that express the MP gene show the primary symptoms of BYDV infections that are stunting and discoloration of the leaf tips such as yellowing, reddening or purpling. Based on the discovery that MP contributes significantly to the BYDV-disease phenotype, certain embodiments of the present invention are directed to an in vitro method for using BYDV MP or a variant thereof to screen agents for the ability to inhibit and/or down-regulate expression of MP. Such agents are candidate MP suppressors. This is accomplished by contacting BYDV MP or an MP variant either directly or indirectly with a test agent under conditions suitable to allow complex formation. If a complex is detected, then the test agent is an agent that binds to BYDV MP or the MP variant. The agent is selected as a candidate MP suppressor, which may then be further tested to determine whether the agent suppresses BYDV MP expression and/or activity in infected cells, for example using the in vivo screening method below.

Another embodiment is directed to an in vivo screening assay using yeast cells transformed to express BYDV MP. In this embodiment, test agents are screened in vivo for the ability to suppress BYDV MP expression or activity, such as by the following exemplary method: establishing, a control yeast cell culture and a test yeast cell culture such that the yeast cells in the control and test cultures have a copy of an inducible BYDV MP gene or an inducible variant either integrated or episomal thereof; contacting the test yeast cell culture with the test agent; (b) growing the control and test cultures under conditions suitable to induce expression of the BYDV MP gene or MP variant; and screening the test cultures for a cell characteristic and/or phenotype not present in the control yeast cell culture. The presence of the cell characteristic and/or cell phenotype in the test culture indicates that the test agent is a suppressor of BYDV MP expression or activity. The characteristic or phenotype includes at least one of BYDV-MP-induced cell death, the inability to form colonyforming units (CFU), abnormal cell enlargement or elongation, and/or G2 arrest.

Another embodiment is directed to the BYDV MP suppressor identified using a screening method of the invention, such as the exemplary in vivo screening method above, for example to its use as a viricide to protect a plant at risk for infection by BYDV and/or to decrease the adverse side effects of a BYDV infection. Another embodiment is directed to a plant cell or a yeast cell transformed with an expression vector comprising DNA encoding BYDV MP or a MP variant thereof, and the regulatory elements necessary to express the DNA in the plant or yeast cell. Such transformed cells are useful in at least some screening assays of the present invention. Other embodiments are directed to expression vectors comprising the DNA encoding BYDV MP or a MP variant thereof, and the regulatory elements necessary to express the DNA in the plant or yeast cell for transforming plant or yeast cells or any eukaryotic cell to express BYDV MP.

Another embodiment of the present invention is directed to expression vectors comprising DNA encoding the MP suppressors identified in the exemplary screening methods of the present invention and, optionally, any regulatory elements necessary to express the DNA in a eukaryotic cell, including, a plant or yeast cell. The vector may comprise a constitutive or inducible promoter, such as, for example, a promoter induced in the presence of muristerone A. Regulatory elements are known and standard in the art.

Construction of vectors is well known in the art using standard molecular biological techniques. Representative examples of yeast useful in this embodiment include but are not limited to a fission yeast or a budding yeast. In specific embodiments, the fission yeast is Schizosaccharomyces pombe. In other specific embodiments, the budding yeast is Saccharomyces cerevisiae. In certain embodiments, the vector is a plasmid comprising DNA encoding the suppressors and regulatory elements sufficient to express the suppressor protein.

One of the primary goals of genetic engineering has been to control the expression of selected genes in eukaryotic organisms of interest. One method of reducing the expression of specific genes in eukaryotic organisms has been through the use of antisense RNA and co-suppressor RNA. Anti-sense RNA has been used to reduce the expression of pre-selected genes in both plants and animals. Based on the discovery that BYDV MP contributes to the disease phenotype in plants, the invention is further directed to the use of inhibitory anti-sense RNA that hybridizes with BYDV MP mRNA or DNA to reduce expression of MP in infected plants. Thus, an embodiment is directed to DNA constructs for the expression of inhibitory RNA in the cytoplasm of eukaryotic cells, especially plant cells, which RNA will down-regulate the transcription, expression or translation of BYDV MP. The DNA constructs/vectors of the invention are capable of replicating in the cytoplasm of a eukaryotic plant cell and comprise a promoter region in functional combination with an anti-sense RNA or a co-suppressor RNA that inhibits BYDV movement protein, thereby interfering with its expression and activity. In order to down-regulate expression of BYDV MP, the inhibitory RNA sequence will comprise sufficient homology or sequence identity to the target sequence encoding BYDV MP to down-regulate its expression.

The amount of sequence homology needed to downregulate target gene expression is known in the art of anti-sense, interference RNA or related technologies and is described in more detail in, for example, U.S. Pat. Nos. 6,635,805 and 6,376,752 which are both incorporated herein in their entirety.

Yet another embodiment of the invention is directed to a DNA construct comprising DNA encoding one or more BYDV MP suppressors identified using the exemplary screening assays above, to transform a plant, yeast or other eukaryotic cell, thereby conferring on the cell resistance to the adverse effects of BYDV infection. Transgenic plants expressing one or more suppressors of BYDV MP expression or activity also are encompassed by the scope of the present invention. Certain embodiments of the DNA constructs of this invention may be designed so as to replicate in the cytoplasm of plant cells or yeast cells. When the eukaryotic cell of interest is a plant cell, the genetic construction is preferably derived from a plant RNA virus, more preferably a positive single-stranded RNA virus. Plant RNA virus-derived DNA constructs may comprise a plant virus subgenomic promoter, including subgenomic promoters from tobamoviruses, in functional combination with the inhibitory RNA encoding region, for example.

Certain other embodiments are directed to plant or yeast cells transformed to express interfering RNA that hybridizes with and/or interacts with the DNA encoding BYDV MP or, alternatively, to mRNA encoding BYDV MP. BYDV MP transcription, or translation is downregulated by the interfering RNA, thus making transgenic plant or yeast cells BYDV MP-resistant. Certain embodiments are directed to transgenic plants that have a plurality of such transformed cells expressing interfering RNA. Another embodiment is directed to plants that have cells that have been transformed to express one or more BYDV MP suppressors identified using the screening methods of the present invention, which suppressor(s) confer resistance to BYDV infection by interfering with BYDV MP expression and activity.

Further embodiments are directed to methods of generating transgenic plants resistant to barley yellow dwarf virus (BYDV) infection, wherein the plant comprises at least one plant cell expressing inhibitory anti-sense RNA that hybridizes with the gene or, alternatively, mRNA encoding BYDV MP or, alternatively, co-suppressor RNA that down-regulates BYDV MP expression. The plant cells may be produced by transfecting with a viral vector capable of replication in the plant cell, which vector has a promoter in functional combination with a polynucleotide that encodes the inhibitory RNA. In one embodiment the promoter is a plant viral RNA promoter, for example; in another embodiment at least one of the promoters is derived from a tobamovirus, for example. In one embodiment, the viral vector is obtained from a positive single-stranded RNA virus that is optionally a tobamovirus, such as tobacco mosaic virus, for example.

### III. Brief Discussion of the Examples

BDV is a single (+) strand RNA luteovirus. It has a small genome with 5,673 kilobases (kb), which encodes one virion protein and six non-virion proteins (P1-P6). Using fission yeast as a model system, the inventors have characterized each of these BYDV open reading frames (ORF) and have discovered that one of the BYDV nonviral proteins identified in the literature as movement protein (MP) (encoded by the P4 gene), is the viral determinant that is responsible for growth retardation of BYDV-infected plants. BYDV MP has a role in the nuclear transport of viral RNA. However, its pathological role in contributing to retardation of the plant was not known before being described herein.

The well known plant model Arabidopsis thaliana, a small cruciferous plant, was used to conduct certain experiments described herein. A fission yeast (Schizosaccharomyces pombe) model system was utilized to perform initial functional screening of BYDV gene products. Fission yeast is a single cell eukaryotic organism that possesses many of the same molecular and biochemical characteristics as higher eukayotes [For review of this subject, see (Zhao and Lieberman, 1995), for example]. Use of fission yeast as a model organism in studying gene expression and function in eukaryotes, including viral gene function, has been demonstrated previously (Lee and Nurse, 1987; Zhao et al., 1996). Importantly, fission yeast, like plants, has a cell wall, thus making it a suitable genetically tractable system to study plant-related genes. Example 1 describes preparation of the yeast and plant model systems in detail.

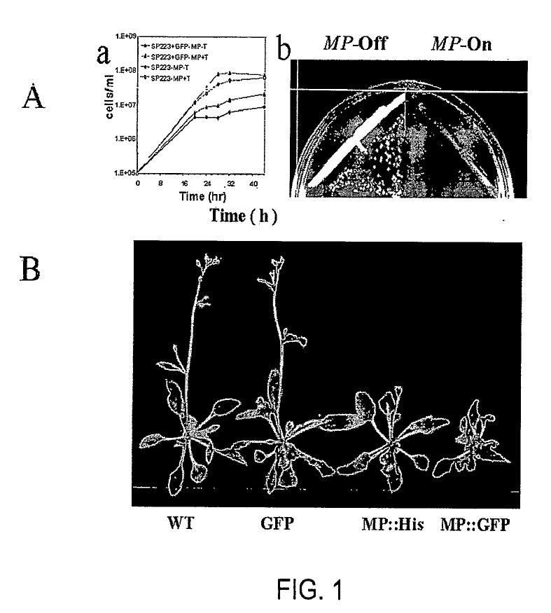

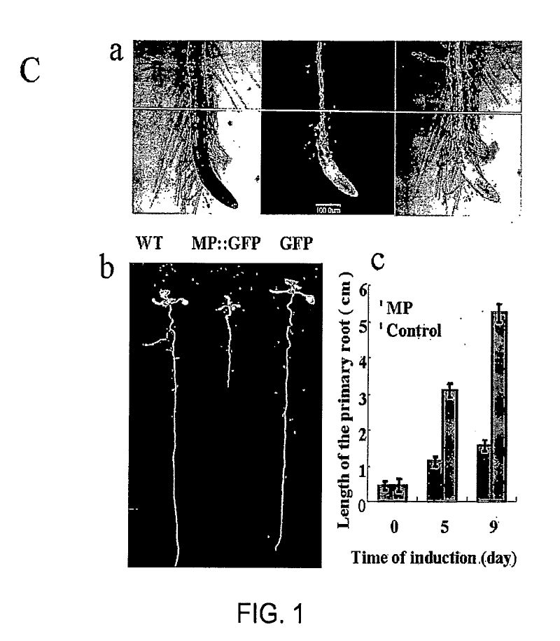

Example 2 shows that expression of BYDV MP inhibits cell proliferation and causes growth retardation in both S. pombe and A. thaliana. More than one log difference in cell growth was observed between the MP-induced and MP-repressed fission yeast cells (FIG. 1A-a). Consistently, little or no colony formation was observed on agar plates when the MP gene was expressed (FIG. 1A-b, right). By contrast, normal colony formation was observed when the MP gene was suppressed by plating cells on thiamine-containing agar plate (FIG. 1A-b, left). Inducible expression of MP in transgenic A. thaliana also significantly retarded growth of the whole plant (FIG. 1B) and reduced root growth (FIG. 1C).

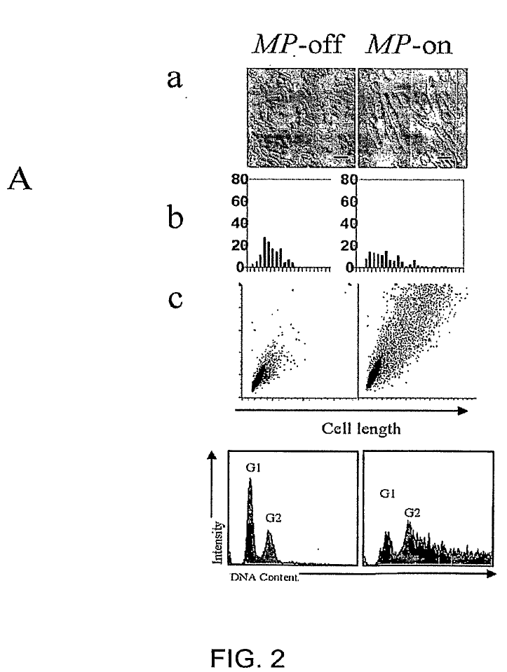

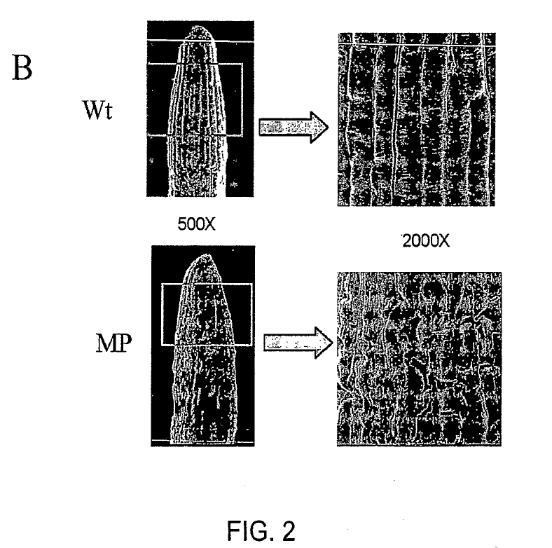

Example 3 shows experiments testing the effect of MP on cell morphology of fission yeast cells. In the thiamine-containing growth medium (MP gene expression is OFF; FIG. 2A, left), fission yeast cells with MP plasmid are of normal length [10.4±0.2μ; (Zhao and Lieberman, 1995)]. In contrast, the mean cell length of the MP-expressing strain is 12.6±0.4μ (standard error of the mean) and is statistically significant at the p<0.0001 level when compared to cell length of the wild-type (FIG. 2A-a). In addition to an increased mean cell length in the MP-expressing cell population, some of the cells are longer than 18μ, whereas no such cells are seen in the MP-repressed cells (FIG. 2A-b). Example 3 also shows a comparison of the morphology in the root meristematic regions in the wild type and the MP-transgenic strains (FIG. 21) that revealed major differences. In the wild type strain, the cells in the meristematic zone were compact and their size was regular, whereas cells in the transgenic strain from the same zone were longer and larger (FIG. 2B, right). Cell cycle G2/M transition is a highly regulated cellular process, in which the cyclindependent kinase Cdc2 plays a pivotal role. In all eukaryotes, progression of cells from G2 phase of the cell cycle to mitosis requires activation of Cdc2 (Morgan, 1995).

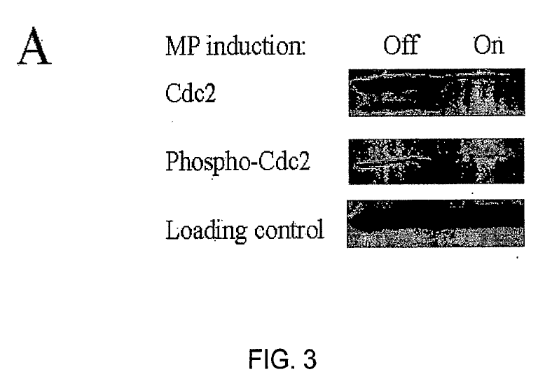

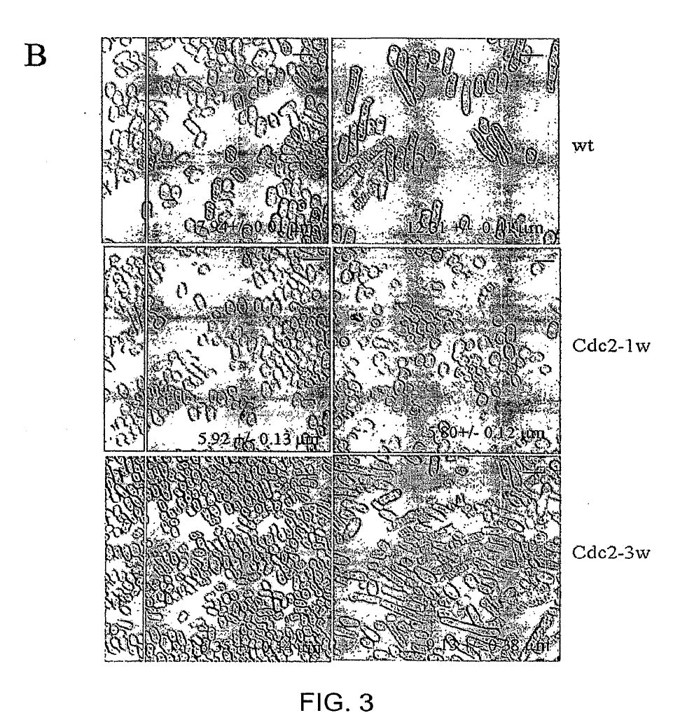

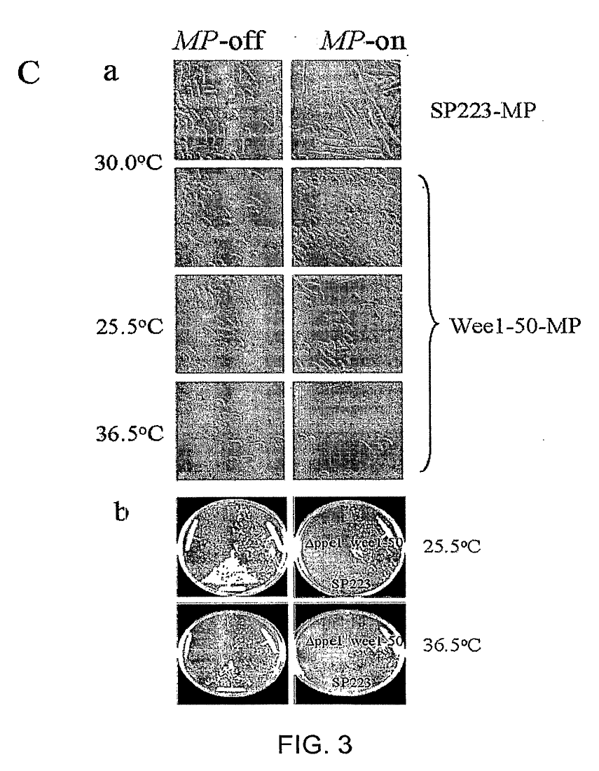

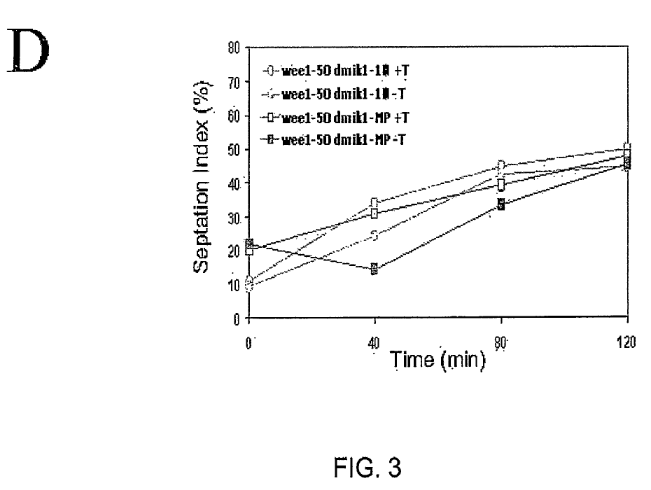

Typically, entry to mitosis is regulated by phosphorylation status of Cdc2, which is phosphorylated by Wee1 kinase during G2 and rapidly dephosphorylated by the Cdc25 phosphatase to trigger entry to mitosis (Gould and Nurse, 1989; Krek and Nigg, 1991; Morgan, 1995; Norbury et al., 1991). To determine whether MP exerts its cell cycle effect directly on Cdc2, phosphorylation status of Cdc2 kinase was measured under the MP-on and MP-off conditions using immunoblot analyses (FIG. 3A). Example 4 shows that MP induces cell cycle G2/M arrest by impinging upon the mitotic determinant Cdc2 through Wee1 kinase, but not through Cdc25 phosphatase. Notably, MP induces G2/M arrest by modulating a mechanism involving protein phosphatase 2A (PP2A)-related proteins, rather than the classic phosphatase checkpoints.

Example 5 describes experiments showing that MP does not use the DNA damage and DNA replication checkpoints during induction of G2/M arrest. MP and the checkpoints for DNA damage and DNA replication all induce cell cycle arrest through phosphorylation of Tyr15 on Cdc2 (Chen et al., 2000; Nurse, 1997; Rhind and Russell, 1998). To test whether MP might induce G2 arrest through one of the checkpoint pathways, MP was expressed in a several strains having mutations causing defects in the early or late steps of the checkpoint pathways. None of the mutations significantly reduced MP-induced G2 arrest, indicating that MP must use an alternative pathway to induce G2 arrest.

Previous studies demonstrated that other viral proteins such as HIV-1 Vpr or adenovirus E4Orf4 induces cell cycle G2 arrest by modulation through PP2A (Elder et al., 2000; Kornitzer et al., 2001). Studies described in Example 6 indicate that MP functionally interact with PP2A-like enzyme during induction of cell cycle arrest, in certain embodiments.

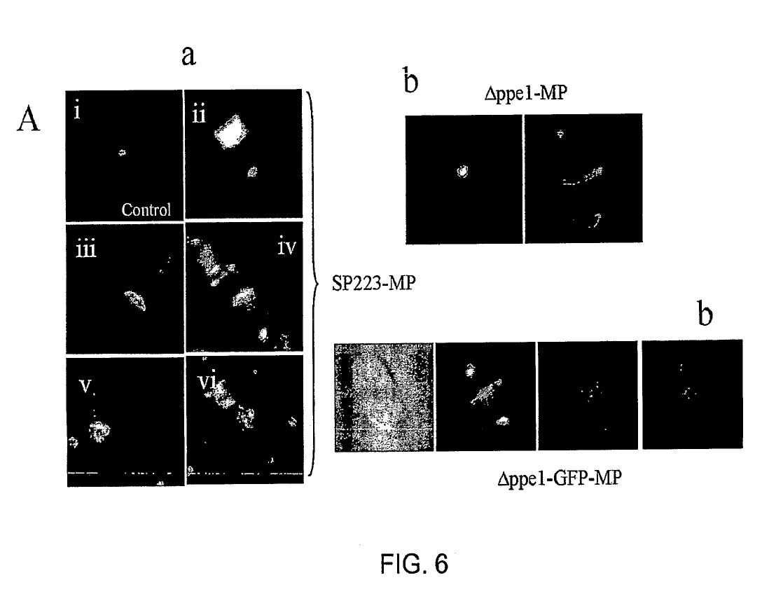

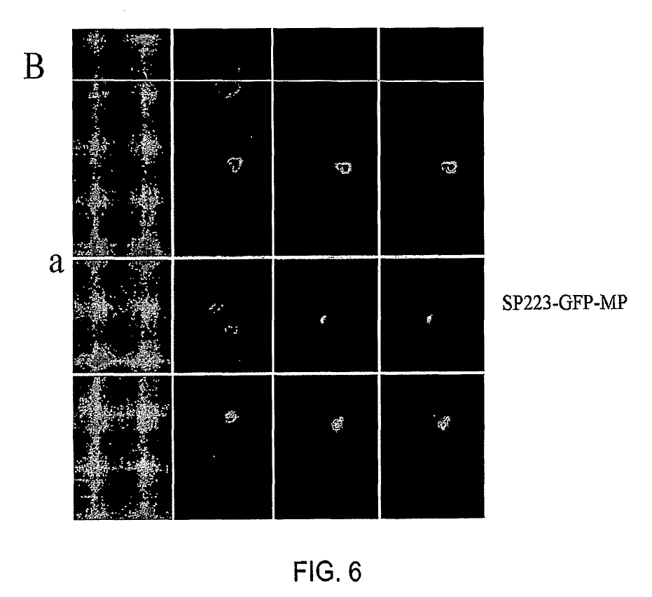

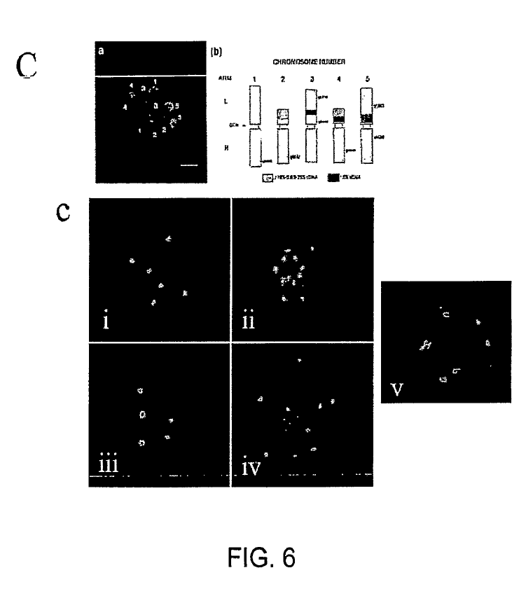

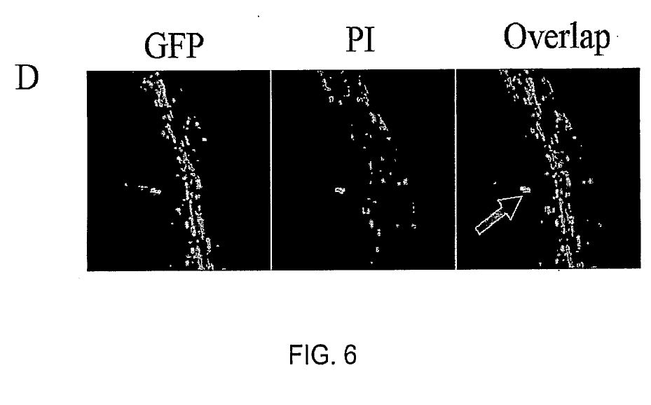

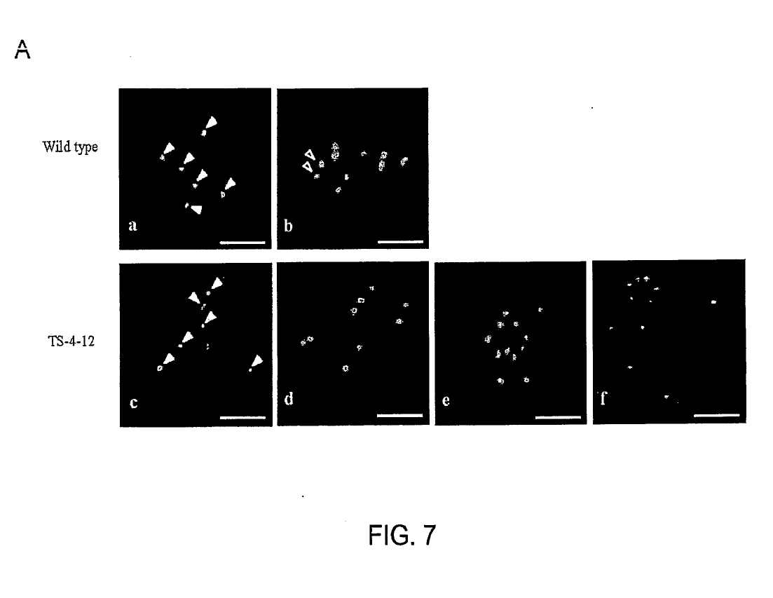

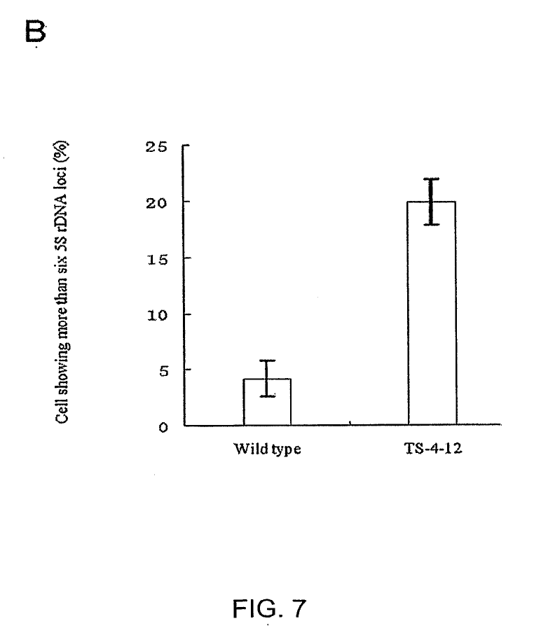

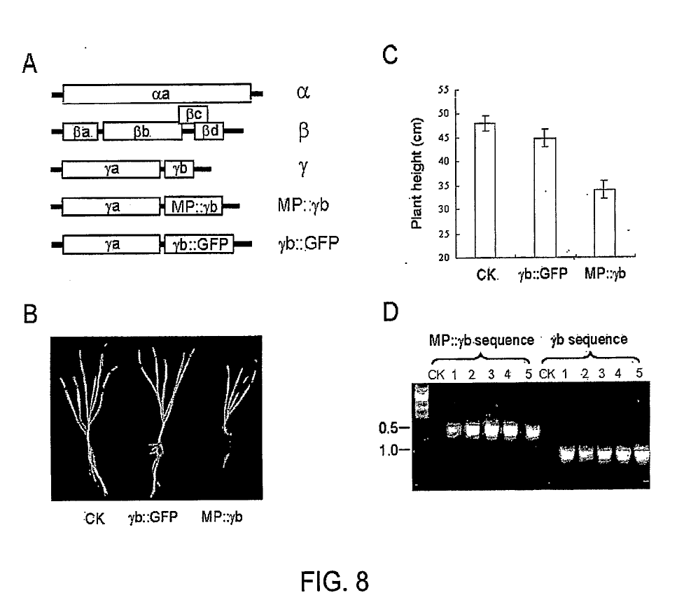

The studies in Example 7 show that normal nuclear morphology with equal segregation was observed in MP-repressed cells after septum formation (FIG. 6A-a-i). By contrast, MP-expressing cells showed significant mitotic abnormality including unequal chromosome segregation (FIG. 6A-b-ii-iv) and “cut” phenotype [FIG. 6A-b-v-vi; (Funabiki et al., 1996)]. Additional experiments showed that MP may have added impact on mitosis. To determine whether MP has similar effect on chromosome segregation in plant cells, the chromosomal ploidy of root hair cells was determined using fluorescence in situ hybridization (FISH). To quantify potential differences of abnormal mitotic cells between the wild type and MP-transgenic plants, the proportion of root tip cells showing abnormal 5S rDNA loci was measured. As shown in FIG. 7B, the percentage of abnormal 5S rDNA loci was significantly higher in the transgenic strain than that in the wild type strain.

### IV. Screening For Modulators of the Protein Function

In certain embodiments, the present invention comprises methods for identifying suppressors of BYDV MP, including the function, production, or expression of MP. These assays may comprise random screening of large libraries of candidate substances; alternatively, the assays may be used to focus on particular classes of compounds selected with an eye towards structural attributes that are believed to make them more likely to modulate the function, production of, or expression. By function, it is meant that one may assay for one or more phenotypes associated with BYDV MP, including, for example, a change in cell proliferation, change in viability in culture, cell size change, cell length change, cell cycle G2 arrest, and so forth.

To identify a BYDV MP suppressor, one generally will determine the function of BYDV MP in the presence and absence of the candidate substance, a suppressor defined as any substance that suppresses function at least in part. For example, a method generally comprises:

(a) providing a candidate suppressor (which may also be referred to as a test agent);

(b) admixing the candidate suppressor with an isolated compound or cell, or a suitable experimental animal;

(c) measuring one or more characteristics of the compound, cell or animal in step (c); and

(d) comparing the characteristic measured in step (c) with the characteristic of the compound, cell or animal in the absence of said candidate suppressor,

wherein a difference between the measured characteristics indicates that said candidate suppressor is, indeed, a suppressor of the compound, cell or animal.

Assays may be conducted in cell free systems, in isolated cells, or in organisms including transgenic animals. It will, of course, be understood that all the screening methods of the present invention are useful in themselves notwithstanding the fact that effective candidates may not be found. The invention provides methods for screening for such candidates, not solely methods of finding them.

1. Modulators

As used herein the term “candidate substance” refers to any molecule that may potentially inhibit BYDV MP activity. The candidate substance may be a protein or fragment thereof, a small molecule, or even a nucleic acid molecule, for example. It may prove to be the case that the most useful pharmacological compounds will be compounds that are structurally related to a related molecule. Using lead compounds to help develop improved compounds is know as “rational drug design” and includes not only comparisons with known inhibitors and activators, but predictions relating to the structure of target molecules.

The goal of rational drug design is to produce structural analogs of biologically active polypeptides or target compounds. By generating such analogs, it is possible to fashion drugs, which are more active or stable than the natural molecules, that have different susceptibility to alteration or that may affect the function of various other molecules. In one approach, one would generate a three-dimensional structure for a target molecule, or a fragment thereof. This could be accomplished by x-ray crystallography, computer modeling, or by a combination of both approaches, for example.

It also is possible to use antibodies to ascertain the structure of a target compound inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, for example, would be ideal candidate inhibitors.

In addition to the modulating compounds initially identified, the inventors also contemplate that other sterically-similar compounds may be formulated to mimic the key portions of the structure of the modulators. Such compounds, which may include peptidomimetics of peptide modulators, may be used in the same manner as the initial modulators.

An inhibitor according to the present invention may be one that exerts its inhibitory effect upstream, downstream, or directly on BYDV MP. Regardless of the type of inhibitor identified by the present screening methods, the effect of the inhibition by such a compound results in a change in phenotype as compared to that observed in the absence of the added candidate substance.

2. In vitro Assays

A quick, inexpensive and easy assay to run is an in vitro assay. Such assays generally use isolated molecules, can be run quickly and in large numbers, thereby increasing the amount of information obtainable in a short period of time A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

3. In cyto Assays

The present invention also contemplates the screening of compounds for their ability to modulate BYDV MP in cells. Various cell lines can be utilized for such screening assays, including cells specifically engineered for this purpose. For example, a cell may comprise a BYDV MP nucleic acid operably linked to a heterologous promoter, such as an inducible promoter for example. The cell may also comprise a reporter gene operably linked to a heterologous promoter, such as an inducible promoter, for example.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

4. In vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

The present invention provides methods of screening for a candidate substance that inhibits BYDV MP. In these embodiments, the present invention is directed to a method for determining the ability of a candidate substance to inhibit BYDV MP, generally including the steps of: administering a candidate substance to the animal; and determining the ability of the candidate substance to reduce one or more characteristics of BYDV infection.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

### V. Suppressors of BYDV MP

In certain aspects of the invention, there are one or more suppressors of BYDV MP. The suppressors may be of any suitable kind, but in specific embodiments, they comprise nucleic acid, protein, peptide, polypeptide, small molecule, mixtures thereof, combinations thereof, and so forth. Nucleic acids may be RNA or DNA. The suppressors may comprise inhibitory RNA, for example, small inhibitory RNA (microRNA & RNAi).

Small molecule libraries for use in screening methods of the invention may be of any suitable kind. Exemplary sources for libraries to employ include, for example, the Molecular Libraries Screening Center Network (available through the National Institutes of Health) or Ligand.Info, which is a compilation of various publicly available databases of small molecules.

In certain aspects of the invention, a nucleic acid is employed to inhibit BYDV MP. In certain aspects, the nucleic acid is an inhibitory RNA, such as a siRNA, for example. The inhibitory RNA may be directed to any region of the gene, including, for example, one or more of a 5′ leader sequence, an exon, an intron, a splice junction, or a 3′ UTR. The inhibitory RNA may be of any suitable length, but in particular aspects it is at least about 10, 15, 20, 25, 30, 35, 40, 45, 50, 55, 60, 65, 70, 75, 100, 110, 120, 130, 140, 150, 160, 170, 180, 190, 200, 210, 225, 250, 275, 300, 325, 350, 375, 400, 425, 450, 475, 500, 525, 550, 575, 600, 625, 650, 675, 700, 725, 750, 775, 880, 825, 850, or 875 or more in length. In specific aspects of the invention, the inhibitory RNA has 100% sequence identity to the corresponding target sequence in a BYDV MP polynucleotide (such as a BYDV MP gene or BYDV MP mRNA) (in specific embodiments may be the exemplary sequence of SEQ ID NO:2). However, in specific embodiments, the inhibitory RNA is at least about 70%, at least about 75%, at least about 80%, at least about 85%, at least about 90%, at least about 95%, at least about 97%, or at least about 99% identical to the corresponding target sequence in a BYDV MP. One of skill in the art recognizes that a siRNA molecule is the complement of a corresponding transcribed BYDV MP sequence and that a cosuppressor RNA corresponds to the transcribed sequence.

### VI. Exemplary Transgenic Plants Resistant to BYDV Infection

Although in specific embodiments transgenic plants of the invention comprise one or more cells that produce BYDV MP, such as for use in an in vivo screening method, in particular aspects provide a transgenic plant that comprises one or more cells that produce a molecule that renders the cell and organism resistant to BYDV infection. In specific embodiments, such as molecule comprises inhibitory RNA.

Descriptions of the use of anti-sense RNA to reduce the expression of selected genes in plants can be found at least in U.S. Pat. No. 5,107,065, Smith et al., Nature 334:724-726 (1988), Van der Krol et al., Nature 333:866-869 (1988), Rothstein et al., Proc. Natl. Acad. Sci. USA 84:8439-8443 (1987), Bird et al., Bio/Technology 9:635-639 (1991), Bartley et al., Biol. Chem. 267:5036-5039 (1992), and Gray et al., Plant Mol. Bio. 19:69-87 (1992) which references are all incorporated by reference as if set forth herein in its entirety. Co-suppressor RNA, is in the same orientation as the RNA transcribed from the target gene, i.e., the “sense” orientation. Methods for transforming plant cells with anti-sense RNA and co-suppressor RNA are described at least in U.S. Pat. No. 6,376,752, which reference is incorporated by reference as if set forth herein in its entirety.

Although the expression of numerous genes in transgenic plants has been repressed by anti-sense RNA, the actual mechanism and location of inhibition is not known. Antisense RNA may directly interfere with transcription of DNA in the nucleus or form duplexes with the heterogeneous nuclear (hnRNA). There is evidence that inhibition of endogenous genes occurs in transgenic plants containing sense RNA, A. R. van der Krol et al., Nature 333:866-869 (1988) and C. Napoli et al., Plant Cell 2:279-289 (1990). The mechanism of this down regulation or “co-suppression” is thought to be caused by the production of anti-sense RNA by read through transcription from distal promoters located on the opposite strand of the chromosomal DNA (Greison, et al. Trends in Biotech. 9:122-123 (1991)). Alternatively, in the cytoplasm, anti-sense RNA may form a double-stranded molecule with the complimentary mRNA, thus preventing the translation of mRNA into protein. It has been shown by others that RNA can reduce the expression of a target gene through inhibitory RNA interactions with target mRNA that take place in the cytoplasm of a eukaryotic cell, rather than in the nucleus (see, for example, U.S. Pat. No. 6,376,752).

Thus, anti-sense RNA and co-suppressor RNA expressed in the cytoplasm are effective inhibitors or down-regulators of expression of a target protein. In the context of the present invention, anti-sense RNA, including interfering RNA or related technologies and co-suppressor RNA, are considered inhibitory RNAs. Cytoplasmic expression of inhibitory RNA (specific for target genes such as BYDV MP) has numerous advantages over nuclear expression, these advantages include the ability to use high level expression vectors that are not suitable for nuclear expression. The use of such vectors is particularly advantageous in plants, because vectors capable of systemically infecting plants may be used to produce the inhibitory RNA (see, for example, U.S. Pat. No. 6,376,752).

The vectors for transformation of plant cells may be of any suitable kind but in specific embodiments they are derived from RNA plant viruses. Preferred RNA plant virus vectors are positive strand single stranded RNA viruses. RNA plant virus vectors may be conveniently manipulated and introduced into cells in a DNA form instead of working directly with RNA vectors, in certain aspects. Viral vector derived from tobamoviruses are employed, in particular embodiments. Descriptions of suitable plant virus vectors that may be modified so as to contain an inhibitory RNA encoding region in functional combination with a promoter, as well as how to make and use such vectors, can be found at least in PCT publication number WO 93/03161, Kumagai et al., Proc. Natl. Acad. Sci. USA 90:427-430 (1993). Specific procedures and vectors previously used with wide success in plants are described, for example, by Bevan, Nucl. Acids Res. (1984) 12, 8711-8721), and Guerineau and Mullineaux, (1993) Plant transformation and expression vectors. In: Plant Molecular Biology Labfax (Croy RRD ed) Oxford, BIOS Scientific Publishers, pp 121-148.

Infectious RNAs from TTO1/PSY+, TTO1/PSY−, TTO1A/PDS+, TTO1/PDS− can be prepared by in vitro transcription using SP6 DNA-dependent RNA polymerase, and plant cells can be mechanically inoculated, in certain aspects (Dawson, et al., Adv. Virus Res. 38:307 (1990), which reference is incorporated by reference herein). The hybrid viruses spread throughout all the non-inoculated upper leaves as verified by transmission electron microscopy, local lesion infectivity assay, and polymerase chain reaction (PCR) amplification, for example. The viral symptoms comprise one or more of distortion of systemic leaves, plant stunting, and mild chlorosis, for example.

The invention described herein provides new methods for reducing the expression of BYDV MP genes, DNA constructs for practicing the methods, cells transformed by these genetic constructions, and higher organisms comprising the transformed cells. A vector that comprises the construct may be used in transformation of one or more plant cells to introduce the construct stably into the genome, so that it is stably inherited from one generation to the next. This is preferably followed by regeneration of a plant from such cells to produce a transgenic plant, in particular embodiments.

Thus, in further aspects, the present invention also provides the use of the construct or vector in production of a transgenic plant, methods of transformation of cells and plants, plant and microbial (particularly Agrobacterium) cells, and various plant products, for example. Suitable promoters include, for example, the Cauliflower Mosaic Virus 35S (CaMV 35S) gene promoter that is expressed at a high level in virtually all plant tissues (Benfey et al, 1990a and 1990b) and the maize glutathione-S-transferase isoform II (GST-II-27) gene promoter that is activated in response to application of exogenous safener (WO93/01294, ICI Ltd). The GST-II-27 gene promoter has been shown to be induced by certain chemical compounds which can be applied to growing plants. The promoter is functional in both monocotyledons and dicotyledons. It can therefore be used to control BYDV MP expression in a variety of genetically modified plants, including, for example, the exemplary field crops such as canola, sunflower, tobacco, sugarbeet, cotton; cereals such as wheat, barley, rice, maize, sorghum; fruit such as tomatoes, mangoes, peaches, apples, pears, strawberries, bananas, and melons; and vegetables such as carrot, lettuce, cabbage and onion. The GST-II-27 promoter is also suitable for use in a variety of tissues, including roots, leaves, stems and reproductive tissues.

Tobamoviruses, whose genomes consist of one plus-sense RNA strand of approximately 6.4 kb, replicate solely in the cytoplasm and can be used as episomal RNA vectors to alter plant biochemical pathways, in certain aspects of the invention. Hybrid tobacco mosaic (TMV)/odoritoglosum ringspot viruses (ORSV) have been used previously to express heterologous enzymes in transfected plants (Donson, et al., Proc. Natl. Acad. Sci. USA 88:7204 (1991) and Kumagai, et al., Proc. Natl. Acad. Sci. USA 90:427-430 (1993), minus-Sense RNA Strand (Miller, et al.). Infectious RNA transcripts from viral cDNA clones encode proteins involved in RNA replication, movement, and encapsidation.

Subgenomic RNA for messenger RNA synthesis is controlled by internal promoters located on the minus-sense RNA strand (N. benthamiana plants were inoculated with in vitro transcripts as described previously [W. O. Dawson, et al., Proc. Natl. Acad. Sci. USA 83:1832 (1986)]). Insertion of foreign genes into a specific location under the control of an additional subgenomic RNA promoter have resulted in systemic and stable expression of neomycin phosphotransferase and alpha-trichosanthin (Donson, et al., Proc. Natl. Acad. Sci. USA 88:7204 (1991) and Kumagai, et al., Proc. Natl. Acad. Sci. USA 90:427-430 (1993), which references are incorporated by reference as if set forth herein in their entirety.

There are numerous ways to produce the DNA constructs of the invention. Techniques for manipulating polynucleotides, e.g., restriction endonuclease digestion and ligation, are well known to a person of ordinary skill in the art. These conventional polynucleotide manipulation techniques may be used to produce and use the genetic construction of the invention. While some optimization of standard techniques may be employed to produce the subject genetic constructions, significant experimentation is not required.

The DNA constructs of the present invention comprise a promoter region in functional combination with an inhibitory RNA. The promoter region is selected so as to be capable of driving the transcription of a polynucleotide sequence in a host cell of interest. Thus, for example, when the eukaryotic cell is a plant cell, the promoter is selected so as to be able to drive transcription in plant cells. Promoters capable of functioning in a given eukaryotic cell are well known to a person of ordinary skill in the art. Examples of promoters capable of driving transcription in a cell of interest can be found at least in Goeddel et al., Gene Expression Technology Methods in Enzymology Volume 185, Academic Press, San Diego (1991), Ausubel et al., Protocols in Molecular Biology, Wiley Interscience (1994), and similar publications which references are incorporated by reference as if set forth herein in their entirety. When the cell for transformation is a plant cell, the RNA virus subgenomic promoters are used as promoter regions, in specific embodiments. RNA virus subgenomic promoters are described at least in Dawson and Lehto, Advances in Virus Research, 38:307-342, and PCT published application WO 93/03161, which references are incorporated by reference as if set forth herein in their entirety.

The promoter driving transcription of the inhibitory RNA encoding region of the subject DNA constructs may be selected so as have a level of transcriptional activity sufficient to achieve the desired degree of expression of the target gene inhibitory RNA of interest. The promoter may be native or heterologous to the cell for genetic modification.

The promoter may also be native or heterologous to the base vector, i.e., the portion of the vector other than the promoter and the inhibitory RNA encoding region. The promoter may be inducible or constitutive, in specific embodiments. In certain aspects, strong promoters are used to drive transcription of the inhibitory RNA encoding polynucleotide when the target RNA is highly expressed.

Promoters for fission yeast are well-known in the art. For example, adh1+ (constitutive high expression), fbp1+ (carbon source responsive), a tetracycline-repressible system based on the CaMV promoter, and the nmt1+ (no message in thiamine) promoter, which is the most frequently used, are commonly used promoters in fission yeast. There are three versions of nmt1+ promoter: the full strength promoter, and two attenuated versions that have reduced activity both in repressed and induced conditions (indicated below as nmt* and nmt**; see references). Several different polylinkers are available in the REP/RIP series of nmt vectors (see vector database). The concentration of thiamine can be adjusted for partial activation. Full induction: no thiamine. Full repression: 15 μM thiamine (5 μg/ml). Partial induction (described in this reference): 0.05 μM thiamine (0.016 μg/ml).

The nmt1 promoter does not switch off completely, and the ability to construct a “shutoff” plasmid depends very much on the protein being expressed and the sensitivity of the cell to dosage of that particular protein. Many genes expressed under nmt1 control are able to complement even in the presence of thiamine in the weakest promoter, but there are also numerous examples of genes that can be successfully shut off to generate a null phenotype. Thus, the utility of this promoter for plasmid shut-off experiments may be determined empirically for each gene. A comparison of promoter activity was published in Forsburg, (1993). Nucl. Acids Res. 21, 2955-2956, which is herein incorporated by reference in its entirety.

The invention also provides methods of inhibiting the activities of BYDV MP in a eukaryotic cell. As a consequence of providing the subject methods of reducing gene expression in eukaryotic cell, the subject invention also provides methods of producing a eukaryotic cell having reduced expression of a gene of interest and eukaryotic cells that have reduced expression of a gene of interest, as produced by the methods of the invention. Reduction of gene expression may be achieved by introducing one or more of the vectors of the invention into a eukaryotic cell. The vector used to transform the cell of interest comprises an inhibitory RNA encoding polynucleotide that encodes an inhibitory RNA specific for the BYDV MP gene, in specific aspects of the invention. The method of reducing expression of the gene may comprise the step of introducing the subject genetic vector into a host cell that is capable of expressing the gene of interest under certain environmental conditions. The vector may be introduced into a cell of interest by any of a variety of well known transformation methods. Such methods include, for example, infection, transfection, electroporation, ballistic projectile transformation, conjugation, and the like.

An additional optional feature of a construct used in accordance with the present invention is a transcriptional terminator. The transcriptional terminator from nopaline synthase gene of Agrobacterium tumefaciens (Depicker, A., et al (1982), J. Mol. Appl. Genet., 1: 561-573) may be used, and is experimentally exemplified below. Other suitable transcriptional terminators include but are not restricted to those from soybean actin, ribulose bisphosphate carboxylase of Nicotiana plumbaginifolia (Poulson, C., et al (1986), Mol. Gen. Genet., 205: 193-200) and alpha amylase of wheat (Baulcombe, D. C., et al (1987), Mol. Gen. Genet., 209: 33-40). A transcriptional terminator sequence foreign to the virus may not be included in a construct of the invention in particular when the viral sequences included in the construct include one or more transcriptional terminator sequences. Those skilled in the art are well able to construct vectors and design protocols for recombinant gene expression. For further details see, for example, Molecular Cloning: a Laboratory Manual: 2nd edition, Sambrook et al, 1989, Cold Spring Harbor Laboratory Press.

Many known techniques and protocols for manipulation of nucleic acid, for example in preparation of nucleic acid constructs, mutagenesis, sequencing, introduction of DNA into cells and gene expression, and analysis of proteins, are described in detail in Protocols in Molecular Biology, Second edition, Ausubel et al. eds., John Wiley & Sons, 1992. For introduction into a plant cell, the nucleic acid construct may be in the form of a recombinant vector, for example an Agrobacterium binary vector. Microbial host cells, such as bacterial and especially Agrobacterium host cells, comprising a construct according to the invention or a vector that includes such a construct, particularly a binary vector suitable for stable transformation of a plant cell, are also provided by the present invention.

Nucleic acid molecules, constructs and vectors according to the present invention may be provided isolated and/or purified (i.e. from their natural environment), in substantially pure or homogeneous form, or free or substantially free of other nucleic acid. Nucleic acid according to the present invention may be wholly or partially synthetic. The term “isolate” encompasses all these possibilities.

The construct or vector carrying inhibitory RNA to down-regulate BYDV MP or carrying a gene for a suppressor of BYDV MP identified using the methods of the invention, can be used to produce a transgenic plant, in certain aspects of the invention. The construct or vector is stably incorporated into a plant cell. Any appropriate method of plant transformation may be used to generate plant cells comprising a construct within the genome in accordance with the present invention. Following transformation, plants may be regenerated from transformed plant cells and tissue. Successfully transformed cells and/or plants, i.e. with the construct incorporated into their genome, may be selected following introduction of the nucleic acid into plant cells, optionally followed by regeneration into a plant, e.g. using one or more marker genes such as antibiotic resistance. Selectable genetic markers may be used comprising, for example, chimaeric genes that confer selectable phenotypes such as resistance to antibiotics, including kanamycin, hygromycin, phosphinotricin, chlorsulfuron, methotrexate, gentamycin, spectinomycin, imidazolinones and glyphosate, for example. When introducing a nucleic acid into a cell, certain considerations must be taken into account, well known to those skilled in the art. The nucleic acid to be inserted should be assembled within a construct that comprises effective regulatory elements that will drive transcription. There must be available a method of transporting the construct into the cell. Once the construct is within the cell membrane, integration into the endogenous chromosomal material occurs, in specific embodiments. Finally, as far as plants are concerned the target cell type are such that cells can be regenerated into whole plants, in specific aspects of the invention.

S. pombe plasmids comprise of one or more of a bacterial origin of replication and selectable marker (for example, an antibiotic resistance gene), a yeast selectable marker (for example, a metabolic marker) and an autonomous replication sequence (ars) that is responsible for high frequency of transformation, in particular aspects of the invention. There are no single copy centromere plasmids, as there are in budding yeast, because the fission yeast centromere is too big to be easily encompassed on a shuttle vector. Other features may include various promoters and fusion or tagging sequences.

Commonly used yet exemplary cloning markers for yeast include ade1; ade6; arg3; CAN1; his3; his7; leu1; LEU2 (LEU2 from S. cerevisiae complements leu1); sup3-5 (which is a nonsense suppressor that rescues ade6-704); ade6-704 (which provides for colonies that are dark red in limiting adenine and in the presence of the sup3-5 marker on a plasmid, which is slightly toxic in high copy; the colonies are pink due to plasmid loss and white if the sup3-5 marker integrates); ura4; and URA3.

Plants transformed with the DNA segment comprising the inhibitory RNA or suppressor may be produced by standard techniques that are already known for the genetic manipulation of plants. DNA can be transformed into plant cells using any suitable technology, such as a disarmed Ti-plasmid vector carried by Agrobacterium exploiting its natural gene transfer ability (EP-A-270355, EP-A-0116718, NAR 12(22) 8711-87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser—see attached) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d), for example. Physical methods for the transformation of plant cells are reviewed, for example, in Oard, 1991, Biotech. Adv. 9: 1-11.

Agrobacterium transformation is widely used by those skilled in the art to transform dicotyledonous species. Production of stable, fertile transgenic plants in almost all economically relevant monocot plants has been described (Toriyama, et al. (1988) Bio/Technology 6, 1072-1074; Zhang, et al. (1988) Plant Cell Rep. 7, 379-384; Zhang, et al. (1988) Theor Appl Genet 76, 835-840; Shimamoto, et al. (1989) Nature 338, 274-276; Datta, et al. (1990) Bio/Technology 8, 736-740; Christou, et al. (1991) Bio/Technology 9, 957-962; Peng, et al. (1991) International Rice Research Institute, Manila, Philippines 563-574; Cao, et al. (1992) Plant Cell Rep. 11, 585-591; Li, et al. (1993) Plant Cell Rep. 12, 250-255; Rathore, et al. (1993) Plant Molecular Biology 21, 871-884; Fromm, et al. (1990) Bio/Technology 8, 833-839; Gordon-Kamm, et al. (1990) Plant Cell 2, 603-618; D'Halluin, et al. (1992) Plant Cell 4, 1495-1505; Walters, et al. (1992) Plant Molecular Biology 18, 189-200; Koziel, et al. (1993) Biotechnology 11, 194-200; Vasil, I. K. (1994) Plant Molecular Biology 25, 925-937; Weeks, et al. (1993) Plant Physiology 102, 1077-1084; Somers, et al. (1992) Bio/Technology 10, 1589-1594; WO92/14828). In particular, Agrobacterium mediated transformation is now emerging also as a highly efficient transformation method in monocots (Hiei et al. (1994) The Plant Journal 6, 271-282).

The generation of fertile transgenic plants has been achieved in the cereals rice, maize, wheat, oat, and barley (reviewed in Shimamoto, K. (1994) Current Opinion in Biotechnology 5, 158-162; Vasil, et al. (1992) Bio/Technology 10, 667-674; Vain et al., 1995, Biotechnology Advances 13 (4): 653-671; Vasil, 1996, Nature Biotechnology 14 page 702). These references are incorporated by reference as if set forth herein in their entirety. Following transformation, a plant may be regenerated, e.g. from single cells, callus tissue, or leaf discs, for example, as is standard in the art. Almost any plant can be entirely regenerated from cells, tissues and organs of the plant. Available techniques are reviewed, for example, in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The particular choice of a transformation technology may be determined by its efficiency to transform certain plant species as well as the experience and preference of the person practicing the invention with a particular methodology of choice. It will be apparent to the skilled person that the particular choice of a transformation system to introduce nucleic acid into plant cells is not essential to or a limitation of the invention, nor is the choice of technique for plant regeneration. Also according to the invention there is provided a plant cell having incorporated into its genome a DNA construct as disclosed. A further aspect of the present invention provides a method of making such a plant cell involving introduction of a vector including the construct into a plant cell. Such introduction is followed by recombination between the vector and the plant cell genome to introduce the sequence of nucleotides into the genome, in particular aspects. RNA encoded by the introduced nucleic acid construct may then be transcribed in the cell and descendants thereof, including cells in plants regenerated from transformed material. A gene stably incorporated into the genome of a plant is passed from generation to generation to descendants of the plant, so such descendants show the desired phenotype, in specific embodiments.

As noted, in particular embodiments of the invention, transcription from the construct in the genome of a plant cell yields a replicating inhibitory RNA able to down-regulate expression of BYDV MP in the cell. Other and further aspects, features, and advantages of the present invention will be apparent from the following description of the presently preferred embodiments of the invention. These embodiments are given for the purpose of disclosure.

### VII. Plant Promoters

Promoters that are useful for plant transgene expression include those that are inducible, viral, synthetic, constitutive as described (Poszkowski et al., 1989; Odell et al., 1985), temporally regulated, spatially regulated, and spatio-temporally regulated (Chau et al., 1989).

A number of plant promoters have been described with various expression characteristics. Examples of some constitutive promoters which have been described include the rice actin 1 (Wang et al., 1992; U.S. Pat. No. 5,641,876), CaMV 35S (Odell et al., 1985), CaMV 19S (Lawton et al., 1987), nos (Ebert et al., 1987), Adh (Walker et al., 1987), and sucrose synthase (Yang & Russell, 1990).

Examples of tissue specific promoters that have been described include the lectin (Vodkin et al., 1983; Lindstrom et al., 1990), corn alcohol dehydrogenase 1 (Vogel et al., 1989; Dennis et al, 1984), corn light harvesting complex (Simpson, 1986; Bansal et al., 1992), corn heat shock protein (Odell et al., 1985; Rochester et al., 1986), pea small subunit RuBP carboxylase (Poulsen et al., 1986; Cashmore et al., 1983), Ti plasmid mannopine synthase (Langridge et al., 1989), Ti plasmid nopaline synthase (Langridge et al., 1989), petunia chalcone isomerase (Van Tunen et al., 1988), bean glycine rich protein 1 (Keller et al., 1989), truncated CaMV 35S (Odell et al., 1985), potato patatin (Wenzler et al., 1989), root cell (Conkling et al., 1990), maize zein (Reina et al., 1990; Kriz et al., 1987; Wandelt and Feix, 1989; Langridge and Feix, 1983; Reina et al., 1990), globulin-1 (Belanger and Kriz et al., 1991), □ tubulin, cab (Sullivan et al., 1989), PEPCase (Hudspeth & Grula, 1989), R gene complex-associated promoters (Chandler et al., 1989), and chalcone synthase promoters (Franken et al., 1991).

Inducible promoters that have been described include ABA- and turgor-inducible promoters, the promoter of the auxin-binding protein gene (Schwob et al., 1993), the UDP glucose flavonoid glycosyl-transferase gene promoter (Ralston et al., 1988); the MPI proteinase inhibitor promoter (Cordero et al., 1994), and the glyceraldehyde-3-phosphate dehydrogenase gene promoter (Kohler et al., 1995; Quigley et al., 1989; Martinez et al., 1989).

A class of genes that are expressed in an inducible manner are glycine-rich proteins GRPs). GRPs are a class of proteins characterized by their high content of glycine residues, which often occur in repetitive blocks (Goddemeier et al., 1998). Many GRPs are thought to be structural wall proteins or RNA-binding proteins (Mar Alba et al., 1994). Genes encoding glycine rich proteins have been described, for example, from maize (Didierjean et al., 1992; Baysdorfer, Genbank Accession No. AF034945) sorghum (Cretin and Puigdomenech, 1990), and rice (Lee et al., Genbank Accession No. AF009411).

### VIII. Plant Transformation Constructs

The construction of vectors that may be employed in conjunction with plant transformation techniques according to the invention will be known to those of skill of the art in light of the present disclosure (see for example, Sambrook et al., 1989; Gelvin et al. 1990). In one embodiment, sequences of the invention are employed for directing the expression of a selected coding region that encodes a particular protein or polypeptide product, although in alternative embodiments the selected coding regions also may produce RNAs or DNAs that do not encode a gene product, e.g., antisense RNA or cosuppressor RNA. The inventors also contemplate that, where both a gene that is not necessarily a marker gene is employed in combination with a marker gene, one may employ the separate genes on either the same or different DNA segments for transformation. In the latter case, the different vectors are delivered concurrently to recipient cells to maximize cotransformation.

The choice of the particular selected coding regions used in accordance with the transformation of recipient cells will often depend on the purpose of the transformation. One of the major purposes of transformation of crop plants is to add commercially desirable, agronomically important traits to the plant. Such traits include, but are not limited to, resistance to barley yellow dwarf virus. In additional embodiments, transgenic plants of the invention also comprise herbicide resistance or tolerance; insect resistance or tolerance; disease resistance or tolerance (viral, bacterial, fungal, nematode); stress tolerance and/or resistance, as exemplified by resistance or tolerance to drought, heat, chilling, freezing, excessive moisture, salt stress, or oxidative stress; increased yields; food content and makeup; physical appearance; male sterility; drydown; standabilily; prolificacy; starch properties; oil quantity and quality, and the like.

In certain embodiments, the present inventors contemplate the transformation of a recipient cell with more than a transformation construct. Two or more transgenes can be created in a single transformation event using either distinct selected-protein encoding vectors, or using a single vector incorporating two or more gene coding sequences. Of course, any two or more transgenes of any description, such as those conferring, for example, downregulation of BYDV MP, herbicide, insect, disease (viral, bacterial, fungal, nematode) or drought resistance, male sterility, drydown, standability, prolificacy, starch properties, oil quantity and quality, or those increasing yield or nutritional quality may be employed as desired.

In other embodiments of the invention, it is contemplated that one may wish to employ replication-competent viral vectors for plant transformation. Such vectors include, for example, wheat dwarf virus (WDV) “shuttle” vectors, such as pW1-11 and PW1-GUS (Ugaki et al., 1991). These vectors are capable of autonomous replication in maize cells as well as E. coli, and as such may provide increased sensitivity for detecting DNA delivered to transgenic cells. A replicating vector also may be useful for delivery of genes flanked by DNA sequences from transposable elements such as Ac, Ds, or Mu. It has been proposed that transposition of these elements within the maize genome requires DNA replication (Laufs et al., 1990). It also is contemplated that transposable elements would be useful for introducing DNA fragments lacking elements necessary for selection and maintenance of the plasmid vector in bacteria, e.g., antibiotic resistance genes and origins of DNA replication. It also is proposed that use of a transposable element such as Ac, Ds, or Mu would actively promote integration of the desired DNA and hence increase the frequency of stably transformed cells.

It further is contemplated that one may wish to co-transform plants or plant cells with 2 or more vectors. Co-transformation may be achieved using a vector containing the marker and another gene or genes of interest. Alternatively, different vectors, e.g., plasmids, may contain the different genes of interest, and the plasmids may be concurrently delivered to the recipient cells. Using this method, the assumption is made that a certain percentage of cells in which the marker has been introduced, also have received the other gene(s) of interest. Thus, not all cells selected by means of the marker, will express the other proteins of interest which had been presented to the cells concurrently.

Vectors used for plant transformation may include, for example, plasmids, cosmids, YACs (yeast artificial chromosomes), BACs (bacterial artificial chromosomes) or any other suitable cloning system. It is contemplated that utilization of cloning systems with large insert capacities will allow introduction of large DNA sequences comprising more than one selected gene. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al. (1996).

Particularly useful for transformation are expression cassettes which have been isolated from such vectors. DNA segments used for transforming plant cells will, of course, generally comprise the cDNA, gene or genes which one desires to introduced into and have expressed in the host cells. These DNA segments can further include, in addition to a ZMGRP promoter, structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with vectors used in the current invention are as follows.

A. Regulatory Elements

Constructs prepared in accordance with the current invention will include an ZMGRP promoter or a derivative thereof. However, these sequences may be used in the preparation of transformation constructs which comprise a wide variety of other elements. One such application in accordance with the instant invention will be the preparation of transformation constructs comprising the ZMGRP promoter operably linked to a selected coding region. By including an enhancer sequence with such constructs, the expression of the selected protein may be enhanced. These enhancers often are found 5′ to the start of transcription in a promoter that functions in eukaryotic cells, but can often be inserted in the forward or reverse orientation 5′ or 3′ to the coding sequence. In some instances, these 5′ enhancing elements are introns. Deemed to be particularly useful as enhancers are the 5′ introns of the rice actin 1 and rice actin 2 genes. Examples of other enhancers which could be used in accordance with the invention include elements from the CaMV 35S promoter, octopine synthase genes (Ellis et al., 1987), the maize alcohol dehydrogenase gene, the maize shrunken 1 gene and promoters from non-plant eukaryotes (e.g., yeast; Ma et al., 1988).

Where an enhancer is used in conjunction with a ZMGRP promoter for the expression of a selected protein, it is believed that it will be preferred to place the enhancer between the promoter and the start codon of the selected coding region. However, one also could use a different arrangement of the enhancer relative to other sequences and still realize the beneficial properties conferred by the enhancer. For example, the enhancer could be placed 5′ of the promoter region, within the promoter region, within the coding sequence (including within any other intron sequences which may be present), or 3′ of the coding region.

In addition to introns with enhancing activity, other types of elements can influence gene expression. For example, untranslated leader sequences have been made to predict optimum or sub-optimum sequences and generate “consensus” and preferred leader sequences (Joshi, 1987). Preferred leader sequences are contemplated to include those which have sequences predicted to direct optimum expression of the attached coding region, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly, expressed in plants, and in maize in particular, will be most preferred.

Specifically contemplated for use in accordance with the present invention are vectors which include the ocs enhancer element. This element was first identified as a 16 bp palindromic enhancer from the octopine synthase (ocs) gene of Agrobacterium (Ellis et al., 1987), and is present in at least 10 other promoters (Bouchez et al., 1989). It is proposed that the use of an enhancer element, such as the ocs element and particularly multiple copies of the element, may be used to increase the level of transcription from adjacent promoters when applied in the context of monocot transformation.

Ultimately, the most desirable DNA segments for introduction into a plant genome may be homologous genes or gene families which encode a desired trait, and which are introduced under the control of the maize GRP promoter. The tissue-specific expression profile of the ZMGRP promoter will be of particular benefit in the expression of transgenes in plants. For example, it is envisioned that a particular use of the present invention may be the production of transformants comprising a transgene which is expressed in a tissue-specific manner, whereby the expression is enhanced by an actin 1 or actin 2 intron. For example, insect resistant protein may be expressed specifically in the roots which are targets for a number of pests including nematodes and the corn root worn.

It also is contemplated that expression of one or more transgenes may be obtained in all tissues but roots by introducing a constitutively expressed gene (all tissues) in combination with an antisense gene that is expressed only by the ZMGRP promoter. Therefore, expression of an antisense transcript encoded by the constitutive promoter would prevent accumulation of the respective protein encoded by the sense transcript. Similarly, antisense technology could be used to achieve temporally-specific or inducible expression of a transgene encoded by a ZMGRP promoter.

It also is contemplated that it may be useful to target DNA within a cell. For example, it may be useful to target introduced DNA to the nucleus as this may increase the frequency of transformation. Within the nucleus itself, it would be useful to target a gene in order to achieve site specific integration. For example, it would be useful to have a gene introduced through transformation replace an existing gene in the cell.

B. Terminators

Transformation constructs prepared in accordance with the invention will typically include a 3′ end DNA sequence that acts as a signal to terminate transcription and allow for the poly-adenylation of the mRNA produced by coding sequences operably linked to the maize GRP promoter. One type of terminator which may be used is a terminator from a gene encoding the small subunit of a ribulose-1,5-bisphosphate carboxylase-oxygenase (rbcS), and more specifically, from a rice rbcS gene. Where a 3′ end other than an rbcS terminator is used in accordance with the invention, the most preferred 3′ ends are contemplated to be those from the nopaline synthase gene of Agrobacterium tumefaciens (nos 3′ end) (Bevan et al, 1983), the terminator for the T7 transcript from the octopine synthase gene of Agrobacterium tumefaciens, and the 3′ end of the protease inhibitor I or II genes from potato or tomato. Regulatory elements such as Adh intron (Callis et al., 1987), sucrose synthase intron (Vasil et al., 1989) or TMV omega element (Gallie, et al., 1989), may further be included where desired. Alternatively, one also could use a gamma coixin, oleosin 3 or other terminator from the genus Coix.

C. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

A particular example of such a use concerns the direction of a protein conferring herbicide resistance, such as a mutant EPSPS protein, to a particular organelle such as the chloroplast rather than to the cytoplasm. This is exemplified by the use of the rbcS transit peptide, the chloroplast transit peptide described in U.S. Pat. No. 5,728,925, or the optimized transit peptide described in U.S. Pat. No. 5,510,471, which confers plastid-specific targeting of proteins. In addition, it may be desirable to target certain genes responsible for male sterility to the mitochondria, or to target certain genes for resistance to phytopathogenic organisms to the extracellular spaces, or to target proteins to the vacuole. A further use concerns the direction of enzymes involved in amino acid biosynthesis or oil synthesis to the plastid. Such enzymes include dihydrodipicolinic acid synthase which may contribute to increasing lysine content of a feed.

D. Marker Genes

One application of the maize GRP promoter of the current invention will be in the expression of marker proteins. By employing a selectable or screenable marker gene as, or in addition to, the gene of interest, one can provide or enhance the ability to identify transformants. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker genes are known to the art and can be employed in the practice of the invention.

Included within the terms selectable or screenable marker genes also are genes which encode a “secretable marker” whose secretion can be detected as a means of identifying or selecting for transformed cells. Examples include marker genes which encode a secretable antigen that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR S).

With regard to selectable secretable markers, the use of a gene that encodes a protein that becomes sequestered in the cell wall, and which protein includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

One example of a protein suitable for modification in this manner is extensin, or hydroxyproline rich glycoprotein (HPRG). The use of maize HPRG (Steifel et al., 1990) is preferred, as this molecule is well characterized in terms of molecular biology, expression and protein structure. However, any one of a variety of extensins and/or glycine-rich wall proteins (Keller et al., 1989) could be modified by the addition of an antigenic site to create a screenable marker.

One exemplary embodiment of a secretable screenable marker concerns the use of a HPRG sequence modified to include a 15 residue epitope from the pro-region of murine interleukin-1-β (IL-1-β). However, virtually any detectable epitope may be employed in such embodiments, as selected from the extremely wide variety of antigen:antibody combinations known to those of skill in the art. The unique extracellular epitope, whether derived from IL-1β or any other protein or epitopic substance, can then be straightforwardly detected using antibody labeling in conjunction with chromogenic or fluorescent adjuncts.

1. Selectable Markers

Many selectable marker coding regions may be used in connection with the ZMGRP promoter of the present invention including, but not limited to, neo (Potrykus et al., 1985) which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154, 204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan. Where a mutant EPSP synthase is employed, additional benefit may be realized through the incorporation of a suitable chloroplast transit peptide, CTP (U.S. Pat. No. 5,188,642) or OTP (U.S. Pat. No. 5,633,448) and use of a modified maize EPSPS (PCT Application WO 97/04103).

An illustrative embodiment of selectable markers capable of being used in systems to select transformants are the enzyme phosphinothricin acetyltransferase, such as bar from Streptomyces hygroscopicus or pat from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

Where one desires to employ bialaphos resistance in the practice of the invention, the inventor has discovered that particularly useful genes for this purpose are the bar or pat genes obtainable from species of Streptomyces (e.g., ATCC No. 21,705). The cloning of the bar gene has been described (Murakami et al., 1986; Thompson et al., 1987) as has the use of the bar gene in the context of plants (De Block et al., 1987; De Block et al., 1989; U.S. Pat. No. 5,550,318).

2. Screenable Markers

Screenable markers that may be employed include a □ glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalospbrin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; an aequorin gene (Prasher et al., 1985) which may be employed in calcium-sensitive bioluminescence detection; or a gene encoding for green fluorescent protein (Sheen et al., 1995; Haseloff et al., 1997; Reichel et al., 1996; Tian et al., 1997; WO 97/41228).

Genes from the maize R gene complex are contemplated to be particularly useful as screenable markers. The R gene complex in maize encodes a protein that acts to regulate the production of anthocyanin pigments in most seed and plant tissue. Maize strains can have one, or as many as four, R alleles which combine to regulate pigmentation in a developmental and tissue specific manner. Thus, an R gene introduced into such cells will cause the expression of a red pigment and, if stably incorporated, can be visually scored as a red sector. If a maize line carries dominant alleles for genes encoding for the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 which contains the rg-Stadler allele and TR112, a K55 derivative which is r-g, b, P1. Alternatively, any genotype of maize can be utilized if the C1 and R alleles are introduced together.

It further is proposed that R gene regulatory regions may be employed in chimeric constructs in order to provide mechanisms for controlling the expression of chimeric genes. More diversity of phenotypic expression is known at the R locus than at any other locus (Coe et al., 1988). It is contemplated that regulatory regions obtained from regions 5′ to the structural R gene would be valuable in directing the expression of genes for, e.g., insect resistance, herbicide tolerance or other protein coding regions. For the purposes of the present invention, it is believed that any of the various R gene family members may be successfully employed (e.g., P, S, Lc, etc.). However, the most preferred will generally be Sn (particularly Sn:bo13). Sn is a dominant member of the R gene complex and is functionally similar to the R and B loci in that Sn controls the tissue specific deposition of anthocyanin pigments in certain seedling and plant cells, therefore, its phenotype is similar to R.

Another screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for populational screening for bioluminescence, such as on tissue culture plates, or even for whole plant screening. The gene which encodes green fluorescent protein (GFP) is contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell or plant as fluorescence following illumination by particular wavelengths of light. Where use of a screenable marker gene such as lux or GFP is desired, the inventors contemplated that benefit may be realized by creating a gene fusion between the screenable marker gene and a selectable marker gene, for example, a GFP-NPTII gene fusion. This could allow, for example, selection of transformed cells followed by screening of transgenic plants or seeds.

### IX. Nucleic Acid-Based Expression Systems

1. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operativelly positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the beta-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the website of the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
Inducible Elements

| Element | Inducer | References |
|---|---|---|
| MT II | Phorbol Ester | Palmiter et al., 1982; |
| | (TFA) Heavy | Haslinger et al., 1985; |
| | metals | Searle et al., 1985; |
| | | Stuart et al., 1985; |
| | | Imagawa et al., 1987, |
| | | Karin et al., 1987; Angel |
| | | et al., 1987b; McNeall et |
| | | al., 1989 |
| MMTV (mouse | Glucocorticoids | Huang et al., 1981; Lee |
| mammary | | et al., 1981; Majors et |
| tumor virus) | | al., 1983; Chandler et |
| | | al., 1983; Lee et al., |
| | | 1984; Ponta et al., 1985; |
| | | Sakai et al., 1988 |
| β-Interferon | Poly(rI)x | Tavernier et al., 1983 |
| | Poly(rc) | |
| Adenovirus | E1A | Imperiale et al., 1984 |
| 5 E2 | | |
| Collagenase | Phorbol Ester | Angel et al., 1987a |
| | (TPA) | |
| Stromelysin | Phorbol Ester | Angel et al., 1987b |
| | (TPA) | |
| SV40 | Phorbol Ester | Angel et al., 1987b |
| | (TPA) | |
| Murine MX Gene | Interferon, | Hug et al., 1988 |
| | Newcastle | |
| | Disease Virus | |
| GRP78 Gene | A23187 | Resendez et al., 1988 |
| α-2- | IL-6 | Kunz et al., 1989 |
| Macroglobulin | | |
| Vimentin | Serum | Rittling et al., 1989 |
| MHC Class 1 | Interferon | Blanar et al., 1989 |
| Gene H-2κb | | |
| HSP70 | E1A, SV40 Large | Taylor et al., 1989, 1990a, |
| | T Antigen | 1990b |
| Proliferin | Phorbol Ester-TPA | Mordacq et al., 1989 |
| Tumor Necrosis | PMA | Hensel et al., 1989 |
| Factor α | | |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Non-limiting examples of such regions include: the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryote, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

i. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEMTM 11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with □ galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

j. Viral Vectors

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the [INVENTION]vaccines of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as delivery vectors in organisms due to their ability to integrate their genes into the host genome, transferring a large amount of foreign genetic material, infecting a broad spectrum of species and cell types and of being packaged in special cell lines (Miller, 1992).

In order to construct a retroviral vector of the invention, a nucleic acid (e.g., one encoding the molecule of interest) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al, 1989). Using antibodies against major histocompatibility complex class I and class II antigens, they demonstrated the infection of a variety of human cells that bore those surface antigens with an ecotropic virus in vitro (Roux et al., 1989).

2. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,931,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985), and any combination of such methods. Through the application of techniques such as these, organelle(s), cell(s), tissue(s) or organism(s) may be stably or transiently transformed.

a. Ex Vivo Transformation

Methods for transfecting vascular cells and tissues removed from an organism in an ex vivo setting are known to those of skill in the art. For example, cannine endothelial cells have been genetically altered by retrovial gene transfer in vitro and transplanted into a canine (Wilson et al., 1989). In another example, yucatan minipig endothelial cells were transfected by retrovirus in vitro and transplated into an artery using a double-ballonw catheter (Nabel et al., 1989). Thus, it is contemplated that cells or tissues may be removed and transfected ex vivo using the nucleic acids of the present invention. In particular aspects, the transplanted cells or tissues may be placed into an organism. In preferred facets, a nucleic acid is expressed in the transplated cells or tissues.

b. Injection

In certain embodiments, a nucleic acid may be delivered to an organelle, a cell, a tissue or an organism via one or more injections (i.e., a needle injection), such as, for example, subcutaneously, intradermally, intramuscularly, intravenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985). The amount of compound used may vary upon the nature of the compound as well as the organelle, cell, tissue or organism used.

c. Electroporation

In certain embodiments of the present invention, a nucleic acid is introduced into an organelle, a cell, a tissue or an organism via electroporation. Electroporation involves the exposure of a suspension of cells and DNA to a high voltage electric discharge. In some variants of this method, certain cell wall degrading enzymes, such as pectin degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre B lymphocytes have been transfected with human kappa immunoglobulin genes (Potter et al, 1984), and rat hepatocytes have been transfected with the chloramphenicol acetyltransferase gene (Tur Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon derived protoplasts is described by Dhir and Widholm in International Patent Application No. WO 9217598, incorporated herein by reference. Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

d. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV 1, BHK, NIH3T3 and HeLa cells were transfected with a neomycin marker gene (Chen and Okayama, 1987), and rat hepatocytes were transfected with a variety of marker genes (Rippe et al., 1990).

e. DEAE Dextran

In another embodiment, a nucleic acid is delivered into a cell using DEAE dextran-followed by polyethylene glycol. In this manner, reporter plasmids were introduced into mouse myeloma and erythroleukemia cells (Gopal, 1985).

f. Sonication Loading

Additional embodiments of the present invention include the introduction of a nucleic acid by direct sonic loading. LTK fibroblasts have been transfected with the thymidine kinase gene by sonication loading (Fechheimer et al., 1987).

g. Liposome Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome mediated nucleic acid delivery and expression of foreign DNA in vitro has been very successful (Nicolau and Sene, 1982; Fraley et al., 1979; Nicolau et al., 1987). The feasibility of liposome mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome encapsulated DNA (Kaneda et al., 1989). In other embodiments, a liposome may be complexed or employed in conjunction with nuclear non histone chromosomal proteins (HMG 1) (Kato et al., 1991). In yet further embodiments, a liposome may be complexed or employed in conjunction with both HVJ and HMG 1. In other embodiments, a delivery vehicle may comprise a ligand and a liposome.

h. Receptor Mediated Transfection

Still further, a nucleic acid may be delivered to a target cell via receptor mediated delivery vehicles. These take advantage of the selective uptake of macromolecules by receptor mediated endocytosis that will be occurring in a target cell. In view of the cell type specific distribution of various receptors, this delivery method adds another degree of specificity to the present invention.

Certain receptor mediated gene targeting vehicles comprise a cell receptor specific ligand and a nucleic acid binding agent. Others comprise a cell receptor specific ligand to which the nucleic acid to be delivered has been operatively attached. Several ligands have been used for receptor mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell specific binding. For example, lactosyl ceramide, a galactose terminal asialganglioside, have been incorporated into liposomes and observed an increase in the uptake of the insulin gene by hepatocytes (Nicolau et al., 1987). It is contemplated that the tissue specific transforming constructs of the present invention can be specifically delivered into a target cell in a similar manner.

i. Microprojectile Bombardment

Microprojectile bombardment techniques can be used to introduce a nucleic acid into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA coated microprojectiles to a high velocity allowing them to pierce cell membranes and enter cells without killing them (Klein et al., 1987). There are a wide variety of microprojectile bombardment techniques known in the art, many of which are applicable to the invention.

Microprojectile bombardment may be used to transform various cell(s), tissue(s) or organism(s), such as for example any plant species. Examples of species which have been transformed by microprojectile bombardment include monocot species such as maize (PCT Application WO 95106128), barley (Ritala et al., 1994; Hensgens et al., 1993), wheat (U.S. Pat. No. 5,563,055, incorporated herein by reference), rice (Hensgens et al., 1993), oat (Torbet et al., 1995; Torbet et al., 1998), rye (Hensgens et al., 1993), sugarcane (Bower et al., 1992), and sorghum (Casas et al., 1993; Hagio et al., 1991); as well as a number of dicots including tobacco (Tomes et al., 1990; Buising and Benbow, 1994), soybean (U.S. Pat. No. 5,322,783, incorporated herein by reference), sunflower (Knittel et al. 1994), peanut (Singsit et al., 1997), cotton (McCabe and Martinell, 1993), tomato (VanEck et al. 1995), and legumes in general (U.S. Pat. No. 5,563,055, incorporated herein by reference).

In this microprojectile bombardment, one or more particles may be coated with at least one nucleic acid and delivered into cells by a propelling force. Several devices for accelerating small particles have been developed. One such device relies on a high voltage discharge to generate, an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

3. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co expressed with other selected RNAs or proteinaceous sequences in the same host cell. Co expression may be achieved by co transfecting the host cell with two or more distinct recombinant vectors. Alternatively, a single recombinant vector may be constructed to include multiple distinct coding regions for RNAs, which could then be expressed in host cells transfected with the single vector.

A tissue may comprise a host cell or cells to be transformed with a [INVENTION]. The tissue may be part or separated from an organism. In certain embodiments, a tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stern cells, stomach, testes, anthers, ascite tissue, cobs, ears, flowers, husks, kernels, leaves, meristematic cells, pollen, root tips, roots, silk, stalks, and all cancers thereof.

In certain embodiments, the host cell or tissue may be comprised in at least one organism. In certain embodiments, the organism may be, but is not limited to, a prokayote (e.g., a eubacteria, an archaea) or an eukaryote, as would be understood by one of ordinary skill in the art (see, for example, webpage http://phylogeny.arizona.edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F, lambda, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK® Gold Cells (STRATAGENE®), La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

4. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN®) and BACPACK™ BACULOVIRUS EXPRESSION SYSTEM FROM CLONTECH®.

Other examples of expression systems include STRATAGENE®'s COMPLETE CONTROL® Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed”, i.e., expressed in increased levels relative to its natural expression in cells. Such overexpression may be assessed by a variety of methods, including radio labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g. 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

### X. Transformation Techniques Directed to Plants

Exemplary transformation techniques for plants are described.

1. Agrobacterium Mediated Transformation

Agrobacterium mediated transfer is a widely applicable system for introducing nucleic acid(s) into a plant cell because the nucleic acid (i.e., DNA) can be introduced into a whole plant tissue, thereby bypassing the need for regeneration of an intact plant from a protoplast. The use of Agrobacterium mediated plant integrating vectors to introduce DNA into plant cells is well known in the art (see, for example, Fraley et al., 1985; Rogers et al., 1987; and U.S. Pat. No. 5,563,055, incorporated herein by reference).

Agrobacterium mediated transformation is most efficient in a dicotyledonous plant and is the preferable method for transformation of a dicot, including Arabidopsis, tobacco, tomato, and potato. Indeed, while Agrobacterium mediated transformation has been routinely used with dicotyledonous plants for a number of years, it has only recently become applicable to monocotyledonous plants. Advances in Agrobacterium mediated transformation techniques have now made the technique applicable to nearly all monocotyledonous plants. For example, Agrobacterium mediated transformation techniques have now been applied to rice (Hiei et al., 1997; Zhang et al., 1997; U.S. Pat. No. 5,591,616, incorporated herein by reference), wheat (McCormac et al., 1998), barley (Tingay et al, 1997; McCormac et al., 1998), and maize (Ishidia et al., 1996).

Modern Agrobacterium transformation vectors are capable of replication in E. coli as well as Agrobacterium, allowing for convenient manipulations (Klee et al., 1985). Moreover, recent technological advances in vectors for Agrobacterium mediated gene transfer have improved the arrangement of genes and restriction sites in the vectors to facilitate the construction of vectors capable of expressing various peptides, polypeptide or protein coding nucleic acids. The vectors described (Rogers et al., 1987) have convenient multi linker regions flanked by a promoter and a polyadenylation site for direct expression of inserted coding region and are suitable for present purposes. In addition, Agrobacterium containing both armed and disarmed Ti genes can be used for the transformations. In those plant strains where Agrobacterium mediated transformation is efficient, it is the method of choice because of the facile and defined nature of the gene transfer.

2. Other Plant Transformation Methods

Transformation of a plant protoplast can be achieved using methods based on calcium phosphate precipitation, polyethylene glycol treatment, electroporation, and combinations of these treatments (see, e.g., Potrykus et al., 1985; Lorz et al., 1985; Omirulleh et al., 1993; Fromm et al., 1986; Uchimiya et al., 1986; Callis et al., 1987; Marcotte et al., 1988).

Application of these systems to different plant strains depends upon the ability to regenerate that particular plant strain from a protoplast. Illustrative methods for the regeneration of cereals from protoplasts have been described (Fujimara et al., 1985; Toriyama et al., 1986; Yamada et al., 1986; Abdullah et al., 1986; Omirulleb et al., 1993 and U.S. Pat. No. 5,508,184; each incorporated herein by reference). Examples of the use of direct uptake transformation of cereal protoplasts include transformation of rice (Ghosh Biswas et al., 1994), sorghum (Battraw and Hall, 1991), barley (Lazerri, 1995), oat (Zheng and Edwards, 1990) and maize (Omirulleh et al., 1993).

To transform a plant strain that cannot be successfully regenerated from a protoplast, other ways to introduce DNA into intact cells or tissues can be utilized. For example, regeneration of cereals from immature embryos or explants can be effected as described (Vasil, 1989). Also, silicon carbide fiber mediated transformation may be used with or without protoplasting (Kaeppler, 1990; Kaeppler et al., 1992; U.S. Pat. No. 5,563,055, incorporated herein by reference). Transformation with this technique is accomplished by agitating a silicon carbide fiber together with a cell in a DNA solution. DNA passively enters as the cell(s) are punctured. This technique has been used successfully with, for example, the monocot cereals maize (Thompson, 1995; PCT Application WO 95/06128, incorporated herein by reference) and rice (Nagatani, 1997).

### XI. Site-Specific Integration and Excision of Transgenes

It is specifically contemplated by the inventors that one could employ techniques for the site-specific integration or excision of transformation constructs prepared in accordance with the instant invention. An advantage of site-specific integration or excision is that it can be used to overcome problems associated with conventional transformation techniques, in which transformation constructs typically randomly integrate into a host genome in multiple copies. This random insertion of introduced DNA into the genome of host cells can be lethal if the foreign DNA inserts into an essential gene. In addition, the expression of a transgene may be influenced by “position effects” caused by the surrounding genomic DNA. Further, because of difficulties associated with plants possessing multiple transgene copies, including gene silencing, recombination and unpredictable inheritance, it is typically desirable to control the copy number of the inserted DNA, often only desiring the insertion of a single copy of the DNA sequence.

Site-specific integration or excision of transgenes or parts of transgenes can be achieved in plants by means of homologous recombination (see, for example, U.S. Pat. No. 5,527,695, specifically incorporated herein by reference in its entirety). Homologous recombination is a reaction between any pair of DNA sequences having a similar sequence of nucleotides, where the two sequences interact (recombine) to form a new recombinant DNA species. The frequency of homologous recombination increases as the length of the shared nucleotide DNA sequences increases, and is higher with linearized plasmid molecules than with circularized plasmid molecules. Homologous recombination can occur between two DNA sequences that are less than identical, but the recombination frequency declines as the divergence between the two sequences increases.

Introduced DNA sequences can be targeted via homologous recombination by linking a DNA molecule of interest to sequences sharing homology with endogenous sequences of the host cell. Once the DNA enters the cell, the two homologous sequences can interact to insert the introduced DNA at the site where the homologous genomic DNA sequences were located. Therefore, the choice of homologous sequences contained on the introduced DNA will determine the site where the introduced DNA is integrated via homologous recombination. For example, if the DNA sequence of interest is linked to DNA sequences sharing homology to a single copy gene of a host plant cell, the DNA sequence of interest will be inserted via homologous recombination at only that single specific site. However, if the DNA sequence of interest is linked to DNA sequences sharing homology to a multicopy gene of the host eukaryotic cell, then the DNA sequence of interest can be inserted via homologous recombination at each of the specific sites where a copy of the gene is located.

DNA can be inserted into the host genome by a homologous recombination reaction involving either a single reciprocal recombination (resulting in the insertion of the entire length of the introduced DNA) or through a double reciprocal recombination (resulting in the insertion of only the DNA located between the two recombination events). For example, if one wishes to insert a foreign gene into the genomic site where a selected gene is located, the introduced DNA should contain sequences homologous to the selected gene. A single homologous recombination event would then result in the entire introduced DNA sequence being inserted into the selected gene. Alternatively, a double recombination event can be achieved by flanking each end of the DNA sequence of interest (the sequence intended to be inserted into the genome) with DNA sequences homologous to the selected gene. A homologous recombination event involving each of the homologous flanking regions will result in the insertion of the foreign DNA. Thus only those DNA sequences located between the two regions sharing genomic homology become integrated into the genome.

Although introduced sequences can be targeted for insertion into a specific genomic site via homologous recombination, in higher eukaryotes homologous recombination is a relatively rare event compared to random insertion events. In plant cells, foreign DNA molecules find homologous sequences in the cell's genome and recombine at a frequency of approximately 0.5-4.2×10⁻⁴. Thus any transformed cell that contains an introduced DNA sequence integrated via homologous recombination will also likely contain numerous copies of randomly integrated introduced DNA sequences. Therefore, to maintain control over the copy number and the location of the inserted DNA, these randomly inserted DNA sequences can be removed. One manner of removing these random insertions is to utilize a site-specific recombinase system. In general, a site specific recombinase system consists of three elements: two pairs of DNA sequence (the site—specific recombination sequences) and a specific enzyme (the site-specific recombinase). The site-specific recombinase will catalyze a recombination reaction only between two site-specific recombination sequences.

A number of different site specific recombinase systems could be employed in accordance with the instant invention, including, but not limited to, the Cre/lox system of bacteriophage P1 (U.S. Pat. No. 5,658,772, specifically incorporated herein by reference in its entirety), the FLP/FRT system of yeast (Golic and Lindquist, 1989), the Gin recombinase of phage Mu (Maeser and Kahmann, 1991), the Pin recombinase of E. coli (Enomoto et al., 1983), and the R/RS system of the pSR1 plasmid (Araki et al., 1992). The bacteriophage PI Cre/lox and the yeast FLP/FRT systems constitute two particularly useful systems for site specific integration or excision of transgenes. In these systems, a recombinase (Cre or FLP) will interact specifically with its respective site-specific recombination sequence (lox or FRT; respectively) to invert or excise the intervening sequences. The sequence for each of these two systems is relatively short (34 bp for lox and 47 bp for FRT) and therefore, convenient for use with transformation vectors.

The FLP/FRT recombinase system has been demonstrated to function efficiently in plant cells. Experiments on the performance of the FLP/FRT system in both maize and rice protoplasts indicate that FRT site structure, and amount of the FLP protein present, affects excision activity. In general, short incomplete FRT sites leads to higher accumulation of excision products than the complete full-length FRT sites. The systems can catalyze both intra- and intermolecular reactions in maize protoplasts, indicating its utility for DNA excision as well as integration reactions. The recombination reaction is reversible and this reversibility can compromise the efficiency of the reaction in each direction. Altering the structure of the site-specific recombination sequences is one approach to remedying this situation. The site-specific recombination sequence can be mutated in a manner that the product of the recombination reaction is no longer recognized as a substrate for the reverse reaction, thereby stabilizing the integration or excision event.

In the Cre-lox system, discovered in bacteriophage P1, recombination between loxP sites occurs in the presence of the Cre recombinase (see, e.g., U.S. Pat. No. 5,658,772, specifically incorporated herein by reference in its entirety). This system has been utilized to excise a gene located between two lox sites which had been introduced into a yeast genome (Sauer, 1987). Cre was expressed from an inducible yeast GAL1 promoter and this Cre gene was located on an autonomously replicating yeast vector.

Since the lox site is an asymmetrical nucleotide sequence, lox sites on the same DNA molecule can have the same or opposite orientation with respect to each other. Recombination between lox sites in the same orientation results in a deletion of the DNA Segment located between the two lox sites and a connection between the resulting ends of the original DNA molecule. The deleted DNA segment forms a circular molecule of DNA. The original DNA molecule and the resulting circular molecule each contain a single lox site. Recombination between lox sites in opposite orientations on the same DNA molecule result in an inversion of the nucleotide sequence of the DNA segment located between the two lox sites. In addition, reciprocal exchange of DNA segments proximate to lox sites located on two different DNA molecules can occur. All of these recombination events are catalyzed by the product of the Cre coding region.

### XII. Production and Characterization of Stably Transformed Plants

After effecting delivery of exogenous DNA to recipient cells, the next steps generally concern identifying the transformed cells for further culturing and plant regeneration. As mentioned herein, in order to improve the ability to identify transformants, one may desire to employ a selectable or screenable marker gene as, or in addition to, the expressible gene of interest. In this case, one would then generally assay the potentially transformed cell population by exposing the cells to a selective agent or agents, or one would screen the cells for the desired marker gene trait.

A. Selection

It is believed that DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA. Using the techniques disclosed herein, greater than 40% of bombarded embryos may yield transformants.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty□ also is effective as a selection agent. Inhibition of GS in plants by PPT causes the rapid accumulation of ammonia and death of the plant cells.

The organism producing bialaphos and other species of the genus Streptomyces also Synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987) Brassica (De Block et al., 1989) and maize (U.S. Pat. No. 5,550,318). In previous reports, some transgenic plants which expressed the resistance gene were completely resistant to commercial formulations of PPT and bialaphos in greenhouses.

Another example of a herbicide which is useful for selection of transformed cell lines in the practice of the invention is the broad spectrum herbicide glyphosate. Glyphosate inhibits the action of the enzyme EPSPS, which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103. The best characterized mutant EPSPS gene conferring glyphosate resistance comprises amino acid changes at residues 102 and 106, although it is anticipated that other mutations will also be useful (PCT/WO97/4103).

To use the bar-bialaphos or the EPSPS-glyphosate selective system, bombarded tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/l bialaphos or 0.1-50 mM glyphosate will find utility in the practice of the invention. Tissue can be placed on any porous, inert, solid or semi-solid support for bombardment, including but not limited to filters and solid culture medium. Bialaphos and glyphosate are provided as examples of agents suitable for selection of transformants, but the technique of this invention is not limited to them.

It further is contemplated that the herbicide DALAPON, 2,2-dichloropropionic acid, may be useful for identification of transformed cells. The enzyme 2,2-dichloropropionic acid dehalogenase (deh) inactivates the herbicidal activity of 2,2-dichloropropionic acid and therefore confers herbicidal resistance on cells or plants expressing a gene encoding the dehalogenase enzyme (Buchanan-Wollaston et al., 1992; U.S. patent application Ser. No. 08/113,561, filed Aug. 25, 1993; U.S. Pat. No. 5,508,468; and U.S. Pat. No. 5,508,468; each of the disclosures of which is specifically incorporated herein by reference in its entirety).

Alternatively, a gene encoding anthranilate synthase, which confers resistance to certain amino acid analogs, e.g., 5-methyltryptophan or 6-methyl anthranilate, may be useful as a selectable marker gene. The use of an anthranilate synthase gene as a selectable marker was described in U.S. Pat. No. 5,508,468; and U.S. patent application Ser. No. 08/604,789.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants from bombarded immature embryos. In a similar fashion, the introduction of the C1 and B genes will result in pigmented cells and/or tissues.

The enzyme luciferase may be used as a screenable marker in the context of the present invention. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase, and manipulate those in real time. Another screenable marker which may be used in a similar fashion is the gene coding for green fluorescent protein.

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. A preferred growth regulator for such purposes is dicamba or 2,4-D. However, other growth regulators may be employed, including NAA, NAA+2,4-D or perhaps even picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber at about 85% relative humidity, 600 ppm CO2, and 25-250 microeinsteins m2 s-1 of light. Plants are preferably matured either in a growth chamber or greenhouse. Plants are regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants are preferably grown at about 19 to 28□C. After the regenerating plants have reached the stage of shoot and root development, they may be transferred to a greenhouse for further growth and testing.

Note, however, that seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10-5M abscisic acid and then transferred to growth regulator-free medium for germination.

Progeny may be recovered from transformed plants and tested for expression of the exogenous expressible gene by localized application of an appropriate substrate to plant parts such as leaves. In the case of bar transformed plants, it was found that transformed parental plants (RO) and their progeny of any generation tested exhibited no bialaphos-related necrosis after localized application of the herbicide Basta to leaves, if there was functional PAT activity in the plants as assessed by an in vitro enzymatic assay. All PAT positive progeny tested contained bar, confirming that the presence of the enzyme and the resistance to bialaphos were associated with the transmission through the germline of the marker gene.

C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

1. DNA Integration, RNA Expression and Inheritance

Genomic DNA may be isolated from callus cell lines or any plant parts to determine the presence of the exogenous gene through the use of techniques well known to those skilled in the art. Note, that intact sequences will not always be present, presumably due to rearrangement or deletion of sequences in the cell.

The presence of DNA elements introduced through the methods of this invention may be determined by polymerase chain reaction (PCR™). Using this technique discreet fragments of DNA are amplified and detected by gel electrophoresis. This type of analysis permits one to determine whether a gene is present in a stable transformant, but does not prove integration of the introduced gene into the host cell genome. It is the experience of the inventor, however, that DNA has been integrated into the genome of all transformants that demonstrate the presence of the gene through PCR™ analysis. In addition, it is not possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

2. Gene Expression

While Southern blotting and PCR™ may be used to detect the gene(s) in question, they do not provide information as to whether the gene is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and 14C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

### XIII. Plant Breeding

In addition to direct transformation of a particular plant genotype with a construct prepared according to the current invention, transgenic plants may be made by crossing a plant having a construct of the invention to a second plant lacking the construct. For example, a selected DNA comprising a suppressor of BYDV MP under the control of an inducible promoter or BYDV MP under the control of a suitable promoter can be introduced into a particular plant variety by crossing, without the need for ever directly transforming a plant of that given variety. Therefore, the current invention not only encompasses a plant directly regenerated from cells which have been transformed in accordance with the current invention, but also the progeny of such plants. As used herein the term “progeny” denotes the offspring of any generation of a parent plant prepared in accordance with the instant invention, wherein the progeny comprises a construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:

(a) plant seeds of the first (starting line) and second (donor plant line that comprises a transgene of the invention) parent plants;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

Backcrossing is herein defined as the process including the steps of:

(a) crossing a plant of a first genotype containing a desired gene, DNA sequence or element to a plant of a second genotype lacking said desired gene, DNA sequence or element;

(b) selecting one or more progeny plant containing the desired gene, DNA sequence or element;

(c) crossing the progeny plant to a plant of the second genotype; and

(d) repeating steps (b) and (c) for the purpose of transferring said desired gene, DNA sequence or element from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking said desired DNA sequence may be referred to as an unconverted genotype, line, inbred, or hybrid.

Introgression of Transgenes into Elite Varieties

Backcrossing can be used to improve a starting plant. Backcrossing transfers a specific desirable trait from a plant with one genetic background to another plant having a different genetic background which lacks that trait. This can be accomplished, for example, by first crossing a superior variety (for example, an inbred line) (recurrent parent) to a donor variety (non-recurrent parent), which carries the appropriate gene(s) for the trait in question, for example, a construct prepared in accordance with the current invention. The progeny of this cross first are selected in the resultant progeny for the desired trait to be transferred from the non-recurrent parent, then the selected progeny are mated back to the superior recurrent parent (A). After five or more backcross generations with selection for the desired trait, the progeny are hemizygous for loci controlling the characteristic being transferred, but are like the superior parent for most or almost all other genes. The last backcross generation would be selfed to five progeny which are pure breeding for the gene(s) being transferred, i.e. one or more transformation events.

Therefore, through a series a breeding manipulations, a selected transgene may be moved from one line into an entirely different line without the need for further recombinant manipulation. Transgenes are valuable in that they typically behave genetically as any other gene and can be manipulated by breeding techniques in a manner identical to any other corn gene. Therefore, one may produce inbred plants which are true breeding for one or more transgenes. By crossing different inbred plants, one may produce a large number of different hybrids with different combinations of transgenes. In this way, plants may be produced which have the desirable agronomic properties frequently associated with hybrids (“hybrid vigor”), as well as the desirable characteristics imparted by one or more transgene(s).

Marker Assisted Selection

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

In the process of marker assisted breeding, DNA sequences are used to follow desirable agronomic traits in the process of plant breeding (Tanksley et al., 1989). Marker assisted breeding may be undertaken as follows. Seed of plants with the desired trait are planted in soil in the greenhouse or in the field. Leaf tissue is harvested from the plant for preparation of DNA at any point in growth at which approximately one gram of leaf tissue can be removed from the plant without compromising the viability of the plant. Genomic DNA is isolated using a procedure modified from Shure et al. (1983), for example. In the technique of Shure et al (1983), approximately one gram of leaf tissue from a seedling is lyophilized overnight in 15 ml polypropylene tubes. Freeze-dried tissue is ground to a powder in the tube using a glass rod. Powdered tissue is mixed thoroughly with 3 ml extraction buffer (7.0 urea, 0.35 M NaCl, 0.05 M Tris-HCl pH 8.0, 0.01 M EDTA, 1% sarcosine). Tissue/buffer homogenate is extracted with 3 ml phenol/chloroform. The aqueous phase is separated by centrifugation, and precipitated twice using 1/10 volume of 4.4 M ammonium acetate pH 5.2, and an equal volume of isopropanol. The precipitate is washed with 75% ethanol and resuspended in 100-500 □1 TE (0.01 M Tris-HCl, 0.001 M EDTA, pH 8.0).

Genomic DNA is then digested with a 3-fold excess of restriction enzymes, electrophoresed through 0.8% agarose (FMC), and transferred (Southern, 1975) to Nytran (Schleicher and Schuell) using 10×SCP (20 SCP: 2M NaCl, 0.6 M disodium phosphate, 0.02 M disodium EDTA). The filters are prehybridized in 6×SCP, 10% dextran sulfate, 2% sarcosine, and 500 μg/ml denatured salmon sperm DNA and 32P-labeled probe generated by random priming (Feinberg & Vogelstein, 1983). Hybridized filters are washed in 2×SCP, 1% SDS at 65° C. for 30 minutes and visualized by autoradiography using Kodak XAR5 film. Genetic polymorphisms which are genetically linked to traits of interest are thereby used to predict the presence or absence of the traits of interest.

Those of skill in the art will recognize that there are many different ways to isolate DNA from plant tissues and that there are many different protocols for Southern hybridization that will produce identical results. Those of skill in the art will recognize that a Southern blot can be stripped of radioactive probe following autoradiography and re-probed with a different probe. In this manner one may identify each of the various transgenes that are present in the plant. Further, one of skill in the art will recognize that any type of genetic marker which is polymorphic at the region(s) of interest may be used for the purpose of identifying the relative presence or absence of a trait, and that such information may be used for marker assisted breeding.

Each lane of a Southern blot represents DNA isolated from one plant. Through the use of multiplicity of gene integration events as probes on the same genomic DNA blot, the integration event composition of each plant may be determined. Correlations may be established between the contributions of particular integration events to the phenotype of the plant. Only those plants that contain a desired combination of integration events may be advanced to maturity and used for pollination. DNA probes corresponding to particular transgene integration events are useful markers during the course of plant breeding to identify and combine particular integration events without having to grow the plants and assay the plants for agronomic performance.

It is expected that one or more restriction enzymes will be used to digest genomic DNA, either singly or in combinations. One of skill in the art will recognize that many different restriction enzymes will be useful and the choice of restriction enzyme will depend on the DNA sequence of the transgene integration event that is used as a probe and the DNA sequences in the genome surrounding the transgene. For a probe, one will want to use DNA or RNA sequences which will hybridize to the DNA used for transformation. One will select a restriction enzyme that produces a DNA fragment following hybridization that is identifiable as the transgene integration event. Thus, particularly useful restriction enzymes will be those which reveal polymorphisms that are genetically linked to specific transgenes or traits of interest.

### EXAMPLES

The following examples are offered by way of example, and are not intended to limit the scope of the invention in any manner.

### Example 1

### Exemplary Materials and Methods

The present example provides exemplary materials and methods to practice certain embodiments of the invention.

A. Yeast strains, plasmids and media Genotypes and sources of S. pombe strains and plasmids used in this study are summarized in Table 2. The MP gene was cloned into the leu1-selectable plasmid pYZ1N (Maundrell, 1993; Zhao et al., 1998a). Fission yeast cells carrying the leu1-selectable plasmid were maintained on agar plates of standard Edinburgh minimal medium (EMM: 3% KH-phtalate, 2.2% Na₂HPO₄, 5% NH₄Cl, 20% Glucose, pH 5.8, and Salt-, Mineral-, and Vitamin stock solution) supplemented with adenine and uracil at 75 μg/ml and thiamine added at 20 μM to repress MP expression from the nmt1 promoter as described previously (Maundrell, 1993; Zhao et al, 1996; Zhao et al., 1998a). Fission yeast cells were grown at 30° C. with constant shaking at 200 rpm except for the wee1-50 strain, which carries a temperature sensitive mutation and was grown at indicated temperatures. Agar plates were normally incubated at 30° C. for 3-5 days to obtain visible colonies.

TABLE 2
Arabidopsis thaliana, fission yeast strains and plasmids

| | plasmids | |
|---|---|---|
| Strains | Genotype and Characters | Source or Reference |

Arabidopsis thaliana

| | | |
|---|---|---|
| Wild type | wild type A. thaliana | Hartung et al., 2003 |
| TS-4-12 | MP::GFP transgenic A. thaliana | This study |

Schizosaccharomyces prombe:
Wild type:

| | | |
|---|---|---|
| SP223 | wild-type, h⁻, ade6, leu1, ura4 | David Beach |

Checkpoints mutants:

| | | |
|---|---|---|
| rad3 | h⁻, leu1, ura4, rad3-136 | Howard Lieberman |
| chk1 | h⁻, leu1, ura4, chk1::ura4+ | David Beach |
| cds1 | h⁻, leu1, ura4, cds1::ura4+ | Paul Russell |
| chk1/cds1 | h⁻, leu1, ura4, chk1::ura4+, | Paul Russell |
| | cds1::ura4+ | |

Mutants in mitotic regulators:

| | | |
|---|---|---|
| cdc2-1w | h⁻, leu1, ura4, cdc2-12 | Paul Nurse |
| cdc2-3w | h⁻, leu1, ura4, cdc2-3w | Paul Russell |
| wee1-50 | h⁻, leu1, ura4, cdc25::ura4+, | Paul Russell |
| | wee1-50 | |

Protein phosphatase 2A and PP1 mutants:

| | | |
|---|---|---|
| ppa2 | h⁻, leu1, ura4, ppa2::ura4+ | Mitsushiro Yanagida |
| pab1 | h⁻, leu1, ura4, pab1::ura4+ | Mitsushiro Yanagida |
| ppe1 | h⁻, leu1, ura4, ppe1::ura4+ | Mitsushiro Yanagida |

Plasmids:

| | | |
|---|---|---|
| pYZ1N | derivative of pREP1N | Zhao et al., 1998b |
| pYZ1N-MP | BYDV MP cloned in pREP1N | This study |
| pYZ4N-MP | BYDV GFP-MP fusion | This study |

### B. Molecular Cloning and Gene Induction of MP in S. Pombe

The MP (P4) gene of the BYDV viral isolate GAV was cloned into fission yeast expression vector pYZ1N or pYZ4N for GFP fusion as previously described (Zhao et al., 1998a). This vector contains an inducible nmt1 (no message in thiamine) promoter, which can be repressed or induced in the presence or absence of thiamine (Maundrell, 1993). Insertion of the MP gene into pYZ vectors was confirmed by extracting DNA from individual E. coli colonies containing the plasmids and subsequently analyzing by restriction mapping and PCR. The complete wild-type nucleotide sequence of the MP gene was confirmed by DNA sequencing using an ABI 377 automated sequencer. For MP induction in liquid medium, cells containing the MP plasmid were first grown to stationary phase in the presence of 20 μM thiamine. Cells were then washed three times with distilled water, diluted to a final concentration of approximately 2×105 cells/ml in 10 ml of the appropriately supplemented EMM medium with or without thiamine.

C. Cell growth and colony formation on agar plate S. pombe cells with or without MP gene expression were prepared as above for MP gene induction and grown at 30° C. with shaking (200 rpm). An aliquot of each culture was collected at sequential time intervals as indicated; the number of cells per milliliter was counted using a hemacytometer. For determining ability of cells to form colonies on agar plates, a loopful S. pombe cells were streaked onto EMM-selective agar plates with (MP-off) or without (MP-on). Agar plates were then incubated for 3-5 days at indicated temperatures prior to documentation.

D. Determination of cell cycle G2/M arrest The strongly regulated nmt1 promoter (Maundrell, 1993; Zhao et al., 1996) allows expression of the MP gene to be turned OFF or ON simply by adding or removing thiamine from the growth media. Using this inducible MP gene expression system, the effect of MP on cell cycle G2/M regulation was measured in fission yeast by using several different procedures including measurement of DNA content by flow cytometry, determination of Cdc2 phosphorylation status by immunoblots analysis, cell elongation by forward scatter analysis, or direct visualization by microscopy, for example. A single nucleated S. pombe cell that becomes longer than normal, i.e., >8-12R, upon DNA damage, is normally an indication of cell cycle G2/M arrest, which is commonly known as the “cdc phenotype” (Lee and Nurse, 1988; Nurse et al., 1976). This method has also been successfully used to monitor HIV-1 Vpr-induced G2 arrest in S. pombe (Masuda et al., 2000; Zhao et al., 1996). A statistical two-sided t-test was used to determine the significance of cell length measured in MIP-expressing vs MP-repressing cells. Another way to examine the effect of MP on cell length is to use forward scatter analysis from flow cytometry in which cell elongation of MP-on and MP-off cells are measured in a population of 10,000 cells (Zhao et al., 1996; Zhao et al., 1998b). Increased phosphorylation of Cdc2 is another indication of cell cycle G2/M arrest. To measure Cdc2 phosphorylation, cell extracts were prepared as described by Kovelman and Russell (Kovelman and Russell, 1996). Briefly, cell extracts were prepared with modified RIPA buffer with complete Mini protease inhibitors; Na₃VO₄ was added using a minibeadbeater.

The protein concentrations were determined by using the BCA protein colorimetric assay (Pierce). Equal amounts of cell extracts from fission yeast cells with or without MP were separated by size using 4-15% tris-HCl gradient gel with tris-glycine running buffer. After transferring proteins to nitrocellulose membrane, anti-Cdc2 (Upstate) or anti-phospho Cdc2 (Cell Signaling) antibody was used following manufacturer's instructions. Proteins reacted to the antibodies were revealed by using the SuperSignal West Pico Chemiluminescent Substrate Kit (Pierce) and detected on Xray films. To quantify the degree of MP-induced G2, flow cytometric analyses were used to measure cell cycle profile as indicated by DNA content in cell cultures grown in low nitrogen media (Zhao et al., 1996). Cells are grown in low nitrogen to synchronize S. pombe cells in G1 phase of the cell cycle (Alfa et al., 1993). Potential G2 induction by MP is then determined by comparing the percentage of the synchronized G1 cell population in MP-repressed cells with that in the MP-expressing cells (Elder et al., 2000). Thus, the extent of MP-induced G2/M arrest as measured by flow cytometry is expressed as the percentage of G1 cells in the MP-off cultures that shift to G2 when MP is expressed. To compare the extent of MP-induced G2 arrest in different genetic backgrounds, MP-induced G2 arrest in the mutant cells was normalized to the value for the wild-type strain done in parallel in each experiment so that wild type and a mutation not affecting MP-induced G2 arrest have a value of 100%.

E. Fluorescence and Confocal Microscopy A Leica fluorescence microscope DMR equipped with a high performance CCD camera (Hamamatsu) and OpenLab software (Improvision, Inc., Lesington, Mass.) was used for all imaging analyses. For the observation of green fluorescent protein, a Leica L5 filter was used, which has an excitation of 480/40 (460-500 nm) and emission of 527/30 (512-542 nm). For DNA straining, cells were counterstained in fission yeast with 1 μg/ml DNAbinding fluorescent probe 4′,6-diamidino-2-phenylindole (DAPI), which was observed with a Leica A8 filter with an excitation of 360/40 (340-380 nm) and emission of 470/40 (450-490 nm). Calcofluor (Sigma F6259), a chitin-specific dye, was used as a fluorescent cell wall marker to identify septum in dividing S. pombe cells. The procedures for cell preparation and staining using Calcofluor and DAPI have been described previously (Alfa et al., 1993). For DNA staining in A. thaliana, propidum iodine was used and observed with a Leica LP590 filter with an excitation of 515-5.60 nm and emission of 620 nm).

To examine cell morphology in the root meristemic regions, the seeds of the wild type and transgenic strains of Arabidopsis thaliana were germinated on Murashige and Skoug basal medium (MS media) for three days at 4° C. The germinated seedlings were then grown vertically on fresh MS media containing 0.5 μM estradiol at 23° C. in a lighted growth chamber. After three days, the root tips (around 5 mm in length) were collected on ice for confocal microscopy. Each root tip was placed on glass slide with 15 μl of sterile water and was examined using a confocal microscope (Olympus FV500) without further treatment.

F. Generation of MP transgenic A. thalina. Determination of ploidy of A. thaliana cells To prepare samples for determination of genomic constitution in root tips, the seeds of the wild type and transgenic strains of Arabidopsis thaliana were germinated on MS media for three days at 4° C. The germinated seedlings were then grown vertically on fresh MS media containing 0.5 μM estradiol at 23° C. in a lighted growth chamber. After confirming the expression of the MP-GFP fusion protein in the transgenic strain using confocal microscopy, the root tips (around 5 mm in length) were collected from both the transgenic and wild type strains on ice. They were then fixed in an ethanol/glacial acetic acid mixture (3:1) for two days at 25° C. The fixed root tips were squashed onto clean glass slide, which was immediately placed in a −70° C. freezer to promote the adherence of squashed cell materials to the glass surface.

Chromosome numbers of A. thaliana in MP-producing transgenic plant were determined by fluorescence in situ hybridization (FISH) detecting the 5S rDNA loci as previously described (Fransz et al., 1998; Murata et al., 1997). Briefly, the hybridization probe was prepared by nick translation using biotin-16-dUTP (Roche). The hybridization signal was detected using avidin conjugated fluorescein (Roche). The slides were examined under an epifluorescent microscope (Leica). The images were captured using a Spot CCD digital camera (Diagnostic Instrument Inc., USA).

### Example 2

### Expression of BYDV MP Inhibits Cell Proliferation and Causes Growth Retardation in S. Pombe and A. Thaliana

An exemplary fission yeast model system was developed to study the effect of BYDV viral proteins on basic cellular functions. Each one of the BYDV gene was cloned and expressed in an inducible fission yeast expression vector pYZ1N, which is under the control of an inducible nrmt1 (no message in thiamine) promoter (Maundrell, 1993; Zhao et al., 1998a). Expression of the MP (P4) gene was found to inhibit cell proliferation of S. pombe cells (FIG. 1A). For example, time course experiments were conducted to compare growth rate of S. pombe cells expressing MP (MP-on) with those cells in which MP gene expression was suppressed (MP-off). Both types of cells were grown in plasmid-selective EMM medium with or without thiamine. Both cultures were initiated at early log phase (actively growing stage) with an initial concentration of approximately 2×10⁵ cells per ml. An aliquot of each culture was collected at different time points and counted for cell growth. The MP-off culture reached stationary phase after about 28 h, indicating normal cellular growth of S. pombe, with a doubling time of about 4 h. In contrast, the MP-induced cells displayed dramatic growth delay. More than one log difference in cell growth was observed between the MP-induced and MP-repressed fission yeast cells (FIG. 1A-a). Similar growth dynamics between the MP-repressed and MP-inducing S. pombe cells were also observed when the MP gene was fused with GFP at its N-terminal end (data not shown). Consistently, little or no colony formation was observed on agar plates when the MP gene was expressed (FIG. 1A-b, right. By contrast, normal colony formation was seen when the MP gene was suppressed by plating cells on thiamine-containing (MP-off) agar plate (FIG. 1A-b, left). Together, these data show that BYDV MP inhibits cell proliferation of S. pombe cells.). This discovery forms the basis of an embodiment of the invention directed to a screening assay using cells transformed with the MP gene to identify a compound that affects MP gene expression. In a preferred embodiment the compound identified in the live cell screening assay comprise a molecule that inhibits MP expression or activity (e.g., MP suppressor) Such MP suppressors can thereby control BYDV infection in plants. In such an assay, MP-transformed yeast cells have improved viability, normal cell size, and the ability to form colonies if the test agent suppresses MP expression or interferes with MP function.

### Example 3

### BYDV MP Reduces Plant and Root Growth of A. Thaliana Cells

To determine whether MP has similar effect on plant growth, a series of transgenic A. thaliana cells that specifically produce MP protein upon gene induction was created using a XVE inducible system (Zuo et al., 2000). To facilitate monitoring of MP production in plants, the MP gene was fused at its C-terminal end with green fluorescent protein (GFP). To ascertain whether the MP::GFP effect on plant growth is due to MP and not GFP or protein fission, two additional constructs were also established as controls. A GFP only control was created to test the potential effect of GFP on plant growth. A MP::His fission, in which the His tag (9 aa) is much smaller than GFP (238 aa), was generated as a control for fusion protein. Seeds carrying different transgenic constructs were germinated under MP-inducing conditions as previously described (Parker et al., 1993). Consistent with the observations in S. pombe, significant reduction of plant growth was observed in MP::GFP-expressing A. thaliana plants in comparison with the wild type plant (FIG. 1B). In the control plants, transgenic A. thaliana with the GFP gene alone did not affect plant growth. MP fused with a His-tag had a similar growth retardation effect on plant growth to the MP-GFP fusion product. This shows that the retardation effect on plant growth is due to MP instead of the protein fusion. To test whether MP expression also affects root growth, the expression of MP in roots was measured. As shown by cross-section of a root tip in FIG. 1C-a, MP was produced predominantly in the root stem with little expression in the root hairs. Similar to what was observed in the up-growing plants, expression of MP::GFP but not GFP alone significantly reduced length of the roots (FIG. 1C-b). For example, roots in the wild type plants grow from about average of 3.2 to about 5.2 cm in length 5 and 9 days after seed germination. By contrast, only an average of 1.1 to 1.5 cm long roots were seen in MPexpressing plants (FIG. 1C-c).

Together these data show that expression of the BYDV MP gene similarly inhibits cell proliferation in both S. pombe and A. thaliana, indicating that this is a highly conserved effect of viral MP. The ability of the MP gene to inhibit cell growth in A. thaliana forms the basis of an embodiment of the invention directed to a screening assay using a plant cell transformed with the MP gene to identify a compound that affects MP gene expression or function. In a preferred embodiment the compounds identified in the live cell screening assay are molecules that suppress MP expression or activity. Such suppressors can thereby reduce or control the adverse side effects of BYDV infection in plants. In such an assay, MP-transformed plant cells live, divide and form colonies if the test agent suppresses MP expression or interferes, blocks, or otherwise interferes with MP function.

### Example 4

### Cell Elongation with Enhanced 2N (G2) DNA Content Induced by BYDV MP in S. Pombe or A. Thaliana

The effect of MP on cell morphology of fission yeast cells was evaluated. In the thiamine-containing growth medium (MP gene expression is OFF; FIG. 2A, left), fission yeast cells with MP plasmid are of normal length [10.4±0.2μ; (Zhao and Lieberman, 1995)]. In contrast, the mean cell length of the MP-expressing strain is 12.6±0.4μ (standard error of the mean) and is statistically significant at the p<0.0001 level when compared to cell length of the wild-type (FIG. 2A-a). In addition to an increased mean cell length in the MP-expressing cell population, some of the cells are longer than 18μ while no such cells are seen in the MP-repressing cells (FIG. 2A-b). An alternative method to determine cell length in a large cell population is to use forward scatter analysis by which cell elongation and gross enlargement of the MP-expressing cells were both detected from a population of 10,000 cells (FIG. 2A-c, top). Increased cell length in fission yeast often indicates a cell cycle G2/M arrest and is commonly known as the “cdc phenotype” (Lee and Nurse, 1988; Nurse et al., 1976). Thus, the ability of MP to induce cell cycle arrest in S. pombe cells was assessed by flow cytometry analysis. In standard EMM, about 70% of S. pombe cells normally reside in the G2 phase of the cell cycle (Alfa et al., 1993). In order to test potential cell cycle G2/M arrest, S. pombe cells were synchronized by growth in low nitrogen medium for accumulation of G1 cells (Alfa et al., 1993). If MP induced G2 arrest, one would expect a shift of the predominantly G1 cell population to G2. As results, expression of BYDV MP arrested S. pombe cells in the G2 (FIG. 2A-c, bottom). In the G1-enriched cell population, the cells started to switch from G1 to G2 phase as early as 24 h post-induction. By 40 hr, a significant portion of cells rested in the G2 phase (FIG. 2A-c, bottom right). As an additional control, the pYZ1N vector was also used in this test, and no G2 arrest was observed under either gene-inducing or gene-repressing conditions (data not shown). DNA larger than diploid (2N) was also noted in the MP-expressing cells, which suggests potential aneuploidy of the cells (This issue was further examined in later section).

Together, results of these observations showed that BYDV MP induces cell cycle G2/M arrest in S. pombe. It is technically difficult to determine whether MP has similar cell cycle effect on plant cells as shown in S. pombe simply because suspension plant cells are difficult to obtain. However, if a similar effect were observed in plants as in S. pombe, one would expect comparable changes in cell morphology including cell elongation and gross enlargement. Based on this rationale, cell morphology in the root meristematic regions in the wild type and the MP-transgenic strains was compared (FIG. 2B). Major differences were found in the cells in the meristematic zone (left panel) between the wild type and transgenic strains (right panel). In the wild type strain, the cells in the meristematic zone (indicated by arrows) were compact and their size was regular, whereas in the transgenic strain the cells from the same zone (indicated by open arrow heads) were longer and their size was much larger (FIG. 2B, right).

### Example 5

### Hyper-Phosphorylation Of Cyclin-Dependent Kinase CDC2 by MP and Suppression of MO-Induced Cell Cycle G2/M Arrest by CDC2-1W and Wee1-50

Cell cycle G2/M transition is a highly regulated cellular process, in which the cyclindependent kinase Cdc2 plays a pivotal role. In all eukaryotes, progression of cells from G2 phase of the cell cycle to mitosis requires activation of Cdc2 (Morgan, 1995). Typically, entry to mitosis is regulated by phosphorylation status of Cdc2, which is phosphorylated by Wee1 kinase during G2 and rapidly dephosphorylated by the Cdc25 phosphatase to trigger entry to mitosis (Gould and Nurse, 1989; Krek and Nigg, 1991; Morgan, 1995; Norbury et al., 1991). To determine whether MP exerts its cell cycle effect directly on Cdc2, phosphorylation status of Cdc2 kinase was measured under the MP-on and MP-off conditions using immunoblot analyses (FIG. 3A). Two closely spaced bands of approximately 34 kD reacted to the anti-Cdc2 antibody under MP-repressing condition (FIG. 3A, top left). The upper band is known to correspond to the phosphorylated inactive form of Cdc2 (Hayles and Nurse, 1995), which was further shown by an antibody specifically against the phosphorylated form of Cdc2 [(FIG. 3A, middle panel; (Hayles and Nurse, 1995)]. The lower band has been shown to represent the dephosphorylated active form of Cdc2 (Hayles and Nurse, 1995). Both bands were clearly visible in the MP-off cells. In contrast, Cdc2 became predominantly phosphorylated in MP-on cells after 24 hr gene induction (FIG. 3A, top right), suggesting MP promotes hyper-phosphorylation of Cdc2.

Earlier studies have shown that tyrosine phosphorylation of Cdc2 on residue 15 is specifically responsible for inactivation of Cdc2 during G2/M transition (Gould and Nurse, 1989; Krek and Nigg, 1991). To test whether MP induces G2/M arrest by interfering with the phosphorylation of Tyr15 on Cdc2, the MP gene was expressed in two non-conditional cdc2 mutants (cdc2-1w and cdc2-3w). The cdc2-1w and cdc2-3w alleles are resistant to the effects of Tyr15 phosphorylation. The cdc2-1w mutation results in partial resistance of Cdc2 to phosphorylation by the Wee1 kinase (Enoch and Nurse, 1990); the cdc2-3w allele has the same phosphorylation levels as wild-type but retains partial Cdc2 kinase activity presumably due to in part its inability to be removed by Cdc25 (Enoch and Nurse, 1990; Gould et al., 1990; MacNeill et al., 1991). As consequences, the Cdc2 kinase in both mutants is overactive leading to premature mitosis and smaller cells compared to wild-type Cdc2 (Enoch and Nurse, 1990).

Expression of MP in these two cdc mutant strains indicated that the cdc2-1w but not cdc2-3w suppressed MP-induced cell elongation (FIG. 3B, middle panel). For example, MP expression in wild-type cells resulted in cell length of 12.31+0.41μ, which is significantly (p<0.0001) longer than the MP-repressing cells (7.9+0.0111). In contrast, MP expression in cdc2-1w cells completely blocked MP-induced cell elongation as cell length in MP-expressing cells (5.80+0.12μ) was not significant difference (p=0.16) to the MP-repressing cells (5.92+0.13μ). A small difference was observed in cdc2-3w strain between the MP-expressing (9.13+0.38μ) and MP-repressing (6.35+0.14μ) cells. Statistical analysis showed, however, this small difference between those two cell populations was significant (p<0.0001).

The suppressive effect of cdc2-1w on MP implicates potential involvement of Wee1 kinase in MP-induced G2/M arrest. To examine this possibility, MP gene was expressed in a temperature sensitive (ts) wee1-50 mutant strain. This strain grows normally under low permissive temperature at 25.5° C. However, cells show “wee” phenotype due to inhibition of the Wee1 kinase at high non-permissive temperature (Lundgren et al., 1991). As shown in FIG. 3C-ab, expression of MP in wee1-50 significantly reduced MPinduced cell elongation and restored their abilities to form colonies on agar plates. For example, differences of the cell length between the MP-expressing and MP-repressing wee1-50 cells became increasingly smaller as temperature increased from permissive (25° C.), semi-permissive (30° C.) and to non-permissive (35.5° C.) (FIG. 3C-a).

Consistently, cells also restored their abilities to form colonies on agar plates when MP was expressed in wee1-50 instead of the wild type cells (FIG. 3C-b). These data augmented the observation in the cdc2-1w mutant strain and confirmed suppression of MP-induced G2/M arrest in these genetic backgrounds (FIG. 3B-C). Therefore, MP induces G2/M arrest at least in part through the Wee1 kinase in fission yeast. The residual difference of cell length in the cdc2-3w mutant strain indicates that Cdc25 might also play a role in MP-induced cell cycle arrest. However, the deletion effect of cdc25 on MP was not amenable to testing, as cdc25 deletion is lethal due to terminal G2 arrest. The role for Cdc25 in MP-induced G2/M arrest was characterized by using a direct in vivo assay for Cdc25 activity (Furnari et al., 1999; Furnari et al., 1997). This assay uses a strain with a mik1 deletion and the ts wee1-50 mutation so that both kinases are inactive at the restrictive temperature. Under these conditions, phosphate is no longer added to Tyr15 of Cdc2 so that the activity of the Cdc25 phosphatase alone controls the level of Tyr15 phosphorylation. Since removal of the inhibitory phosphate from Tyr15 leads to mitosis and cell division, the removal of phosphate from Tyr15 can be followed by increased septation of S. pombe cells as the cells divide. This assay has been used to show that both the DNA damage and DNA replication checkpoints inhibit Cdc25 (Furnari et al., 1999; Furnari et al., 1997). When this in vivo Cdc25 assay was applied to MP, it showed that MP delays septation in a similar kinetics as cells without MP (FIG. 3D). Thus, it is less likely that Cdc25 plays an important role in MP-induced cell cycle arrest.

### Example 6

### MP Does Not Use the DNA Damage and DNA Replication Checkpoints During Induction of G2/M Arrest

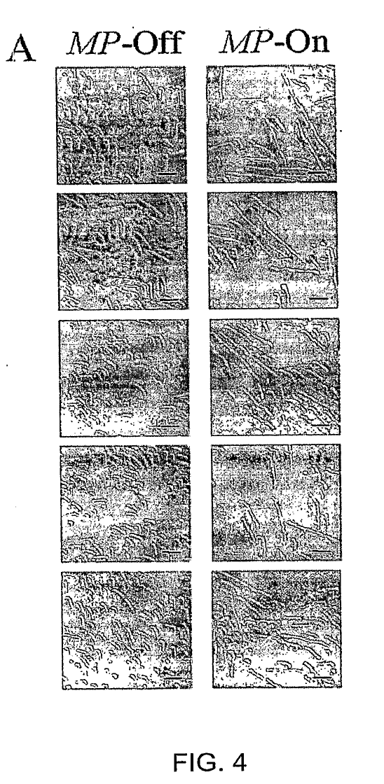

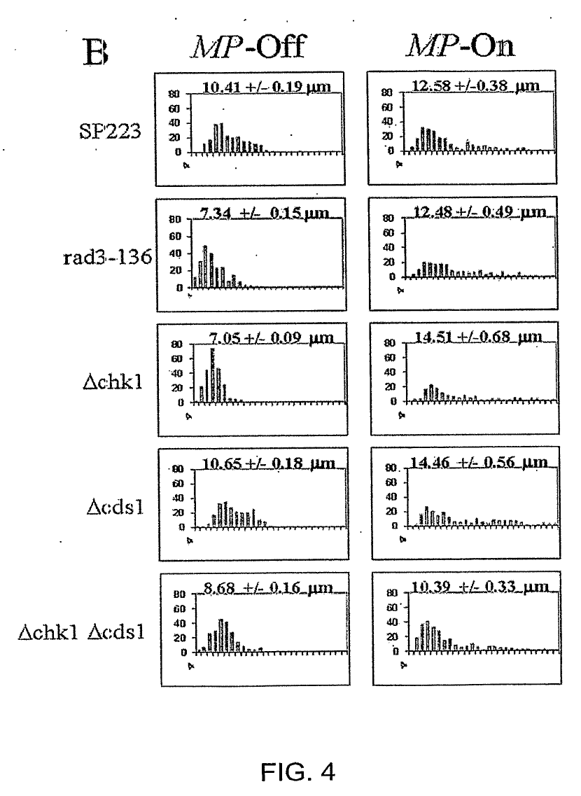

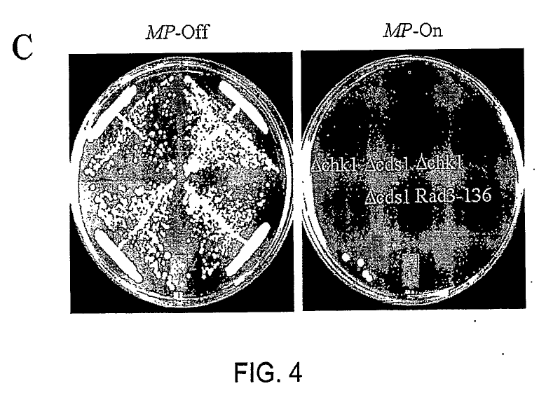

Since MP and the checkpoints for DNA damage and DNA replication all induce cell cycle arrest through phosphorylation of Tyr15 on Cdc2 (Chen et al., 2000; Nurse, 1997; Rhind and Russell, 1998), MP might induce G2 arrest through one of the checkpoint pathways. To examine these possibilities, MP was expressed in a rad3-136 mutant strain. Rad3 acts as a sensor protein early in both checkpoint pathways, and a rad3 mutation blocks the induction of G2 arrest by DNA damage or inhibition of DNA synthesis (al-Khodairy and Carr, 1992). However, rad3 mutation was not able to block MP-induced G2/M arrest as MP induced the same level of cell elongation as in the wild type cells (FIG. 4A-B). Similarly, MP also blocked colony formation on agar plate in rad3 mutant strain (FIG. 4C). To explore the possibility that MP acts downstream of these early checkpoint genes, MP was expressed in strains mutant for the Chk1 and Cds1 kinases.

These two kinases were thought to be the last regulatory genes specific for the DNA damage or DNA replication checkpoint, respectively (Boddy et al., 1998; Furnari et al., 1997; Zeng et al., 1998). Similar to rad3, expression of MP in a chk1 or cds1 deletion strain induced levels of cell elongation and inhibition of colony formation similar to that in wild-type (FIG. 4A-C). However, since chk1 is not only involved in the DNA damage checkpoint but also plays a role in the DNA replication checkpoint, only a chk1/cds1 double-mutant strain completely prevents G2 arrest in response to inhibition of DNA replication (Boddy et al., 1998). Invariably, expression of MP showed the inhibitory phenotypes similar to that in wild-type in the chk1/cds1 strain. Thus, none of these mutations defective in the early or late steps of the checkpoint pathways significantly reduced MP-induced G2 arrest, indicating that MP must use an alternative pathway to induce G2 arrest.

### Example 7

### Suppression of MP by PAB1 or PPE1 Gene Deletion

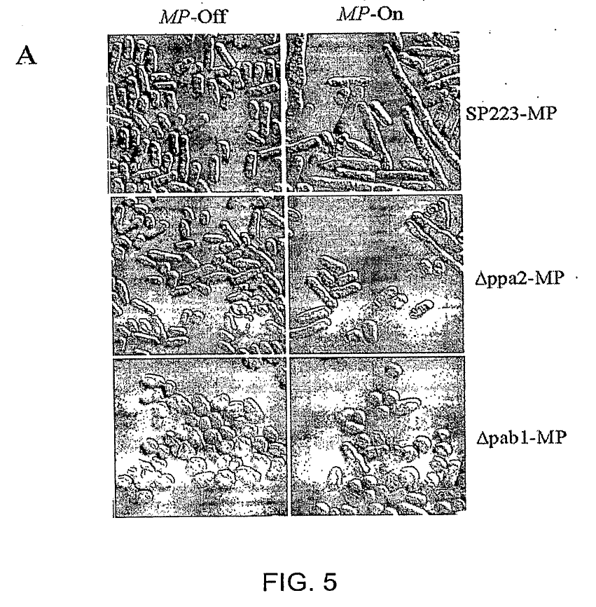

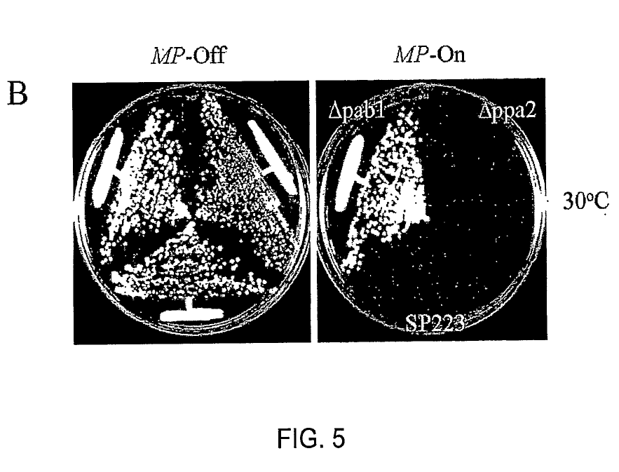

Previously, studies demonstrated that other viral proteins such as HIV-1 Vpr or adenovirus E4Orf4 induces cell cycle G2 arrest by modulating through PP2A (Elder et al., 2000; Kornitzer et al., 2001). The inventors were interested in whether MP also modulates PP2A or PP2A-like enzymes during induction of cell cycle G2/M arrest. To examine these possibilities, a S. pombe strain mutant for the catalytic subunit of PP2A (ppa2) was transformed with the MP expression plasmid, and the effect of MP expression on cell elongation and colony forming ability was determined. In fission yeast, there are two genes (ppa1 and ppa2) that encode the catalytic subunit of PP2A. However, only the mutant effect of ppa2 was tested, as it represents approximately 90% of the catalytic subunit made in a normal fission yeast cell (Kinoshita et al., 1990). However, ppa2 deletion was not able to block MP-induced G2/M arrest as MP induced similar level of cell elongation as in the wild type cells (FIG. 5A). Similarly, MP also blocked colony formation on agar plate in ppa2 deletion strain (FIG. 5B). Because the regulatory subunit of PP2A (pab1) normally determines the substrate specificity, it was then examined whether a mutation in a regulatory subunit of PP2A could affect MP-induced G2 arrest. Interestingly, expression of MP in pab1 deletion strain showed different results in the MP effects on cell length and colony formation. There was no significant difference in cell length between MP-expressing and MP-repressing cells (p=0.19), indicating pab1 deletion may have blocked MP-induced cell elongation (FIG. 5A). However, Δpab1 cells expressing MP did not form visible colonies; on agar plates, indicating inability of this mutant in suppressing the MP effect on colony formation (FIG. 5B). The role of PP2A-like enzyme in MP-induced G2 arrest was also examined. S. pombe strain with a gene deletion mutation for this enzyme (ppe1) was examined (Kinoshita et al., 1990).

Deletion of ppe1 suppressed the effects of MP on both cell elongation and colony forming ability (FIG. 5A-B). For instance, no significant difference in cell length was observed between the MP-expressing and MP-repressing Δppe1 cells (p=0.86; FIG. 5A, bottom row). In contrast to the wild type control cells, Δppe1 cells expressing MP formed colonies to the similar extent as those cells without MP (FIG. 5B). Therefore, MP functionally interacts with PP2A-like enzyme during induction of cell cycle arrest, in certain embodiments of the invention.

### Example 8

### Mitotic Abnormality Caused by MP in S. Pombe and A. Thaliana

Protein phosphatase 2A or PP2A-like enzymes are highly conserved among all eukaryotes (Goshima et al., 2003; MacKintosh et al., 1990). Earlier studies in budding yeast showed that CDC55, fission yeast homologue of Pab1, plays a dual role in inhibitory phosphorylation of CDC28 (fission yeast homologue of Cdc2) during G2/M transition and the mitotic kinetochore/spindle checkpoint (Minshull et al., 1996; Wang and Burke, 1997). Role of CDC55 in mitosis is independent of DNA damage and replication checkpoints because cdc55 mutants showed normal sensitivity to gamma radiation and hydroxyurea (Wang and Burke, 1997). Moreover, a cdc28 mutant that lacks inhibitory phosphorylation sites on Cdc28 allows spindle defects to arrest cdc55 mutants in mitosis with active MPF and unseparated sister chromatids (Minshull et al., 1996). Since deletion in S. pombe pab1 suppressed MP-induced cell elongation (FIG. 5A), this finding combined with those earlier reports led the inventors to surmise that MP has added impact on mitosis, in certain embodiments. Three evidences supported the embodiment. First, results of the flow cytometric analyses in MP-expressing cells showed significant portion of DNA detected were larger than 2N, indicating possible aneuploidy of the cells (FIG. 2A-c. bottom), which is often generated as a result of mitotic abnormality due to unequal segregation of chromatids. Second, S. pombe Ppe1 has recently been shown to play a specific role in equal chromosome segregation in fission yeast (Goshima et al., 2003), and thirdly, ppe1 deletion completely suppressed the MP effects (FIG. 6A-b, B-b).

To test whether MP causes any defect during mitosis, MP-expressing cells were co-stained with DAPI to see the nuclei and Calcofluor to visualize septum formed during cytokinesis (FIG. 6A). In MP-repressed cells, normal nuclear morphology with equal segregation was observed after formation of septum (FIG. 6A-a-i). In contrast to these normal patterns of nuclear morphology and chromosome segregation, MP-expressing cells showed significant mitotic abnormality including unequal chromosome segregation (FIG. 6A-a-ii-iv) and “cut” phenotype [FIG. 6A-a-v-vi; (Funabiki et al., 1996)]. Twenty hours after MP gene induction, approximately 25% (n=250) of the total cell population displayed septa, in which 23.85% showed abnormal nuclear separation in septated cells (FIG. 6A-a). A small increase (27.92%) was observed at 24 hr and maintained at a similar level thereafter presumably due to the cell cycle arresting effect. In addition, 1.8% to 4.16% (n=250) of the total cell population also displayed the “cut” phenotype when these phenotype were screened for from 20 to 32 hr after gene induction (FIG. 6Aa-v-vi). It was previously shown that Ppe1 involves in equal chromosome segregation in fission yeast (Goshima et al., 2003), However, deletion in ppe1 suppressed MP (FIG. 5A-b). It seemed that the data was in conflict with that of the earlier report (Goshima et al., 2003). In attempting to resolve this discrepancy, percentage of unequal chromosome segregation in the MP-expressing Δppe1 cells was also measured. Surprisingly, the nuclear pattern appeared to be normal, i.e., similar to the MPrepressed wild type cells (FIG. 6A-a-i). This observation was consistent with the suppressive effect of Δppe1 observed on MP (data not shown).

One of the possible explanations for MP suppression by Δppe1 is the lack of Ppe1 may have potential negative effect on MP. Since Ppe1 binds to chromatin in the nucleus (Goshima et al., 2003), this negative effect could include its effect on cellular localization of MP. The subcellular localization of MP was examined and tested whether MP causes unequal nuclear segregation by direct association with the nuclei. A GFP-MP fusion plasmid was constructed and expressed in the wild type S. pombe cells. GFP-MP protein appeared to be in the nucleus as it co-stained with DAPI (FIG. 6B-a). Similar to MP without GFP fusion, it also caused unequal nuclear segregation and associates mostly with the nuclei regardless of their localizations (FIG. 6B-a). Interestingly, in contrast to nuclear localization of GFP-MP in wild type cells, GFP-MP proteins were dispersed evenly throughout the Δppe1 cells, and nuclear distribution returned back to normal like wild type (FIG. 6B-b).

To determine whether MP has a similar effect on chromosome segregation in plant cells, chromosomal ploidy of root hair cells was determined by using fluorescence in situ hybridization (FISH). There are six 5S rDNA loci in the diploid genome of a normal wild type Arabidopsis plant (Murata et al., 1997). The six loci can be visualized by FISH using 5S rDNA specific probe. In a FISH experiment, any Arabidopsis root tip cells showing more or less than six rDNA loci may have abnormal genome constitution. As shown in FIG. 6C and FIG. 7A, in the wild type strain the great majority of root tip cells examined were at interphase with six rDNA loci (indicated by arrowheads). About 3% of cells were at mitosis (FIG. 7B), some of which were at metaphase with closely spaced 5S rDNA loci on sister chromatids (FIG. 6C-c, 7A, bottom). Note that the replicated chromosomes aligned regularly along the equatorial plate. In the transgenic strain, about 1% of cells were also found at metaphase (FIG. 6C-c. 7A, bottom). However, the replicated chromosomes distributed irregularly. In about 3 to 4% of cells, 12 rDNA loci were detected (FIG. 6C-c, right). Because these cells were at interphase, they were therefore tetraploid rather than diploid. To quantify potential differences of abnormal mitotic cells between the wild type and MP-transgenic plants, the proportion of root tip cells showing abnormal 5S rDNA loci was measured. As shown in FIG. 7B, percentage of abnormal 5S rDNA loci was significantly higher in the transgenic strain than that in the wild type strain. Note that the presence of some cells showing abnormal 5S rDNA loci in the wild type strain is most likely caused by incomplete adherence of cell materials to the slide during the FISH experiment.

To further examine potential association of MP with the nucleus in A. thaliana cells, localization of MP-GFP was compared with nuclear DNA stained with propidium iodine. As shown in FIG. 6D, similar to what was found in fission yeast cells, localization of MP co-localizes in many cells with the nuclei. Even though MP and PI staining were seen both in the root stem and root hair, notably, however, not all of the MP proteins associated with the nuclei, or vice versa (FIG. 6D).

### Example 9

### Searches for BYDV MP Suppressors in Yeast

To search for BYDV MP suppressors for BYDV MP-induced growth inhibition and cell killing, a fission yeast, such as S. pombe or S. cerevisiae, expression cDNA library is transformed into a genetically engineered yeast strain that carries a single integrated copy of BYDV MP. Using a genetically engineered yeast to measure BYDV MPinduced cell killing is based on the fact that expression of BYDV MP in fission yeast prevents formation of colonies on agar plate due to rapid cell death induced by the BYDV MP expressed protein. Thus, the criterion used to identify a MP suppressor (i.e., phenotypically can be BYDV MP-induced cell death), for example, is the ability of a fission yeast transformant to form normal size colonies on the BYDV MP-expressing agar plate.

Suppression effects of the identified cDNA clone is confirmed by re-introducing the corresponding cDNA-carrying plasmid back into the parental strain, and the putative gene function is identified by a homology search (such as BLAST) of S. pombe databases, for example, such as may be found on National Center for Biotechnology Information's GenBank® database, for example.

The principle of this invention allows searching for BYDV MP suppressors in different organisms such as bacteria or other yeast. The BYDV MP suppressors could not only include proteins as described above but also any other inhibitory compounds such as small molecules or natural compounds.

Other and further aspects, features, and advantages of the present invention will be apparent from the following description of the presently preferred embodiments of the invention. These embodiments are given for the purpose of disclosure.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference herein.
Alfa, C., Fantes, P., Hyams, J., McLeod, M., and Warbrick, E. (1993). Nutritional control of entry into stationary phase determined by flow fluorocytometry. In Experiments with Fission Yeast, A Laboratory Course Manual (Cold Spring Harbor, N.Y., Cold Spring Harbor Laboratory), pp. 186.
al-Khodairy, F., and Carr, A. M. (1992). DNA repair mutants defining G2 checkpoint pathways in Schizosaccharomyces pombe. Embo J 11, 1343-1350. Boddy, M., Furnari, B., Mondesert, O., and Russell, P. (1998). Replication checkpoint enforced by kinases Cds1 and Chk1. Science 280, 909-912.
Chay, C. A., Gunasinge, U. B., Dinesh-Kumar, S. P., Miller, W. A., and Gray, S. M. (1996). Aphid transmission and systemic plant infection determinants of barley yellow dwarf luteovirus-PAV are contained in the coat protein readthrough domain and 17-kDa protein, respectively. Virology 219, 57-65.
Chen, M., Yang, J., Wang, B., Elder, R. T., Song, X.-q., Saksena, N., and Zhao, Y. (2000). Functional Conservation of HIV-1 Viral Protein R (Vpr) and Its Association with Disease Progression of HIV-infected Patients. 8th Conference on Retroviruses and Opportunistic Infections, Chicago, Accepted.
Edwards M C. Mapping of the seed transmission determinants of barley stripe mosaic virus. Mol Plant Microbe Interact. 1995, 8: 906-915.
Elder, R. T., Benko, Z., and Zhao, Y. (2002). HIV-1 VPR modulates cell cycle G2/M transition through an alternative cellular mechanism other than the classic mitotic checkpoints. Front Biosci 7, d349-357.
Elder, R. T., Yu, M., Chen, M., Edelson, S., and Zhao, Y. (2000). Cell cycle G2 arrest induced by HIV-1 Vpr in fission yeast (Schizosaccharomyces pombe) is independent of cell death and early genes in the DNA damage checkpoint. Viral Research 68, 161-173.
Elder, R. T., Yu, M., Chen, M., Zhu, X., Yanagida, M., and Zhao, Y. (2001). HIV-1 Vpr induces cell cycle G2 arrest in fission yeast (Schizosaccharomyces pombe) through a pathway involving regulatory and catalytic subunits of PP2A and acting on both Wee1 and Cdc25. Virology 287, 359-370.
Enoch, T., and Nurse, P. (1990). Mutation of fission yeast cell cycle control genes abolishes dependence of mitosis on DNA replication. Cell 60, 665-673.
Fransz, P., Armstrong, S., Alonso-Blanco, C., Fischer, T. C., Torres-Ruiz, R. A., and Jones, G. (1998). Cytogenetics for the model system Arabidopsis thaliana. Plant J 13, 867-876.
Funabiki, H., Kumada, K., and Yanagida, M. (1996). Fission yeast Cut1 and Cut2 are essential for sister chromatid separation, concentrate along the metaphase spindle and form large complexes. Embo J 15, 6617-6628.
Furnari, B., Blasina, A., Boddy, M. N., McGowan, C. H., and Russell, P. (1999). Cdc25 inhibited in vivo and in vitro by checkpoint kinases Cds1 and Chk1. Mol Biol Cell 10, 833-845.
Furnari, B., Rhind, N., and Russell, P. (1997). Cdc25 mitotic inducer targeted by Chk1 DNA damage checkpoint kinase. Science 277, 1495-1497.
Goshima, G., Iwasaki, O., Obuse, C., and Yanagida, M. (2003). The role of Ppe1/PP6 phosphatase for equal chromosome segregation in fission yeast kinetochore. Embo J 22, 2752-2763.
Gould, K. L, Moreno, S., Tonks, N. K., and Nurse, P. (1990). Complementation of the mitotic activator, p80cdc25, by a human protein-tyrosine phosphatase. Science 250, 1573-1.576.
Gould, K. L., and Nurse, P. (1989). Tyrosine phosphorylation of the fission yeast cdc2+protein kinase regulates entry into mitosis. Nature 342, 39-45.
Hayles, J., and Nurse, P. (1995). A pre-start checkpoint preventing mitosis in fission yeast acts independently of p34cdc2 tyrosine phosphorylation. EMBO J. 14, 2760-2771.
Kinoshita, N., Ohkura, H., and Yanagida, M. (1990). Distinct, essential roles of type 1 and 2A protein phosphatases in the control of the fission yeast cell division cycle. Cell 63, 405-415.
Kornitzer, D., Sharf, R., and Kleinberger, T. (2001). Adenovirus E4orf4 protein induces PP2A-dependent growth arrest in Saccharomyces cerevisiae and interacts with the anaphase-promoting complex/cyclosome. J Cell Biol 154, 331-344.
Kovelman, R., and Russell, P. (1996). Stockpiling of Cdc25 during a DNA replication checkpoint arrest in Schizosaccharomyces pombe. Mol Cell Biol 16, 86-93.
Krek, W., and Nigg, E. A. (1991). Differential phosphorylation of vertebrate p34cdc2 kinase at the G1/S and G2/M transitions of the cell cycle: identification of major phosphorylation sites. EMBO Journal 10, 305-316.
Lee, M., and Nurse, P. (1988). Cell cycle control genes in fission yeast and mammalian cells. Trends Genet. 4, 287-290.
Lee, M. G., and Nurse, P. (1987). Complementation used to clone a human homologue of the fission yeast cell cycle control gene cdc2. Nature 327, 31-35.
Lundgren, K., Walworth, N., Booher, R., Dembski, M., Kirschner, M., and Beach, D. (1991). mik1 and wee1 cooperate in the inhibitory tyrosine phosphorylation of cdc2. Cell 64, 1111-1122.
MacKintosh, R. W., Haycox, G., Hardie, D. G., and Cohen, P. T. (1990). Identification by molecular cloning of two cDNA sequences from the plant Brassica napus which are very similar to mammalian protein phosphatases-1 and -2A. FEBS Lett 276, 156-160.
MacNeill, S. A., Creanor, J., and Nurse, P. (1991). Isolation, characterisation and molecular cloning of new mutant alleles of the fission yeast p34cdc2+protein kinase gene: identification of temperature-sensitive G2-arresting alleles. Mol Gen Genet. 229, 109-118.
Masuda, M., Nagai, Y., Oshima, N., Tanaka, K., Murakami, H., Igarashi, H., and Okayama, H. (2000). Genetic studies with the fission yeast Schizosaccharomyces pombe suggest involvement of wee1, ppa2, and rad24 in induction of cell cycle arrest by human immunodeficiency virus type 1 Vpr. J Virol 74, 2636-2646.
Maundrell, K. (1993). Thiamine-repressible expression vectors pREP and pRIP for fission yeast. Gene 123, 127-130.
Mayo, M. A., and Ziegler-Graff, V. (1996). Molecular biology of luteoviruses. Adv Virus Res 46, 413-460.
Meszaros, T., Miskolczi, P., Ayaydin, F., Pettko-Szandtner, A., Peres, A., Magyar, Z., Horvath, G. V., Bako, L., Feher, A., Dudits, D., et al. (2000). Multiple cyclin-dependent kinase complexes and phosphatases control G2/M progression in alfalfa cells Conserved and novel regulators of the plant cell cycle. Plant Mol Biol 43, 595-605.
Miller, W. A., and Rasochova, L. (1997). Barley yellow dwarf viruses. Annu Rev Phytopathol 35, 167-190.
Ministry of Agriculture and Food, C. (2003). Cereals: Barley Yellow Dwarf Virus. In Agronomy Guide For Field Crops, O. Staff, ed. (Ontario, Ministry of Agriculture and Food, Canada).
Minshull, J., Straight, A., Rudner, A. D., Dernburg, A. F., Belmont, A., and Murray, A. W. (1996). Protein phosphatase 2A regulates MPF activity and sister chromatid cohesion in budding yeast. Curr Biol 6, 1609-1620.
Morgan, D. O. (1995). Principles of CDK regulation. Nature 374, 131-134.
Murata, M., Heslop-Harrison, J. S., Motoyoshi, F., Fransz, P. F., Alonso-Blanco, C., Liharska. T. B., Peeters, A. J., Zabel, P., and de Jong, J. H. (1997). Physical mapping of the 5S ribosomal. RNA genes in Arabidopsis thaliana by multi-color fluorescence in situ hybridization with cosmid clones. Plant J 12, 31-37.
Nass, P. H., Domier, L. L., Jakstys, B. P., and D'Arcy, C. J. (1998). In situ localization of barley yellow dwarf virus-PAV 17-kDa protein and nucleic acids in oats. Phytopathology 88, 1031-1039.
Norbury, C., Blow, J., and Nurse, P. (1991). Regulatory phosphorylation of the p34cdc2 protein kinase in vertebrates. EMBO J. 10, 3321-3329.
Nurse, P. (1997). Checkpoint pathways come of age. Cell 91, 865-867.
Nurse, P., Thuriaux, P., and Nasmyth, K. (1976). Genetic control of the cell division cycle in the fission yeast Schizosaccharomyces pombe. Mol Gen Genet. 146, 167-178.
Parker, L. L., Walter, S. A., Young, P. G., and Piwnica-Worms, H. (1993). Phosphorylation and inactivation of the mitotic inhibitor Wee1 by the nim1/cdr1 kinase. Nature 363, 736-738.
Rhind, N., and Russell, P. (1998). Mitotic DNA damage and replication checkpoints in yeast. Curr Opin Cell Biol 10, 749-758.
Schmitz, J., Stussi-Garaud, C., Tacke, E., Prufer, D., Rohde, W., and Rohfritsch, O. (1997). In situ localization of the putative movement protein (pr17) from potato leafroll luteovirus (PLRV) in infected and transgenic potato plants. Virology 235, 311-322.
Smith, H. G., and Barker, H. (1999). The Luteoviridae, Oxford University Press).
Tacke, E., Prufer, D., Schmitz, J., and Rohde, W. (1991). Thepotato leafroll luteovirus 17K protein is a single-stranded nucleic acid-binding protein. J Gen Virol 72 (Pt 8), 2035-2038.
Wang, Y., and Burke, D. J. (1997). Cdc55p, the B-type regulatory subunit of protein phosphatase 2A, has multiple functions in mitosis and is required for the kinetochore/spindle checkpoint in Saccharomyces cerevisiae. Mol Cell Biol 17, 620-626.
Zeng, Y., Forbes, K. C., Wu, Z., Moreno, S., Piwnica-Worms, H., and Enoch, T. (1998). Replication checkpoint requires phosphorylation of the phosphatase Cdc25 by Cds1 or Chk1. Nature, 395, 507-510.
Zhao, Y., Cao, J., O'Gonnan, M. R. G., Yu, M., and Yogev, R. (1996). Effect of human immunodeficiency virus Type 1 protein R (vpr) gene expression on basic cellular functions of fission yeast Schizosaccharomyces pombe. J Virol 70, 5821-5826.
Zhao, Y., Elder, R. T., Chen, M., and Cao, J. (1998a). Fission yeast expression vectors adapted for large scale cloning and GFP fusion with positive screening. BioTechniques 25, 438-444.
   Zhao, Y., and Lieberman, H. B. (1995). Schizosaccharomyces pombe: a model for molecular studies of eukaryotic genes. DNA Cell Biol 14, 359-371.
Zhao, Y., Yu, M., Chen, M., Elder, R. T., Yamamoto, A., and Cao, J. (1998b). Pleiotropic Effects of HIV-1 Protein R (Vpr) on Morphogenesis and Cell Survival in Fission Yeast and Artagonism by Pentoxifylline. Virology 246, 266-276.
Ziegler-Graff, V., Brault, V., Mutterer, J. D., Simonis, M., Herrbach, E., Guilley, H., Richards, K. E., and Jonard, G. (1996). The coat protein of beet yellows luteovirus is essential for systemic infection but the viral gene products of P29 and P19 are dispensable for systemic infection and aphid transmission. Mol Plant-Microbe Interact 6, 501-510.
Zuo, J., Niu, Q. W., and Chua, N. H. (2000). Technical advance: An estrogen receptorbased transactivator XVE mediates highly inducible gene expression in transgenic plants. Plant J 24, 265-273.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A transgenic plant resistant to barley yellow dwarf virus (BYDV) infection, wherein the plant comprises a plurality of plant cells transformed with a vector that expresses inhibitory RNA that downregulates expression, transcription or translation of BYDV MP.

2. The transgenic plant of claim 1, wherein the inhibitory RNA is anti-sense RNA.

3. The transgenic plant of claim 1, wherein the inhibitory RNA is cosuppressor RNA.

4. The transgenic plant according to claim 1, wherein the plant is selected from the group comprising A. thaliana, tobacco, barley, wheat, oats, and corn.

5. The transgenic plant according to claim 1, wherein the vector is a viral vector obtained from a positive single-stranded RNA plant virus.

6. The transgenic plant according to claim 5, wherein the positive single-stranded RNA plant virus is a tobamovirus.

7. The transgenic plant according to claim 6, wherein the tobamovirus is a tobacco mosaic virus.

8. A method of identifying a suppressor of barley yellow dwarf virus movement protein (BYDV MP), comprising:
(a) providing a candidate suppressor;
(b) admixing the candidate suppressor with an isolated compound, cell, or suitable experimental animal to produce a recombinant compound, cell, or suitable experimental animal;
(c) measuring one or more characteristics of the recombinant compound, cell, or animal in step (b); and
(d) comparing the characteristic measured in step (c) with the characteristic of the compound, cell, or animal in the absence of said candidate suppressor,
wherein a difference between the measured characteristics indicates that said candidate suppressor is a suppressor of the compound, cell, or animal.

9. The method of claim 8, wherein the candidate suppressor is a nucleic acid, protein, or small molecule.

10. The method of claim 8, wherein the nucleic acid is inhibitory RNA.

11. The method of claim 8, wherein the cell is an eukaryotic cell.

12. The method of claim 8, wherein the cell is a prokaryotic cell.

13. The method of claim 8, wherein the animal is a mouse, Xenopus, zebrafish, rat, Drosophila, or C. elegans.

14. A method of screening for a suppressor that inhibits barley yellow dwarf virus (BYDV) movement protein (MP) activity, comprising:
(a) contacting one or more cells with a test agent, wherein the one or more cells comprise a BYDV MP gene or a variant thereof under control of a promoter;
(b) growing the culture under conditions suitable to induce expression of the BYDV MP gene or variant thereof; and
(c) screening the one or more cells in (a) for a cell characteristic and/or phenotype not present in a control cell or cell culture, wherein the presence of the cell characteristic or cell phenotype indicates that the test agent is a suppressor that downregulates BYDV MP activity.

15. The method of claim 14, wherein the BYDV MP gene is integrated into the genome of the cell.

16. The method of claim 14, wherein the promoter is an inducible promoter.

17. The method of claim 14, wherein the one or more cells are yeast cells.

18. The method of claim 14, wherein the cell characteristic comprises increased viability compared to the control culture, cytotoxicity; chromosomal abnormality; or cellular growth.

19. The method of claim 14, wherein the cell phenotype is normal cell length and size, change of cell morphology; orchromosomal abnormality.

20. The method of claim 14, wherein the test agent prevents BYDV MP-induced cell cycle G2 arrest.

21. The method of claim 14, wherein the yeast is a fission yeast or a budding yeast.

22. The method of claim 21, wherein the fission yeast is Schizosaccharomyces pombe.

23. The method of claim 21, wherein the budding yeast is Saccharomyces cerevisiae.

24. (canceled)

25. (canceled)

26. (canceled)

27. A viricide composition comprising the BYDV MP suppressor of claim 8.

28. An isolated eukaryotic cell transformed with a vector, said vector comprising DNA encoding barley yellow dwarf virus (BYDV) movement protein (MP) or a variant thereof, wherein said DNA is operably linked to one or more regulatory elements for expression of the DNA in the cell.

29. The isolated cell of claim 28, wherein said cell is a yeast cell.

30. An in vitro screening method to identify an agent that binds to BYDV MP or a variant thereof, comprising:
(a) subjecting BYDV MP or variant thereof to a test agent under conditions that allow the BYDV MP or variant thereof to bind the test agent; and
(b) detecting the binding of the BYDV MP or variant thereof with the test agent, wherein the detection of the binding identifies the test agent as an agent that binds to BYDV MP or to the variant.

31. The method of claim 30, wherein the screening occurs in a multi-well plate, single agar plate, liquid growth medium, cell, tissue, or organism.

32. (canceled)

33. (canceled)

34. (canceled)

35. (canceled)

36. (canceled)

37. (canceled)

38. (canceled)

39. (canceled)

40. (canceled)

41. (canceled)


SEQUENCE LISTING (2 sequences)

SEQ ID NO: 1 - protein, 153 aa, Barley yellow dwarf virus
MAQGEQGALAQFGEWLWSNPIEPDQNEELVDAQEEEGQILYLDQQAGMRYSYSQSTTLRPTPQGQSSVPTFRNAQRFQVEYSSPTTVTRSQTSRLSLSHTRPPLQSAQCLLNSTLRAHNQPWVATLTHSPSQNQQPKTSPPNRLTGRNSGRAR

SEQ ID NO: 2 - DNA, 881 bp, Barley yellow dwarf virus
atggcccaaggagagcaaggcgcgctggcgcagttcggagaatggttgtggtccaacccaatcgagccggaccaaaacgaagaactcgtcaccaacaccaggttgggttagctcggcctggttttgcttcttgagcaggacgcacaagaggaggaggggcaaatcttatatctggaccagcaggcaggcctgcgtgttctcctcctccccgtttagaatatagacctggtcgtccgtccgatgaggtattcgtattctcagtcaacgaccttaaggccaactcctcagggatactccataagcataagagtcagttgctggaattccggttgaggagtccctcaatcaagttcggtcccgacctttcgcaatgcccagcgctttcaggtggaatactcagttagttcaagccagggctggaaagcgttacgggtcgcgaaagtccaccttatgagtagtcctaccaccgttacaagatcacaaacgtcaaggttgagtttaagtcactcaggatggtggcaatgttctagtgtttgcagttccaactcaaattcagtgacgcgtccgcctctacagtcggcgcaatgtttattgaactcgacacttcgttgcgcaggcggagatgtcagccgcgttacaaataacttgagctgtgaagcagctcacaatcaaccttgggtagctacattaactcattcaccctctcaacgagtgttagttggaacccatcgatgtaattgagtaagtgggagagttcagcaaccaaaaacttcaccgcccaacagattgacgggaaggaattcagtcgttggtttttgaagtggcgggttgtctaactgcccttccttaaggggagagctcggtgaccctctcgagccact